(19) United States
(12) Patent Application Publication
(10) Pub. No.: US 2025/0017864 A1
(43) Pub. Date: Jan. 16, 2025

(54) COMPOSITIONS FOR CELL-BASED THERAPIES AND RELATED METHODS

(71) Applicant: SIGILON THERAPEUTICS, INC., Cambridge, MA (US)

(72) Inventors: Lauren Emily Barney, Cambridge, MA (US); Hozefa Bandukwala, Southboroough, MA (US); Susan J. Drapeau, Arlington, MA (US); Lauren E. Jansen, Roslindale, MA (US)

(21) Appl. No.: 18/705,759

(22) PCT Filed: Oct. 28, 2022
(86) PCT No.: PCT/US2022/048256

§ 371 Date: Apr. 29, 2024

Related U.S. Application Data
(60) Provisional application No. 63/273,638, filed on Oct. 29, 2021.

(51) Int. Cl. A61K 9/50; A61K 35/30; A61K 45/06; A61K 47/02; A61K 47/18; A61K 47/26

(52) U.S. Cl. CPC A61K 9/5036; A61K 35/30; A61K 45/06; A61K 47/02; A61K 47/183; A61K 47/26

(57) ABSTRACT

Described herein are hydrogel capsule compositions which comprise a population of hydrogel capsules and a pharmaceutically acceptable solution. The hydrogel capsules in the population comprise a hydrogel-forming polymer and encapsulate a plurality of cells.

### CLAIM OF PRIORITY

This application claims priority to U.S. Provisional Application No. 63/273,678, filed Oct. 29, 2021. The entire disclosure of the foregoing application is incorporated by reference in its entirety.

### BACKGROUND

Treating chronic and genetic diseases by implanting living cells that produce a therapeutic substance capable of treating such diseases has exciting potential to improve the health of patients with such diseases. To fully achieve this potential, the implanted cells must be protected from the patient's immune response, so that they remain viable, and the implanted cells must also be capable of producing therapeutic levels of the desired therapeutic substance for several weeks, months or even longer. One exploratory approach for delivering such cell-based therapies is to encapsulate the therapeutic-producing cells in alginate hydrogel capsules, with the objective that the capsule structure isolates the therapeutic-producing cells from immune system cells, while allowing entry of nutrients for the therapeutic-producing cells and exit of the therapeutic substance from the capsules. Once such hydrogel capsules are produced, they need to be stored in a manner that preserves the integrity of the capsule structure and viability of the encapsulated cells.

### SUMMARY

In one aspect, the present disclosure provides a hydrogel capsule composition, which comprises a population of hydrogel capsules and a pharmaceutically acceptable solution, wherein each hydrogel capsule in the population comprises an ionically cross-linked alginate and encapsulates a plurality of live mammalian cells. In an embodiment, the solution comprises a calcium salt at an elemental calcium concentration of between about 1.0 mM and about 10 mM. In an embodiment, the elemental calcium concentration is between about 1.2 mM and about 3 mM. In an embodiment, the calcium salt is calcium chloride. In an embodiment, the solution has an osmolality of about 250 mOsm/kg to about 350 mOsm/kg and a pH of between 6.0 and 9.0 at a temperature of 12° C. to 30° C.

In some embodiments, the solution further comprises at least one buffering agent capable of maintaining the pH of the solution within a desired range (e.g., between 6.0 and 9.0) when the composition is stored at a temperature of 12° C. to 30° C. (e.g., about 15-25° C.). In an embodiment, the buffering agent comprises one or more of an acetate salt (e.g., sodium acetate), a gluconate salt (e.g., sodium gluconate), a bicarbonate salt (e.g., sodium bicarbonate), a lactate salt (e.g., sodium lactate), or 4-(2-hydroxyethyl)-1-piperazineethanesulfonic acid (HEPES).

In an embodiment, the solution further comprises a carbon source (e.g., a sugar (e.g., dextrose, glucose, galactose, hexose, fructose, maltose), glycerol, glutamine, pyruvate) that supports viability of the encapsulated cells. In an embodiment, the carbon source is glucose. In an embodiment, the solution comprises about 1.5 mM to 2.5 mM calcium chloride, about 5 mM to about 25 mM D-glucose, and about 40 mM to about 50 mM sodium bicarbonate.

In some embodiments, the solution further comprises minerals, amino acids and vitamins that support viability of the encapsulated cells.

In an embodiment, the minerals include a magnesium salt (e.g., magnesium chloride or magnesium sulfate) or a potassium salt (e.g., potassium chloride).

In an embodiment, the amino acids include histidine, isoleucine, leucine, lysine, methionine, phenylalanine, threonine, tryptophan, and valine.

In an embodiment, the vitamins include a vitamin B1 compound (e.g., thiamine or a thiamine salt, e.g., thiamine hydrochloride), a vitamin B3 compound (e.g., nicotinic acid or niacinamide) and a vitamin B6 compound (e.g., pyroxidine or a pyroxidine salt, e.g., pyroxidine hydrochloride).

In some embodiments, the amino acids include L-arginine, L-cystine, L-glycine, L-histidine, L-isoleucine, L-leucine, L-lysine, L-methionine, L-phenylalanine, L-serine, L-threonine, L-tryptophan, L-tyrosine and L-valine.

In some embodiments, the vitamins include choline or a choline salt (e.g., choline chloride), a vitamin B1 compound (e.g., thiamine or a thiamine salt, e.g., thiamine hydrochloride), a vitamin B3 compound (e.g., nicotinic acid or niacinamide), a vitamin B5 compound (e.g., pantotheneic acid or calcium pantothenate), a vitamin B6 compound (e.g., pyroxidine or a pyroxidine salt, e.g., pyroxidine hydrochloride), a folate compound (e.g., folic acid), riboflavin and i-inositol.

In an embodiment, the hydrogel capsules in the hydrogel capsule population are sphere-like or spherical in shape. In an embodiment, the hydrogel capsules have a mean capsule diameter of about 500 μm to about 5000 μm (e.g., about 500 μm to about 4000 μm, about 500 μm to about 3000 μm, about 500 μm to about 2500 μm, about 500 μm to about 2000 μm, about 500 μm to about 1500 μm, about 500 μm to about 1000 μm, about 1000 μm to about 2500 μm). In an embodiment, the hydrogel capsules are not sphere-like or spherical in shape. In an embodiment, each capsule comprises a cell-containing compartment surrounded by a barrier compartment which comprises the ionically cross-linked alginate. In an embodiment, the cross-linked alginate comprises an alginate covalently modified with at least one afibrotic compound, as defined herein. In an embodiment, the cross-linking agent comprises barium ions. In some embodiments, the cell containing compartment encapsulates the live mammalian cells in a first polymer composition comprising an alginate, which is optionally ionically cross-linked (e.g., with barium ions). In some embodiments, the alginate in the first polymer composition is covalently modified with a cell-contacting peptide, as defined herein. In an embodiment, the mean capsule diameter of the hydrogel capsules is 1400 to 2000 μm.

In some embodiment, the encapsulated live mammalian cells are derived from a human cell, e.g., from an RPE cell (e.g., an ARPE-19 cell). In an embodiment, the cells are genetically modified to express and secrete an exogenous protein, e.g., any of the therapeutic proteins described herein.

In another aspect, the present disclosure provides a sealed container comprising any of the capsule compositions described herein. Each interior surface of the container in contact with the composition consists essentially of a biocompatible material, e.g., a medical grade plastic (e.g., fluorinated ethylene propylene (FEP) or polyethylene terephthalate glycol (PETG)). In an embodiment, the bottom interior surface of the container has a rectangular or round shape.

In an embodiment, substantially all (e.g., at least 90%, 95%, 98% or greater) of the hydrogel capsules in the composition are distributed substantially uniformly across the bottom interior surface of the container in a capsule layer and a volume of the solution (VS) is disposed on top of the layer. In an embodiment, the capsule layer has a depth equal to about 1.00 to about 1.25 times the average diameter of the capsules in the composition.

In yet another aspect, the present disclosure features a method of making a sealed container comprising a hydrogel capsule composition described herein. In an embodiment, the method comprises providing a population of hydrogel capsules encapsulating live mammalian cells, combining the population of capsules with a pharmaceutically acceptable aqueous solution described herein and placing a desired volume of the composition into a biocompatible, sealable container in a manner that produces a capsule layer in which substantially all of the capsules in the composition volume are distributed substantially uniformly across the bottom of the container at a depth equal to about 1.00 to about 1.25 times the mean diameter of the capsules in the composition.

In an embodiment, the method further comprises adding a desired volume of the pharmaceutically acceptable solution in a manner to form a solution layer on top of the capsule layer, and sealing the container. In an embodiment, the method further comprises storing the sealed container for a desired time period at a temperature of 2° C. to 30° C. or about 12° C. to 30° C. (e.g., about 15-25° C.). for a desired time period and assessing the viability of the encapsulated cells in the composition at one or more time points during the desired time period.

In a still further aspect, the present disclosure features the use of a hydrogel capsule composition described herein for treating a mammalian subject (e.g., a human subject) in need of treatment with a substance (e.g., a protein) produced by the encapsulated cells in the composition.

In an embodiment, the subject is treated by a method comprising administering a desired amount of the composition to the subject, e.g., by implanting the desired amount into the intraperitoneal cavity of the subject. In an embodiment, the method comprises providing the hydrogel capsule composition in a sealed container described herein, unsealing the container, and removing the desired quantity. In an embodiment, the encapsulated cells in the hydrogel capsule composition are genetically modified to produce one of the exogenous proteins described herein.

### BRIEF DESCRIPTION OF THE DRAWINGS

FIG. 1 shows a graph of viability of cells encapsulated in alginate hydrogel spheres and stored for up to three days in an aqueous solution that lacked calcium or in the same solution with added calcium, with the error bars representing the standard deviation (SD) between two replicates per time point.

FIG. 2 shows a graph of plasma FVIII levels in mice implanted with alginate hydrogel spheres encapsulating FVIII-expressing cells after the spheres had been stored in different storage solutions, with the error bars representing the standard deviation (SD) between four mice per storage condition.

FIGS. 3A and 3B show graphs of plasma FVIII levels in mice implanted with alginate hydrogel spheres encapsulating FVIII-expressing cells after the spheres had been stored in different storage solutions for the indicated time period, with the error bars representing the standard deviation (SD) between four mice per storage condition.

FIG. 4 shows FVIII levels secreted into conditioned media from alginate hydrogel spheres encapsulating FVIII-expressing cells after the sphere had been stored for up to seven days as a monolayer, bilayer, or trilayer, with the average of two duplicate sample graphed as a single point.

FIG. 5 shows a graph of plasma FVIII levels in mice implanted with alginate hydrogel spheres encapsulating FVIII-expressing cells after the spheres had been stored in different storage solutions, with the error bars representing the standard deviation (SD) between four mice per storage condition.

### DETAILED DESCRIPTION

The present disclosure features a hydrogel capsule composition comprising a population of live mammalian cells (e.g., human RPE cells) in a pharmaceutically acceptable solution, a sealed container comprising the hydrogel capsule composition, and =uses thereof for treating a mammalian subject in need of treatment with a substance produced by the encapsulated cells. Various embodiments will be described below.

### Abbreviations and Definitions

Throughout the detailed description and examples of the disclosure the following abbreviations will be used.

| | |
|---|---|
| CM-Alg | chemically modified alginate |
| CM-LMW-Alg | chemically modified, low molecular |
| | weight alginate |
| CM-LMW-Alg-101 | low molecular weight alginate, |
| | chemically modified with Compound |
| | 101 shown in Table 4 |
| CM-HMW-Alg | chemically modified, high molecular |
| | weight alginate |
| CM-HMW-Alg-101 | high molecular weight alginate, |
| | chemically modified with Compound |
| | 101 shown in Table 4 |
| CM-MMW-Alg | chemically modified, medium |
| | molecular weight alginate |
| CM-MMW-Alg-101 | medium molecular weight alginate, |
| | chemically modified with |
| | Compound 101 shown in Table 4 |
| HMW-Alg | high molecular weight alginate |
| MMW-Alg | medium molecular weight alginate |
| U-Alg | unmodified alginate |
| U-HMW-Alg | unmodified high molecular weight alginate |
| U-LMW-Alg | unmodified low molecular weight alginate |
| U-MMW-Alg | unmodified medium molecular weight alginate |
| 70:30 CM-Alg:U-Alg | 70:30 mixture (V:V) of a chemically |
| | modified alginate and an unmodified alginate, |
| | e.g., as described in WO2020069429 |

### Definitions

So that the disclosure may be more readily understood, certain technical and scientific terms used herein are specifically defined below. Unless specifically defined elsewhere in this document, all other technical and scientific terms used herein have the meaning commonly understood by one of ordinary skill in the art to which this disclosure belongs.

As used herein, including the appended claims, the singular forms of words such as “a,” “an,” and “the,” include their corresponding plural references unless the context clearly dictates otherwise.

“About” or “approximately” means when used herein to modify a numerically defined parameter (e.g., a physical description of a hydrogel capsule such as diameter, sphericity, number of cells encapsulated therein, the number of capsules in a preparation), means that the recited numerical value is within an acceptable functional range for the defined parameter as determined by one of ordinary skill in the art, which will depend in part on how the numerical value is measured or determined, e.g., the limitations of the measurement system, including the acceptable error range for that measurement system. For example, “about” can mean a range of 20% above and below the recited numerical value. As a non-limiting example, a hydrogel capsule defined as having a diameter of about 1.5 millimeters (mm) and encapsulating about 5 million (M) cells may have a diameter of 1.2 to 1.8 mm and may encapsulate 4 M to 6 M cells. As another non-limiting example, a preparation of about 100 hydrogel capsules includes preparations having 80 to 120 capsules. In some embodiments, the term “about” means that the modified parameter may vary by as much as 15%, 10% or 5% above and below the stated numerical value for that parameter.

“Acquire” or “acquiring”, as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., using a fluorescence microscope to acquire fluorescence microscopy data.

“Administer”, “administering”, or “administration”, as used herein, refer to implanting, absorbing, ingesting, injecting, or otherwise introducing into a subject, an entity described herein (e.g., a hydrogel capsule composition or aliquot thereof), or providing such an entity to a subject for administration.

“Afibrotic”, as used herein in reference to a compound, polymer or other substance, means that the substance mitigates the foreign body response (FBR). For example, the amount of FBR in a biological tissue that is induced by implant into that tissue of a hydrogel capsule comprising an afibrotic compound (e.g., a polymer covalently modified with a compound listed in Table 4) is lower than the FBR induced by implantation of an afibrotic-null reference capsule that lacks any afibrotic compound, but is of substantially the same composition (e.g., same cell type(s)) and structure (e.g., size, shape, no. of compartments). In an embodiment, the degree of the FBR is assessed by the immunological response in the tissue containing the implanted hydrogel capsule, which may include, for example, protein adsorption, macrophages, multinucleated foreign body giant cells, fibroblasts, and angiogenesis, using assays known in the art, e.g., as described in WO 2017/075630, WO 2021/119522 or using one or more of the assays/methods described Vegas, A., et al., Nature Biotechnol (supra). In an embodiment, an afibrotic compound is a compound of Formula (I):

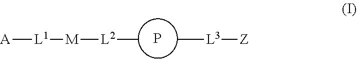

or a pharmaceutically acceptable salt thereof, wherein the variables A, L¹, M, L², P, L³, and Z, as well as related subvariables, are defined herein.

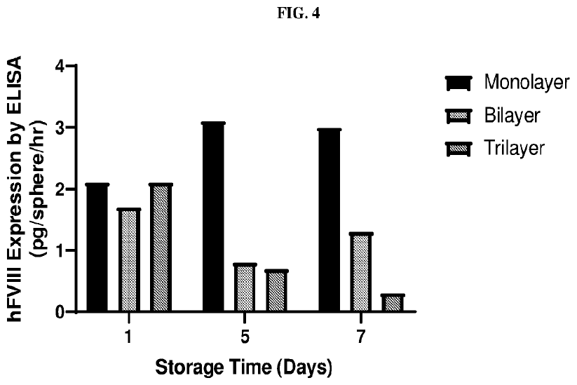

“Alpha-galactosidase A”, “α-Gal A”, “alpha-D-galactosidase-A”, alpha-galactoside galactohydrolase”, “galactosidase alpha”, and “GLA protein” may be used interchangeably herein and refer to a protein comprising the mature amino acid sequence of a mature, wild-type mammalian ARSB or any fragment, mutant, variant or derivative thereof that has enzyme activity that is within 80-120%, 85-115%, 90-110% or 95-105% of the corresponding wild-type mammalian mature GLA protein, as measured by any art-recognized GLA activity assay. GLA hydrolyzes the terminal alpha-D-galactose residues in glycosphingolipids, particularly in globotriaosylceramide (Gb₃). The wild-type human GLA gene encodes a 429-amino acid polypeptide, of which the N-terminal 31 amino acids constitute a signal peptide (GenBank Accession No. CAA29232.1). In an embodiment, the GBA protein is part of a fusion protein that further comprises one or more amino acids sequences from one or more heterologous polypeptides. In an embodiment, the encapsulated cells comprise an exogenous nucleotide sequence that encodes the GLA fusion protein shown in FIG. 4A of WO 2020/198685.

GLA activity can be directly measured by obtaining blood leukocytes from a subject, lysing of the leukocytes, and determining the enzymatic activity in the lysate upon addition of an enzyme substrate such as 4-methylumbelliferal alpha-D-galactoside. Immunoassays for measuring GLA activity and protein to determine the concentrations of alpha-galactosidase in blood and plasma are described in Fuller et al., Clin Chem. 2004; 50(11):1979-85. Indirect assessments of GLA activity are based on measuring a substrate e.g., levels of Gb3 and biomarker lysoGb3 in blood plasma and/or urine sample collected from the subject or in a biopsy of a tissue of interest, e.g., liver, kidney, heart. Gb3 and lysoGb3 levels can be measured using any art-recognized assay. For example, a method for measuring Gb3 levels in plasma and urine of humans affected by Fabry disease is described in, e.g., Boscaro et al., Rapid Commun Mass Spectrom. 2002; 16(16):1507-14. Gb3 accumulation in skin biopsies obtained using a “punch” device may be detected using an immunoelectron-microscopic method such as described in Kanekura et al., Br J Dermatol. 2005, 153(3):544-8. Various biopsy techniques and assays for detecting Gb3 and other surrogate biomarkers are described in US patent application publication US 2010/0113517. Other plasma surrogate biomarkers of GLA activity and/or Fabry disease progression (e.g., various inflammatory and cardiac remodeling biomarkers) are described in Yogasundaram, H. et al., J Am Heart Assoc. 2018; 7:e009098.

“Alpha-L-iduronidase protein” and “IDUA protein” may be used interchangeably herein and refer to a protein that (i) is capable of hydrolyzing nonreducing terminal alpha-L-iduronic acid residues in glycosaminoglycans (GAGs) (e.g., dermatan sulfate and heparan sulfate) and (ii) comprises the mature amino acid sequence of a mature, wild-type mammalian IDUA protein or any fragment, mutant, variant or derivative thereof that has enzymatic activity that is within 80-120%, 85-115%, 90-110% or 95-105% of the corresponding wild-type mammalian mature IDUA protein, as measured by any art-recognized IDUA activity assay (e.g., hydrolysis of the substrate 4-methylumbelliferyl-α-L-iduronide (4MU-iduronide), see, e.g., Ou, L. et al., Mol Genet Metab. 2014 February: 111(2): 113-115). The wild-type human IDUA gene encodes a 653 amino acid precursor protein, of which the N-terminal 26 amino acids constitute a signal peptide (GenBank Accession No. AAA81589.1, GenBank Accession No. AAA51698.1). In an embodiment, the IDUA protein is part of a fusion protein that further comprises one or more amino acids sequences from one or more heterologous polypeptides. “Arylsulfatase B protein” and “ARSB protein” may be used interchangeably herein and refer to a protein (i) that is capable of hydrolyzing the 4-sulfate groups of the N-acetyl-D-galactosamine 4-sulfate units of chondroitin sulfate and dermatan sulfate and (ii) comprises the amino acid sequence of a mature, wild-type mammalian ARSB or any fragment, mutant, variant or derivative thereof that has enzymatic activity that is within 80-120%, 85-115%, 90-110% or 95-105% of the corresponding wild-type mammalian mature ARSB protein, as measured by any ARSB activity assay known in the art. The wild-type human ARSB gene encodes a 533 amino acid precursor polypeptide, of which the N-terminal 36 or 38 amino acids constitute a signal peptide (UniProtKB—P15848). In an embodiment, the ARSB protein is part of a fusion protein that further comprises one or more amino acids sequences from one or more heterologous polypeptides.

“Beta-glucosidase protein”, “acid beta-glucocerebrosidase protein”, glucosylceramidase beta protein” and “GBA protein”, may be used interchangeably herein to refer to a protein that comprises the mature amino acid sequence encoded by a wild-type mammalian GBA gene or any fragment, mutant, variant or derivative thereof that has enzyme activity that is within 80-120%, 85-115%, 90-110% or 95-105% of the corresponding wild-type mammalian mature GBA protein, as measured by any GBA assay known in the art. GBA catalyzes the breakdown of the glycolipid glucosylceramide (GlcCer) to ceramide and glucose. The wild-type human GBA gene encodes a 536 amino acid precursor polypeptide (UniProtKB—P04062-1). In an embodiment, the GBA protein is part of a fusion protein that further comprises one or more amino acids sequences from one or more heterologous polypeptides.

“Cell,” as used herein, refers to a genetically modified cell or a cell that is not genetically modified. In an embodiment, a cell is an immortalized cell or a genetically modified cell derived from an immortalized cell. In an embodiment, the cell is a live cell, e.g., is viable as measured by any technique described herein or known in the art.

“Cell-binding peptide (CBP)”, as used herein, means a linear or cyclic peptide that comprises an amino acid sequence that is derived from the cell binding domain of a ligand for a cell-adhesion molecule (CAM) (e.g., that mediates cell-matrix junctions or cell-cell junctions). In an embodiment, the CBP is any of the CBPs described in international patent publication WO 2020/069429. In an embodiment, the CBP is a linear peptide comprising RGD and is less than 6 amino acids in length. In an embodiment, the CBP is a linear peptide that consists essentially of RGD or RGDSP.

“CBP-polymer”, as used herein, means a polymer comprising at least one cell-binding peptide molecule covalently attached to the polymer via a linker. In an embodiment, the polymer in a CBP-polymer is a synthetic or naturally-occurring polysaccharide, e.g., an alginate, e.g., a sodium alginate. In an embodiment, the linker is an amino acid linker (i.e., consists essentially of a single amino acid, or a peptide of several identical or different amino acids), which is joined via a peptide bond to the N-terminus or C-terminus of the CBP. In an embodiment, the CBP-polymer is any of the CBP-alginates defined in WO 2020/069429.

“Cell-binding substance (CBS)”, as used herein, means any chemical, biological, or other type of substance (e.g., a small organic compound, a peptide, a polypeptide) that is capable of mimicking at least one activity of a ligand for a cell-adhesion molecule (CAM) or other cell-surface molecule that mediates cell-matrix junctions or cell-cell junctions or other receptor-mediated signaling. In an embodiment, when present in a polymer composition encapsulating live cells, the CBS is capable of forming a transient or permanent bond or contact with one or more of the cells. In an embodiment, the CBS facilitates interactions between two or more live cells encapsulated in the polymer composition. In an embodiment, the presence of a CBS in a polymer composition encapsulating a plurality of cells (e.g., live cells) is correlated with one or both of increased cell productivity (e.g., expression of a therapeutic agent) and increased cell viability when the encapsulated cells are implanted into a test subject, e.g., a mouse. In an embodiment, the CBS is physically attached to one or more polymer molecules in the polymer composition. In an embodiment, the CBS is a cell-binding peptide, as defined herein or in WO 2020/069429.

“Conservatively modified variants” or conservative substitution”, as used herein, refers to a variant of a reference peptide or polypeptide that is identical to the reference molecule, except for having one or more conservative amino acid substitutions in its amino acid sequence. In an embodiment, a conservatively modified variant consists of an amino acid sequence that is at least 70%, 80%, 85%, 90%, 95%, 97% 98% or 99% identical to the reference amino acid sequence. A conservative amino acid substitution refers to substitution of an amino acid with an amino acid having similar characteristics (e.g., charge, side-chain size, hydrophobicity/hydrophilicity, backbone conformation and rigidity, etc.) and which has minimal impact on the biological activity of the resulting substituted peptide or polypeptide. Conservative substitution tables of functionally similar amino acids are well known in the art, and exemplary substitutions grouped by functional features are set forth in Table 1 below.

TABLE 1
Exemplary conservative amino acid substitution groups.

| Feature | Conservative Amino Group |
|---|---|
| Charge/Polarity | His, Arg, Lys |
| | Asp, Glu |
| | Cys, Thr, Ser, Gly, Asn, Gln, Tyr |
| | Ala, Pro, Met, Leu, Ile, Val, Phe, Trp |
| Hydrophobicity | Asp, Glu, Asn, Gln, Arg, Lys |
| | Cys, Ser, Thr, Pro, Gly, His, Tyr |
| | Ala, Met, Ile Leu, Val, Phe, Trp |
| Structural/Surface | Asp, Glu, Asn, Aln, His, Arg, Lys |
| Exposure | Cys, Ser, Tyr, Pro, Ala, Gly, Trp, Tyr |
| | Met, Ile, Leu, Val, Phe |
| Secondary Structure | Ala, Glu, Aln, His, Lys, Met, Leu, Arg |
| Propensity | Cys, Thr, Ile, Val, Phe, Tyr, Trp |
| | Ser, Gly, Pro, Asp, Asn |
| Evolutionary | Asp, Glu |
| Conservation | His, Lys, Arg |
| | Asn, Gln |
| | Ser, Thr |
| | Leu, Ile, Val |
| | Phe, Tyr, Trp |
| | Ala, Gly |
| | Met, Cys |

“Consists essentially of”, and variations such as “consist essentially of” or “consisting essentially of” as used throughout the specification and claims, indicate the inclusion of any recited elements or group of elements, and the optional inclusion of other elements, of similar or different nature than the recited elements, that do not materially change the basic or novel properties of the specified molecule, composition, hydrogel capsule, or method. As a non-limiting example, a therapeutic substance that consists essentially of a recited amino acid sequence may also include one or more amino acids, including substitutions in the recited amino acid sequence, of one or more amino acid residues, which do not materially affect the relevant biological activity of the therapeutic substance.

“Derived from”, as used herein with respect to cells, refers to cells obtained from tissue, cell lines, or cells, which optionally are then cultured, passaged, differentiated, induced, etc. to produce the derived cells. For example, mesenchymal stem cells can be derived from mesenchymal tissue and then differentiated into a variety of cell types.

“Exogenous nucleic acid,” as used herein, is a nucleic acid that does not occur naturally in a subject cell.

“Exogenous polypeptide,” as used herein, is a polypeptide encoded by an exogenous nucleic acid introduced into the cell. Reference to an amino acid position of a specific sequence means the position of said amino acid in a reference amino acid sequence, e.g., sequence of a full-length mature (after signal peptide cleavage) wild-type protein (unless otherwise stated), and does not exclude the presence of variations, e.g., deletions, insertions and/or substitutions at other positions in the reference amino acid sequence.

“Factor VII protein” or “FVII protein” as used herein, means a polypeptide that comprises the amino acid sequence of a naturally occurring factor VII protein or variant thereof that has a FVII biological activity, e.g., promoting blood clotting, as determined by an art-recognized assay, unless otherwise specified. Naturally occurring FVII exists as a single chain zymogen, a zymogen-like two-chain polypeptide and a fully activated two-chain form (FVIIa). In some embodiments, reference to FVII includes single-chain and two-chain forms thereof, including zymogen-like and FVIIa. FVII proteins that may be produced by a genetically modified cell described herein (e.g., derived from a human epithelial cell line, e.g., the ARPE-19 cell line), include wild-type primate (e.g., human), porcine, canine, and murine proteins, as well as variants of such wild-type proteins, including fragments, mutants, variants with one or more amino acid substitutions and/or deletions. In some embodiments, a variant FVII protein is capable of being activated to the fully activated two-chain form (Factor VIIa) that has at least 50%, 75%, 90% or more (including >100%) of the activity of wild-type Factor VIIa. Variants of FVII and FVIIa are known, e.g., marzeptacog alfa (activated) (MarzAA) and the variants described in European Patent No. 1373493, U.S. Pat. Nos. 7,771,996, 9,476,037 and US published application No. US 2008/0058255.

Factor VII biological activity may be quantified by an art recognized assay, unless otherwise specified. For example, FVII biological activity in a sample of a biological fluid, e.g., plasma, may be quantified by (i) measuring the amount of Factor Xa produced in a system comprising tissue factor (TF) embedded in a lipid membrane and Factor X (Persson et al., J. Biol. Chem. 272:19919-19924, 1997); (ii) measuring Factor X hydrolysis in an aqueous system; (iii) measuring its physical binding to TF using an instrument based on surface plasmon resonance (Persson, FEBS Letts. 413:359-363, 1997); or (iv) measuring hydrolysis of a synthetic substrate; and/or (v) measuring generation of thrombin in a TF-independent in vitro system. In an embodiment, FVII activity is assessed by a commercially available chromogenic assay (BIOPHEN FVII, HYPHEN BioMed Neuville sur Oise, France), in which the biological sample containing FVII is mixed with thromboplastin calcium, Factor X and SXa-11 (a chromogenic substrate specific for Factor Xa.

“Factor VIII protein” or “FVIII protein” as used herein, means a polypeptide that comprises the amino acid sequence of a naturally occurring factor VIII polypeptide or variant thereof that has an FVIII biological activity, e.g., coagulation activity, as determined by an art-recognized assay, unless otherwise specified. FVIII proteins that may be expressed by a genetically modified cell described herein (e.g., derived from a human epithelial cell line, e.g., the ARPE-19 cell line), include wild-type primate (e.g., human), porcine, canine, and murine proteins, as well as variants of such wild-type proteins, including fragments, mutants, variants with one or more amino acid substitutions and/or deletions, B-domain deletion (BDD) variants, single chain variants and fusions of any of the foregoing wild-type or variants with a half-life extending polypeptide. In an embodiment, the cells comprise an exogenous sequence that encodes a precursor factor VIII polypeptide (e.g., with the signal sequence) with a full or partial deletion of the B domain. In an embodiment, the cells comprise an exogenous sequence that encodes a single chain factor VIII polypeptide. In an embodiment, the expressed FVIII protein is a variant FVIII protein that has at least 50%, 75%, 90% or more (including >100%) of the coagulation activity of the corresponding wild-type factor VIII, e.g., human wild-type FVIII. Assays for measuring the coagulation activity of FVIII proteins include the one stage or two stage coagulation assay (Rizza et al., 1982, Coagulation assay of FVIII:C and FIXa in Bloom ed. The Hemophelias. NY Churchill Livingston 1992) or the chromogenic substrate FVIII:C assay (Rosen, S. 1984. Scand J Haematol 33:139-145, suppl.).

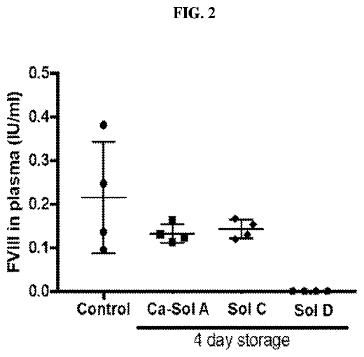

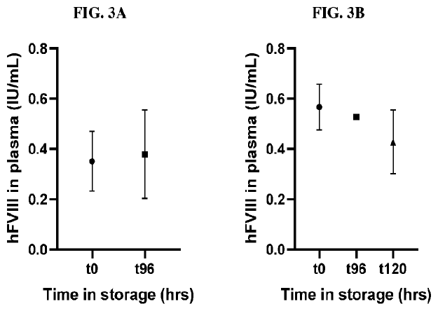

A number of FVIII-BDD variants are known, and include, e.g., variants with the full or partial B-domain deletions disclosed in any of the following U.S. Pat. No. 4,868,112 (e.g., col. 2, line 2 to col. 19, line 21 and table 2); U.S. Pat. No. 5,112,950 (e.g., col. 2, lines 55-68, FIG. 2, and example 1); U.S. Pat. No. 5,171,844 (e.g., col. 4, line 22 to col. 5, line 36); U.S. Pat. No. 5,543,502 (e.g., col. 2, lines 17-46); U.S. Pat. Nos. 5,595,886; 5,610,278; 5,789,203 (e.g., col. 2, lines 26-51 and examples 5-8); U.S. Pat. No. 5,972,885 (e.g., col. 1, lines 25 to col. 2, line 40); U.S. Pat. No. 6,048,720 (e.g., col. 6, lines 1-22 and example 1); U.S. Pat. Nos. 6,060,447; 6,228,620; 6,316,226 (e.g., col. 4, line 4 to col. 5, line 28 and examples 1-5); U.S. Pat. Nos. 6,346,513; 6,458,563 (e.g., col. 4, lines 25-53) and U.S. Pat. No. 7,041,635 (e.g., col. 2, line 1 to col. 3, line 19, col. 3, line 40 to col. 4, line 67, col. 7, line 43 to col. 8, line 26, and col. 11, line 5 to col. 13, line 39). In an embodiment, the encapsulated cells comprise an exogenous nucleotide sequence that encodes the mature FVII-BDD amino acid sequence shown in FIG. 3 of WO 2019/067766.

In some embodiments, a FVIII-BDD protein produced by a genetically modified cell described herein (e.g., derived from a human epithelial cell line, e.g., the ARPE-19 cell line) has one or more of the following deletions of amino acids in the B-domain: (i) most of the B domain except for amino-terminal B-domain sequences essential for intracellular processing of the primary translation product into two polypeptide chains (WO 91/09122); (ii) a deletion of amino acids 747-1638 (Hoeben R. C., et al. J. Biol. Chem. 265 (13): 7318-7323 (1990)); amino acids 771-1666 or amino acids 868-1562 (Meulien P., et al. Protein Eng. 2(4):301-6 (1988); amino acids 982-1562 or 760-1639 (Toole et al., Proc. Natl. Acad. Sci. U.S.A. 83:5939-5942 (1986)); amino acids 797-1562 (Eaton et al., Biochemistry 25:8343-8347 (1986)); 741-1646 (Kaufman, WO 87/04187)), 747-1560 (Sarver et al., DNA 6:553-564 (1987)); amino acids 741-1648 (Pasek, WO 88/00831)), amino acids 816-1598 or 741-1689 (Lagner (Behring Inst. Mitt. (1988) No 82:16-25, EP 295597); a deletion that includes one or more residues in a furin protease recognition sequence, including any of the specific deletions recited in U.S. Pat. No. 9,956,269 at col. 10, line 65 to col. 11, line 36.

In other embodiments, a FVIII-BDD protein retains any of the following B-domain amino acids or amino acid sequences: (i) one or more N-linked glycosylation sites in the B-domain, e.g., residues 757, 784, 828, 900, 963, or optionally 943, first 226 amino acids or first 163 amino acids (Miao, H. Z., et al., Blood 103(a): 3412-3419 (2004), Kasuda, A., et al., J. Thromb. Haemost. 6: 1352-1359 (2008), and Pipe, S. W., et al., J. Thromb. Haemost. 9: 2235-2242 (2011).

In some embodiments, the FVIII-BDD protein is a single-chain variant generated by substitution or deletion of one or more amino acids in the furin protease recognition sequence LKRHQR that prevents proteolytic cleavage at this site, including any of the substitutions at the R1645 and/or R1648 positions described in U.S. Pat. Nos. 10,023,628, 9,394,353 and 9,670,267.

In some embodiments, any of the above FVIII-BDD proteins may further comprise one or more of the following variations: a F309S substitution to improve expression of the FVIII-BDD protein (Miao, H. Z., et al., Blood 103(a): 3412-3419 (2004); albumin fusions (WO 2011/020866); and Fc fusions (WO 04/101740).

All FVIII-BDD amino acid positions referenced herein refer to the positions in full-length human FVIII, unless otherwise specified.

“Factor IX protein” or “FIX protein”, as used herein, means a polypeptide that comprises the amino acid sequence of a naturally occurring factor IX protein or variant thereof that has a FIX biological activity, e.g., coagulation activity, as determined by an art-recognized assay, unless otherwise specified. FIX is produced as an inactive zymogen, which is converted to an active form by factor XIa excision of the activation peptide to produce a heavy chain and a light chain held together by one or more disulfide bonds. FIX proteins that may be produced by a genetically modified described herein (e.g., derived from an RPE cell line, e.g., the ARPE-19 cell line), include wild-type primate (e.g., human), porcine, canine, and murine proteins, as well as variants of such wild-type proteins, including fragments, mutants, variants with one or more amino acid substitutions and/or deletions and fusions of any of the foregoing wild-type or variant proteins with a half-life extending polypeptide. In an embodiment, cells are engineered to encode a full-length wild-type human factor IX polypeptide (e.g., with the signal sequence) or a functional variant thereof. A variant FIX protein preferably has at least 50%, 75%, 90% or more (including >100%) of the coagulation activity of wild-type factor VIX. Assays for measuring the coagulation activity of FIX proteins include the Biophen Factor IX assay (Hyphen BioMed) and the one stage clotting assay (activated partial thromboplastin time (aPTT), e.g., as described in EP 2 032 607, thrombin generation time assay (TGA) and rotational thromboelastometry, e.g., as described in WO 2012/006624.

A number of functional FIX variants are known and may be expressed by engineered cells encapsulated in a device described herein, including any of the functional FIX variants described in the following international patent publications: WO 02/040544 at page 4, lines 9-30 and page 15, lines 6-31; WO 03/020764 in Tables 2 and 3 at pages 14-24, and at page 12, lines 1-27; WO 2007/149406 at page 4, line 1 to page 19, line 11; WO 2007/149406 A2 at page 19, line 12 to page 20, line 9; WO 08/118507 at page 5, line 14 to page 6, line 5; WO 09/051717 at page 9, line 11 to page 20, line 2; WO 09/137254 at page 2, paragraph [006] to page 5, paragraph [011] and page 16, paragraph [044] to page 24, paragraph [057]; WO 09/130198 A2 at page 4, line 26 to page 12, line 6; WO 09/140015 at page 11, paragraph [0043] to page 13, paragraph [0053]; WO 2012/006624; WO 2015/086406.

In certain embodiments, the FIX polypeptide comprises a wild-type or variant sequence fused to a heterologous polypeptide or non-polypeptide moiety extending the half-life of the FIX protein. Exemplary half-life extending moieties include Fc, albumin, a PAS sequence, transferrin, CTP (28 amino acid C-terminal peptide (CTP) of human chorionic gonadotropin (hCG) with its 4 O-glycans), polyethylene glycol (PEG), hydroxyethyl starch (HES), albumin binding polypeptide, albumin-binding small molecules, or any combination thereof. An exemplary FIX polypeptide is the rFIXFc protein described in WO 2012/006624, which is an FIXFc single chain (FIXFc-sc) and an Fc single chain (Fc-sc) bound together through two disulfide bonds in the hinge region of Fc.

FIX variants also include gain and loss of function variants. An example of a gain of function variant is the “Padua” variant of human FIX, which has a L (leucine) at position 338 of the mature protein instead of an R (arginine) (corresponding to amino acid position 384 of SEQ ID NO:20), and has greater catalytic and coagulant activity compared to wild-type human FIX (Chang et al., J. Biol. Chem., 273:12089-94 (1998)). An example of a loss of function variant is an alanine substituted for lysine in the fifth amino acid position from the beginning of the mature protein, which results in a protein with reduced binding to collagen IV (e.g., loss of function).

“Islet cell”, as used herein, means any cell that is naturally occurring or synthetically created, or modified, and is intended to recapitulate, mimic or otherwise express, in part or in whole, the functions, in part or in whole, of the cells of the pancreatic islets of Langerhans. The term “islet cell” includes a glucose-responsive, insulin producing cell derived from a stem cell, e.g., from an induced pluripotent stem cell line.

“Genetically-modified cell,” as used herein, is a cell (e.g., an RPE cell) having a non-naturally occurring alteration, and typically comprises a nucleic acid sequence (e.g., an exogenous DNA or RNA) or a polypeptide not present (or present at a different level than) in an otherwise similar cell under similar conditions that is not genetically modified (e.g., lacks the exogenous nucleic acid sequence). In an embodiment, a genetically modified cell comprises an exogenous nucleic acid (e.g., a vector or an altered chromosomal sequence). In an embodiment, a genetically modified cell comprises an exogenous polypeptide. In an embodiment, a genetically modified cell comprises an exogenous nucleic acid sequence, e.g., a sequence, e.g., DNA or RNA, not present in a similar cell that is not genetically modified. In an embodiment, the exogenous nucleic acid sequence is chromosomal, e.g., the exogenous nucleic acid sequence is an exogenous sequence disposed in endogenous chromosomal sequence. In an embodiment, the exogenous nucleic acid sequence is chromosomal or extra chromosomal, e.g., a non-integrated vector. In an embodiment, the exogenous nucleic acid sequence comprises an RNA sequence, e.g., an mRNA. In an embodiment, the exogenous nucleic acid sequence comprises a chromosomal or extra-chromosomal exogenous nucleic acid sequence that comprises a sequence which is expressed as RNA, e.g., mRNA or a regulatory RNA. In an embodiment, the exogenous nucleic acid sequence comprises a chromosomal or extra-chromosomal nucleic acid sequence, which comprises a sequence that encodes a polypeptide, or which is expressed as a polypeptide. In an embodiment, the exogenous nucleic acid sequence comprises a first chromosomal or extra-chromosomal exogenous nucleic acid sequence that modulates the conformation or expression of a second nucleic acid sequence, wherein the second amino acid sequence can be exogenous or endogenous. For example, a genetically modified cell can comprise an exogenous nucleic acid that controls the expression of an endogenous sequence. In an embodiment, a genetically modified cell comprises a polypeptide present at a level or distribution which differs from the level found in a similar cell that has not been genetically modified. In an embodiment, a genetically modified cell comprises an RPE genetically modified to produce an RNA or a polypeptide. For example, a genetically modified cell may comprise an exogenous nucleic acid sequence comprising a chromosomal or extra-chromosomal exogenous nucleic acid sequence that comprises a sequence which is expressed as RNA, e.g., mRNA or a regulatory RNA. In an embodiment, a genetically modified cell (e.g., an RPE cell) comprises an exogenous nucleic acid sequence that comprises a chromosomal or extra-chromosomal nucleic acid sequence comprising a sequence that encodes a polypeptide, or which is expressed as a polypeptide. In an embodiment, the polypeptide is encoded by a codon optimized sequence to achieve higher expression of the polypeptide than a naturally-occurring coding sequence. The codon optimized sequence may be generated using a commercially available algorithm, e.g., GeneOptimizer (ThermoFisher Scientific), OptimumGene™ (GenScript, Piscataway, NJ USA), GeneGPS® (ATUM, Newark, CA USA), or Java Codon Adaptation Tool (JCat, www.jcat.de, Grote, A. et al., Nucleic Acids Research, Vol 33, Issue suppl_2, pp. W526-W531 (2005)). In an embodiment, a genetically modified cell (e.g., an RPE cell) comprises an exogenous nucleic acid sequence that modulates the conformation or expression of an endogenous sequence. In an embodiment, a genetically modified cell (e.g., RPE cell) is cultured from a population of stably-transfected cells, or from a monoclonal cell line. “Peptide”, as used herein, is a polypeptide of less than 50 amino acids, typically, less than 25 amino acids.

“Iduronate-2-sulfatase protein”, “IDS protein”, “12S protein”, and “Alpha-L-iduronate sulfate sulfatase protein” may be used interchangeably herein to refer to a protein that comprises the mature amino acid sequence encoded by a wild-type mammalian (e.g., human) IDS gene or any fragment, mutant, variant or derivative thereof that has IDS enzyme activity that is within 80-120%, 85-115%, 90-110% or 95-105% of the corresponding wild-type mammalian mature IDS protein, as measured by any art-recognized IDS assay. IDS hydrolyzes the 2-sulfate groups of the L-iduronate 2-sulfate units of dermatan sulfate, beparan sulfate and heparan. The wild-type human IDS gene encodes a 550 amino acid precursor pro-polypeptide, of which the N-terminal 25 amino acids constitute a signal peptide, and the remaining amino acids constitute a pro-polypeptide that is processed into the mature polypeptide by removal of the pro-peptide of amino acids 26-33 and then cleavage into two chains formed by amino acids 34-455 and amino acids 456-550. (UniProtKB—P22304). In an embodiment, the GBA protein is part of a fusion protein that further comprises one or more amino acids sequences from one or more heterologous polypeptides.

“Osmolality” and “mOsm” are used herein to refer to a measure of the osmotic pressure of dissolved solute particles in an aqueous solution. The solute particles include both ions and non-ionized molecules. Osmolaltiy is typically expressed as the concentration of osmotically active particles (i.e., osmoles) dissolved in 1 kg of solution. “Osmolarity,” by contrast, refers to the number of solute particles dissolved in 1 liter of solution. For an aqueous solution, osmolarity is temperature dependent because water changes its volume with temperature. Therefore, osmolality is the preferred measure for an aqueous solution because it is not temperature dependent. If the concentration of solutes is very low, osmolarity and osmolality are considered equivalent. When used herein, the abbreviation “mOsm” means “milliosmoles/kg solution”.

“Peptide”, as used herein, is a polypeptide of less than 50 amino acids, typically, less than 25 amino acids.

“PolyA” signal, as used herein, refers to any continuous sequence of adenylic acids that terminates transcription of a coding sequence into RNA and directs addition of a polyA tail onto the RNA. The length of a polyA sequence is from 10- to 200 nucleotides and may be controlled variously depending on the allowable size of the backbone of the expression vector. Examples of polyA signals are the rabbit binding globulin (rBG) polyA signal, the SV40 late poly A signal, the SV50 polyA signal, the bovine growth hormone (BGH) poly A signal, the human growth hormone (HGH) polyA signal and synthetic polyA signals known in the art.

“Polymer composition”, as used herein, is a composition (e.g., a solution, mixture) comprising one or more polymers. As a class, “polymers’ includes homopolymers, heteropolymers, co-polymers, block polymers, block co-polymers and can be both natural and synthetic. Homopolymers contain one type of building block, or monomer, whereas co-polymers contain more than one type of monomer.

“Polypeptide”, as used herein, is a polymer comprising amino acid residues linked through peptide bonds and having at least two, and in some embodiments, at least 10, 50, 75, 100, 150 or 200 amino acid residues.

“Prevention,” “prevent,” and “preventing”, as used herein, refer to a treatment that comprises administering or applying a therapy, e.g., administering a hydrogel capsule composition described herein, prior to the onset of a disease, disorder, or condition to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

“Promoter sequence”, as used herein refers to a nucleotide sequence that is capable of driving expression in a mammalian cell, e.g., a human cell, e.g., an ARPE-19 cell. In some embodiments, the promoter sequence is from a strong mammalian promoter, e.g., a human promoter sequence. Non-limiting examples of strong promoters for use in genetically modified cells described herein include the EF-1 alpha (EF1A) promoter, CAG promoter, PGK (phosphoglycerate kinase) promoter and the ACTB (human beta-actin) promoter. In an embodiment, a promoter sequence may be from a medium-strength promoter, e.g., the EFS promoter sequence, which is a shortened form of the EF1A promoter sequence.

“Protein”, as used herein, comprises one or more polypeptide chains of at least 50 amino acids in length. In an embodiment, a protein has two or more polypeptide chains have identical or non-identical amino acid sequences of at least 50 amino acids in length. In an embodiment, the polypeptide chains in a protein are noncovalently associated or covalently joined, e.g., via disulfide bond(s).

“RPE cell”, as used herein, refers to a cell having one or more of the following characteristics: a) it comprises a retinal pigment epithelial cell (RPE) (e.g., cultured using the ARPE-19 cell line (ATCC® CRL-2302™)) or a cell derived therefrom, e.g., by stably transfecting cells cultured from the ARPE-19 cell line with an exogenous sequence that encodes an therapeutic substance or otherwise engineering such cultured ARPE-19 cells to express an exogenous protein or other exogenous substance, a cell derived from a primary cell culture of RPE cells, a cell isolated directly (without long term culturing, e.g., less than 5 or 10 passages or rounds of cell division since isolation) from naturally occurring RPE cells, e.g., from a human or other mammal, a cell derived from a transformed, an immortalized, or a long term (e.g., more than 5 or 10 passages or rounds of cell division) RPE cell culture; b) a cell that has been obtained from a less differentiated cell, e.g., a cell developed, programmed, or reprogramed (e.g., in vitro) into an RPE cell or a cell that is, except for any genetic engineering, substantially similar to one or more of a naturally occurring RPE cell or a cell from a primary or long term culture of RPE cells (e.g., the cell can be derived from an IPS cell); or c) a cell that has one or more of the following properties: i) it expresses one or more of the biomarkers CRALBP, RPE-65, RLBP, BEST1, or αB-crystallin; ii) it does not express one or more of the biomarkers CRALBP, RPE-65, RLBP, BEST1, or αB-crystallin; iii) it is naturally found in the retina and forms a monolayer above the choroidal blood vessels in the Bruch's membrane; iv) it is responsible for epithelial transport, light absorption, secretion, and immune modulation in the retina; or v) it has been created synthetically, or modified from a naturally occurring cell, to have the same or substantially the same genetic content, and optionally the same or substantially the same epigenetic content, as an immortalized RPE cell line (e.g., the ARPE-19 cell line (ATCC^{Ò} CRL-2302Ô)). In an embodiment, an RPE cell described herein is genetically modified, e.g., to have a new property, e.g., the cell is genetically modified to express and secrete one or more therapeutic substances. In other embodiments, an RPE cell is not genetically modified.

“Sequence identity” or “percent identical”, when used herein to refer to two nucleotide sequences or two amino acid sequences, means the two sequences are the same within a specified region, or have the same nucleotides or amino acids at a specified percentage of nucleotide or amino acid positions within the specified when the two sequences are compared and aligned for maximum correspondence over a comparison window or designated region. Sequence identity may be determined using standard techniques known in the art including, but not limited to, any of the algorithms described in US Patent Application Publication No. 2017/02334455. In an embodiment, the specified percentage of identical nucleotide or amino acid positions is at least about 80%, 85%, 90%, 91%, 92%, 93%, 94%, 95%, 96%, 97%, 98%, 99% or higher.

“Spherical” as used herein, means a hydrogel capsule having a curved surface that forms a sphere (e.g., a completely round ball) or sphere-like shape, which may have waves and undulations, e.g., on the surface. Spheres and sphere-like objects can be mathematically defined by rotation of circles, ellipses, or a combination around each of the three perpendicular axes, a, b, and c. For a sphere, the three axes are the same length. Generally, a sphere-like shape is an ellipsoid (for its averaged surface) with semi-principal axes within 10%, or 5%, or 2.5% of each other. The diameter of a sphere or sphere-like shape is the average diameter, such as the average of the semi-principal axes.

“Spheroid”, as that term is used herein to refer to a hydrogel capsule, means the capsule has (i) a perfect or classical oblate spheroid or prolate spheroid shape or (ii) has a surface that roughly forms a spheroid, e.g., may have waves and undulations and/or may be an ellipsoid (for its averaged surface) with semi-principal axes within 100% of each other.

“Subject” as used herein refers to a human or non-human animal. In an embodiment, the subject is a human (i.e., a male or female), e.g., of any age group, a pediatric subject (e.g., infant, child, adolescent) or adult subject (e.g., young adult, middle-aged adult, or senior adult). In an embodiment, the subject is a non-human animal, for example, a mammal (e.g., a mouse, a dog, a primate (e.g., a cynomolgus monkey or a rhesus monkey)). In an embodiment, the subject is a commercially relevant mammal (e.g., a cattle, pig, horse, sheep, goat, cat, or dog) or a bird (e.g., a commercially relevant bird such as a chicken, duck, goose, or turkey). In certain embodiments, the animal is a mammal. The animal may be a male or female and at any stage of development. A non-human animal may be a transgenic animal.

“Transcription unit” means a DNA sequence, e.g., present in an exogenous nucleic acid, that comprises at least a promoter sequence operably linked to a coding sequence, and may also comprise one or more additional elements that control or enhance transcription of the coding sequence into RNA molecules or translation of the RNA molecules into polypeptide molecules. In some embodiments, a transcription unit also comprises a polyadenylation (polyA) signal sequence and polyA site.

“Treatment,” “treat,” and “treating” as used herein refers to one or more of reducing, reversing, alleviating, delaying the onset of, or inhibiting the progress of one or more of a symptom, manifestation, or underlying cause, of a disease, disorder, or condition. In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, a therapy (e.g., a hydrogel capsule composition) may be administered to a susceptible individual prior to the onset of symptoms (e.g., considering a history of symptoms and/or in light of genetic or other susceptibility factors). Treatment may also be continued after symptoms have resolved, for example, to delay or prevent recurrence. In some embodiments, treatment comprises prevention and in other embodiments it does not.

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”), 1 to 10 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”), 2 to 8 carbon atoms (“C₂-C₈ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”), 2 to 8 carbon atoms (“C₂-C₈ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂-C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. An alkylene, alkenylene, alkynylene, or heteroalkylene group may be described as, e.g., a C₁-C₆-membered alkylene, C₂-C₆-membered alkenylene, C₂-C₆-membered alkynylene, or C₁-C₆-membered heteroalkylene, wherein the term “membered” refers to the non-hydrogen atoms within the moiety. In the case of heteroalkylene groups, heteroatoms can also occupy either or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, the terms “arylene” and “heteroarylene,” alone or as part of another substituent, mean a divalent radical derived from an aryl and heteroaryl, respectively.

As used herein, “cycloalkyl” refers to a radical of a non-aromatic cyclic hydrocarbon group having from 3 to 10 ring carbon atoms (“C₃-C₁₀ cycloalkyl”) and 0 heteroatoms in the non-aromatic ring system. In some embodiments, a cycloalkyl group has 3 to 8 ring carbon atoms (“C₃-Cgcycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro [4.5] decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, piperazinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl or thiomorpholinyl-1,1-dioxide.

Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Amino” as used herein refers to the radical —NR⁷⁰R⁷¹, wherein R⁷⁰ and R⁷¹ are each independently hydrogen, C₁-C₈ alkyl, C₃-C₁₀ cycloalkyl, C₄-C₁₀ heterocyclyl, C₆-C₁₀ aryl, and C₅-C₁₀ heteroaryl. In some embodiments, amino refers to NH₂.

As used herein, “cyano” refers to the radical —CN.

As used herein, “halo” or “halogen,” independently or as part of another substituent, mean, unless otherwise stated, a fluorine (F), chlorine (Cl), bromine (Br), or iodine (I) atom.

As used herein, “hydroxy” refers to the radical —OH.

Alkyl, alkenyl, alkynyl, heteroalkyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations to arrive at a stable compound. For purposes of this disclosure, heteroatoms such as nitrogen may have hydrogen substituents and/or any suitable substituent as described herein that satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds of Formula (I) described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, e.g., Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

Compounds of Formula (I) described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The term “pharmaceutically acceptable salt” is meant to include salts of the active compounds that are prepared with relatively nontoxic acids or bases, depending on the particular substituents found on the compounds described herein. When compounds of Formula (I) used to prepare hydrogel capsules of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al, Journal of Pharmaceutical Science 66: 1-19 (1977)). Certain specific compounds used in the hydrogel capsules of the present disclosure contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

Hydrogel capsules in a composition of the present disclosure, e.g., in a population of hydrogel capsules in the composition, may contain a compound of Formula (I) in a prodrug form. Prodrugs are those compounds that readily undergo chemical changes under physiological conditions to provide the compounds useful for preparing capsules in the present disclosure.

Additionally, prodrugs can be converted to useful compounds of Formula (I) by chemical or biochemical methods in an ex vivo environment.

Certain compounds of Formula (I) described herein can exist in unsolvated forms as well as solvated forms, including hydrated forms. In general, the solvated forms are equivalent to unsolvated forms and are encompassed within the scope of the present disclosure. Certain compounds of Formula (I) described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, e.g., by the general formula R×x H₂O, wherein R is the compound and wherein x is a number greater than 0.

The symbol “ [symbol] ” as used herein refers to a connection to an entity, e.g., a polymer (e.g., hydrogel-forming polymer such as alginate) or surface of a hydrogel capsule. The connection represented by “ [symbol] ” may refer to direct attachment to the entity, e.g., a polymer or capsule surface, may refer to linkage to the entity through an attachment group. An “attachment group,” as described herein, refers to a moiety for linkage of a compound of Formula (I) to an entity (e.g., a polymer or a hydrogel capsule as described herein), and may comprise any attachment chemistry known in the art. A listing of exemplary attachment groups is outlined in Bioconjugate Techniques (3^{rd} ed, Greg T. Hermanson, Waltham, M A: Elsevier, Inc, 2013), which is incorporated herein by reference in its entirety. In some embodiments, an attachment group comprises alkyl, alkenyl, alkynyl, heteroalkyl, cycloalkyl, heterocyclyl, aryl, heteroaryl, —C(O)—, —OC(O)—, —N(R^{C})—, —N(R^{C})C(O)—, —C(O)N(R^{C})—, —N(R^{C})N(R^{D})—, —NCN—, —C(═N(R^{C})(R^{D}))O, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A}), —B(OR^{A}), or a metal, wherein each of R^{A}, R^{C}, R^{D}, R^{F}, R^{G}, x and y is independently as described herein. In some embodiments, an attachment group comprises an amine, ketone, ester, amide, alky. In some embodiments, an attachment group is a cross-linker. In some embodiments, the attachment group is —C(O)(C₁-C₆¹, and R¹ is as described herein. In some embodiments, the attachment group is —C(O)(C₁-C₆-alkylene)-, wherein alkylene is substituted with 1-2 alkyl groups (e.g., 1-2 methyl groups). In some embodiments, the attachment group is —C(O)C(CH₃)₂—. In some embodiments, the attachment group is —C(O)(methylene)-, wherein alkylene is substituted with 1-2 alkyl groups (e.g., 1-2 methyl groups). In some embodiments, the attachment group is —C(O)CH(CH₃)—. In some embodiments, the attachment group is —C(O)C(CH₃)—.

### Features of Hydrogel Capsule Compositions

A hydrogel capsule composition of the present disclosure comprises a population of hydrogel capsules disposed in a pharmaceutically acceptable aqueous solution. The hydrogel capsules in the population encapsulate cells (e.g., mammalian cells) and comprise a hydrogel-forming polymer, e.g., a naturally-occurring or synthetic polysaccharide. Exemplary polysaccharides include, e.g., alginate, agar, agarose, carrageenans, cellulose and amylose, chitin, chitosan and hyaluronate. The hydrogel-forming polymer can be cross-linked, e.g., cross-linked by diacrylates or can comprise a polysaccharide or derivative/modification thereof described in, e.g., Laurienzo (2010), Mar. Drugs. 8.9:2435-65. In an embodiment, the capsules comprise a mixture of two or more hydrogel-forming polymers, e.g., a mixture of two alginates having different G:M content and/or different average molecular weight. In an embodiment, the hydrogel capsules comprise ionically cross-linked alginate(s). (e.g., cross-linked with divalent cations, e.g., barium, calcium, magnesium or strontium. In an embodiment, the capsules in the population comprise an ionically cross-linked alginate.

The hydrogel capsules in the composition can have any of a variety of shapes: cylinder, cylinder with hemispherical ends (also known as spherocylinder), disc, noodle (e.g., as described in WO 2015/191547), sphere (e.g., as defined herein), or spheroid (e.g., as defined herein). In an embodiment, the hydrogel capsules are spheres, as defined herein.

In an embodiment, the composition includes one or more additional capsules that do not have all of the features of the population of capsules, e.g., the additional, non-population capsules may be composed of a different hydrogel, may be of a different shape or size, may encapsulate different cell types and or average number of cells, or may not encapsulate any cells.

### Aqueous Solution

In some embodiments, the solution in the hydrogel capsule composition comprises elemental calcium at a concentration of at least about 1.0 mM but no more than about 10 mM. In an embodiment, the elemental calcium concentration is less than about 6 mM. In an embodiment, the elemental calcium concentration is between about 1.2 mM and about 2.0 mM. In an embodiment, the elemental calcium concentration is between about 1.5 mM and about 2.5 mM. In an embodiment, the elemental calcium concentration is about 2.0 mM. The source of the calcium can be one of more of any pharmaceutically acceptable calcium salt, e.g., calcium acetate, calcium carbonate, calcium citrate, calcium chloride or calcium gluconate. In an embodiment, the calcium salt is calcium chloride.

Calcium can be measured by the amount of the calcium salt (mg of the cationplus the anion, or mL of a specified concentration) or the amount of elemental calcium in milligrams (mg), in milliequivalents (mEq), or in millimoles (mmol). Because calcium has a valence of +2, the mEq equals two times the number of mM. A calcium equivalents calculator is publicly available on the Cornell University Medical College website at http://www-users.med.cornell.edu/˜spon/picu/calc/cacalc.htm. The elemental Ca amounts in 1 g of common calcium salts are shown in Table 2 below.

TABLE 2
Elemental calcium in calcium salts.

| Salt | Elemental Ca | | | |
|---|---|---|---|---|
| Calcium Acetate | 1 g | 12.6 mEq | 253 mg | 6.30 mmol |
| Calcium Carbonate | 1 g | 19.9 mEq | 400 mg | 9.96 mmol |
| Calcium Citrate | 1 g | 10.5 mEq | 211 mg | 5.26 mmol |
| Calcium Chloride | 1 g | 13.6 mEq | 273 mg | 6.80 mmol |
| Calcium Glubionate | 1 g | 3.29 mEq | 66 mg | 1.64 mmol |
| Calcium Gluceptate | 1 g | 4.08 mEq | 82 mg | 2.04 mmol |
| Calcium Gluconate | 1 g | 4.65 mEq | 93 mg | 2.32 mmol |

In some embodiments, the aqueous solution has an osmolality of about 250 mOsm to about 350 mOsm, a pH of between 6.0 and 9.0 at 12° C. to 30° C. (e.g., about 15-25° C.) (e.g., between 6.5 and 9.0) and comprises a calcium salt at an elemental calcium concentration of between at least about 1.0 millimolar (mM) and about 10 mM. The components of the solution should be pharmaceutically acceptable for administration to a mammal (e.g., a human). In addition, the components of the solution are capable of supporting viability of the encapsulated cells.

Cell viability can be impacted by the osmolality of the aqueous solution. The osmolality of the solution can be maintained between the desired range by including in the solution appropriate amounts of one or more osmotically active agents known in the art, such as ions (e.g., sodium, potassium, chloride, bicarbonate, calcium, phosphate), monosaccharides (e.g., glucose, fructose), oligosaccharides (e.g., sucrose, lactose, dextrose, mannitol), amino acids and the like. The osmolality of the solution can be determined by methods known in the art using an osmometer, e.g., a freezing point depression osmometer. In an embodiment, the osmolality of the solution can be, e.g., 250, 260, 265, 270, 280, 290, 300, 310, 320, 330, 340, or 350 mOsm/kg, or any numerical value between about 250 and 350 mOsm/kg.

Since the pH of the aqueous solution can also impact cell viability and/or productivity, the pH is typically controlled between 6.5 to 9.0 in a temperature range of 12° C. to 30° C. by including one or more pharmaceutically acceptable buffering agents that have an appropriate pKa and are compatible with cell viability. Exemplary buffers that are suitable for inclusion in the aqueous solution include, but are not limited to bicarbonate salts, acetate salts, phosphate salts, gluconate salts, and lactate salts. In an embodiment, the buffering agent is not HEPES. In an embodiment, the buffering agent comprises sodium acetate and sodium gluconate. In an embodiment, the buffering agent comprises sodium bicarbonate and sodium phosphate.

In some embodiments, the aqueous solution in the hydrogel capsule composition may also include at least one carbon source to help support viability of the cells. In an embodiment, the carbon source may be provided by an osmotically active agent or buffering agent in the solution. In another embodiment, each carbon source is different than other components in the solution. Exemplary carbon sources include gluconate salts, sugars (e.g., dextrose, glucose, galactose, hexose, fructose, maltose), glycerol, glutamine and pyruvate (and pharmaceutically acceptable salts of pyruvate). In an embodiment, sodium gluconate is present in the solution to provide a carbon source, and may also act as a buffering agent. In an embodiment, the solution comprises glucose as a carbon source. In an embodiment, the solution comprises about 5 mM to about 25 mM D-glucose.

In an embodiment, the aqueous solution in a hydrogel capsule composition comprises, consists essentially of, or consists of calcium chloride, sodium chloride, sodium acetate, sodium gluconate, potassium chloride and magnesium chloride. In an embodiment, the solution consists essentially of the components listed in Table 3A or 3B below.

TABLE 3A
Exemplary Aqueous Solution for Hydrogel Capsule Composition

| Component | Concentration (g/L) |
|---|---|
| Calcium chloride dihydrate | About 0.298 |
| Sodium chloride | About 5.252 |
| Sodium acetate | About 2.291 |
| Sodium gluconate | About 5.175 |
| Potassium chloride | About 0.3703 |
| Magnesium chloride hexahydrate | About 0.302 |
| Water | Not applicable |

TABLE 3B
Exemplary Aqueous Solution for Hydrogel Capsule Composition

| Component | Concentration (mM) |
|---|---|
| Glycine | About 0.400 |
| L-Arginine hydrochloride | About 0.398 |
| L-Cystine dihydrochloride | About 0.201 |
| L-Histidine monohydrochloride monohydrate | About 0.200 |
| L-Isoleucine | About 0.802 |
| L-Leucine | About 0.802 |
| L-Lysine hydrochloride | About 0.798 |
| L-Methionine | About 0.201 |
| L-Phenylalanine | About 0.400 |
| L-Serine | About 0.400 |
| L-Threonine | About 0.798 |
| L-Tryptophan | About 0.078 |
| L-Tyrosine disodium salt dihydrate | About 0.398 |
| L-Valine | About 0.803 |
| Choline chloride | About 0.029 |
| D-Calcium pantothenate | About 0.008 |
| Folic acid | About 0.009 |
| Niacinamide | About 0.038 |
| Pyridoxine hydrochloride | About 0.018 |
| Riboflavin | About 0.001 |
| Thiamine hydrochloride | About 0.012 |
| i-Inositol | About 0.040 |
| Calcium chloride (anhyd.) | About 1.802 |
| Ferric nitrate nonahydrate | About 2.475 × 10⁻⁴ |
| Magnesium sulfate (anhyd.) | About 0.814 |
| Potassium chloride | About 5.333 |
| Sodium bicarbonate | About 44.048 |
| Sodium chloride | About 110.345 |
| Sodium phosphate monobasic | About 0.906 |
| Water | Not applicable |

In an embodiment, the solution of Table 3B further comprises D-glucose in an amount selected from the groups consisting of: about 1 mM to about 50 mM; about 2 mM to about 40 mM; about 3 mM to about 30 mM; about 4 mM to about 20 mM; about 5 mM to about 10 mM; about 10 mM to about 40 mM; about 15 mM to about 35 mM; about 20 mM to about 30 mM; and about 25 mM.

### Hydrogel Capsules

The composition comprises a population of hydrogel capsules encapsulating cells. The hydrogel capsules in the population all have a substantially similar composition (e.g., alginate(s) and any other polymers used in the hydrogel) and configuration (e.g., shape, permeability, number of cell-containing compartments) that allows for retention of the cells inside the capsules and prevent entry of immune cells while allowing the passage of nutrients into the capsules and exit from the capsules of cellular waste materials and therapeutic substance(s) produced by the cells. Exemplary capsules encapsulating cells are described in U.S. Pat. Nos. 9,867,781; 10,292,936; 10,786,446; 10,898,443; and PCT International Publication Nos: WO 2019/169245; WO 2019/195055; WO 2021/113751; and WO2021/113751.

In an embodiment, each capsule in the population of hydrogel capsules is configured as a two-compartment hydrogel capsule in which an inner compartment comprises a first alginate hydrogel that encapsulates the cells and an outer barrier hydrogel compartment (also referred to herein as an outer layer) that completely surrounds the inner compartment and is substantially free of the encapsulated cells. The barrier compartment comprises an ionically cross-linked alginate hydrogel, which optionally comprises a mixture of two or more alginates having different average molecular weights.

In an embodiment, the inner boundary of the barrier compartment forms an interface with the outer boundary of the inner compartment. In such embodiments, the thickness of the barrier compartment means the average distance between the outer boundary of the barrier compartment and the interface between the two compartments, e.g., the average of the distances measured at each of the thinnest and thickest points visually observed in the barrier compartment. In some embodiments (e.g., the hydrogel capsule is about 1.5 mm in diameter), the thinnest and thickest distances for the barrier compartment are between 25 and 110 μm and between 270 and 480 μm, respectively. In some embodiments, the thickness of the barrier compartment is greater than about 10 nm, preferably 100 nm or greater and can be as large as 1 mm. For example, the thickness (e.g., average distance) of the barrier compartment in hydrogel capsules described herein may be 10 nm to 1 mm, 100 nm to 1 mm, 500 nm to 1 mm, 1 μm to 1 mm, 1 μm to 1 mm, 1 μm to 500 μm, 1 μm to 250 μm, 1 μm to 1 mm, 5 μm to 500 μm, 5 μm to 250 μm, 10 μm to 1 mm, 10 μm to 500 μm, or 10 μm to 250 μm. In some embodiments, the thickness (e.g., average distance) of the barrier compartment is 100 nm to 1 mm, between 1 μm and 1 mm, between 1 μm and 500 μm or between 5 μm and 1 mm. In some embodiments, the thickness (e.g., average distance) of the barrier compartment is between about 50 μm and about 100 μm. In some embodiments (e.g., the capsule is about 1.5 mm in diameter), the thickness of the barrier compartment (e.g., average distance) is between about 180 μm and 260 μm or between about 310 μm and 440 μm.

In some embodiments, the mean pore size of the cell-containing inner compartment and the cell-free barrier compartment is substantially the same. In some embodiments, the mean pore size of the inner compartment and the barrier compartment differ by about 1.5%, 2%, 5%, 7.5%, 10%, 15%, 20%, 25%, 30%, 35%, 40%, 45%, 50%, 55%, 60%, 65%, 70%, 75% or more. In some embodiments, the mean pore size of the capsule (e.g., mean pore size of the inner compartment and/or mean pore size of the barrier compartment) is dependent on a number of factors, such as the material(s) within each compartment and the presence and density of a compound of Formula (I).

In some embodiments, the mean capsule diameter or size of the two-compartment hydrogel capsules in the population is between any of the following ranges: about 0.5 mm to about 8 mm, about 0.5 mm to about 4 mm, about 0.5 mm to about 2 mm, about 0.7 mm to about 1.3 mm or about 1.2 mm to about 1.8 mm.

In some embodiments, the outer surface of each hydrogel capsule in the population comprises a compound capable of mitigating the FBR, e.g., an afibrotic compound of Formula (I) as described herein below, e.g., Compound 101. For capsules comprising a barrier compartment surrounding the cell-containing compartment, the afibrotic compound may be covalently attached to a polymer (e.g., an alginate) disposed throughout the barrier compartment. In an embodiment, some or all the monomers in the polymer are modified with the same compound of Formula (I). In some embodiments, some or all the monomers in the polymer are modified with different compounds of Formula (I).

In some embodiments, the alginate hydrogel in the barrier compartment comprises an afibrotic alginate, e.g, an alginate chemically modified with a Compound of Formula (I). The alginate in the modified alginate may be the same or different than any unmodified alginate that is present in the capsule. In an embodiment, the density of the Compound of Formula (I) in the modified alginate (e.g., amount of conjugation) is between about 4.0% and about 8.0%, between about 5.0% and about 7.0%, or between about 6.0% and about 7.0% nitrogen (e.g., as determined by combustion analysis for percent nitrogen). In an embodiment, the amount of Compound 101 produces an increase in % N (as compared with the unmodified alginate) of about 0.5% to 2% 2% to 4% N, about 4% to 6% N, about 6% to 8%, or about 8% to 10% N), where % N is determined by combustion analysis and corresponds to the amount of Compound 101 in the modified alginate.

In other embodiments, the density (e.g., concentration) of the Compound of Formula (I) (e.g., Compound 101) in the afibrotic alginate is defined as the % w/w, e.g., % of weight of amine/weight of afibrotic alginate in solution (e.g., saline) as determined by a suitable quantitative amine conjugation assay (e.g. by an assay described in WO 2020069429), and in certain embodiments, the density of a Compound of Formula (I) (e.g., Compound 101) is between about 1.0% w/w and about 3.0% w/w, between about 1.3% w/w and about 2.5% w/w or between about 1.5% w/w and 2.2% w/w.

The alginate in an afibrotic polymer can be chemically modified with a compound of Formula (I) using any suitable method known in the art. For example, the alginate carboxylic acid moiety can be activated for coupling to one or more amine-functionalized compounds to achieve an alginate modified with a compound of Formula (I). The alginate polymer may be dissolved in water (30 mL/gram polymer) and treated with 2-chloro-4,6-dimethoxy-1,3,5-triazine (0.5 eq) and N-methylmorpholine (1 eq). To this mixture may be added a solution of the compound of Formula (I) in acetonitrile (0.3M). The reaction may be warmed to 55° C. for 16 h, then cooled to room temperature and gently concentrated via rotary evaporation, then the residue may be dissolved, e.g., in water. The mixture may then be filtered, e.g., through a bed of cyano-modified silica gel (Silicycle) and the filter cake washed with water. The resulting solution may then be dialyzed (10,000 MWCO membrane) against water for 24 hours, e.g., replacing the water twice. The resulting solution can be concentrated, e.g., via lyophilization, to afford the desired chemically modified alginate.

The alginate hydrogel in the barrier compartment may also comprise an unmodified alginate. In some embodiments, the alginate is a high guluronic acid (G) alginate, and comprises greater than about 50%, 55%, 60%, 65%, 70%, 75%, 80%, 85%, 90%, or more guluronic acid (G). In some embodiments, the alginate is a high mannuronic acid (M) alginate, and comprises greater than about 50%, 55%, 60%, 65%, 70%, 75%, 80%, 85%, 90%, or more mannuronic acid (M). In some embodiments, the ratio of M:G is about 1. In some embodiments, the ratio of M:G is less than 1. In some embodiments, the ratio of M:G is greater than 1 or the ratio of G:M is greater than 1. In an embodiment, the unmodified alginate in the barrier compartment has a molecular weight of 150 kDa-250 kDa and a G:M ratio of ≥1.5.

In some embodiments, the alginate hydrogel in the inner compartment comprises an alginate in which the G:M ratio is greater than 1, e.g., a low molecular weight alginate (e.g., approximate molecular weight of <75 kD) and G:M ratio≥1.5, (ii) a medium molecular weight alginate, e.g., has approximate molecular weight of 75-150 kDa and G:M ratio≥1.5, (iii) a high molecular weight alginate, e.g., has an approximate MW of 150 kDa-250 kDa and G:M ratio≥1.5, (iv) or a blend of two or more of these alginates. In some embodiments, the inner compartment further comprises at least one cell-binding substance (CBS), e.g., a cell-binding peptide (CBP) or cell-binding polypeptide (CBPP) described in WO 2020069429.

In some embodiments, the inner compartment comprises an alginate covalently modified with a linker-cell-binding peptide moiety, e.g., GRGD or GRGDSP. In an embodiment, the cell-binding peptide density in the inner compartment (% nitrogen as determined by combustion analysis, e.g., as described in WO 2020198695) to be at least 0.05%, 0.1%, 0.2% or 0.3% but less than 4%, 3%, 2% or 1%. In an embodiment, the total density of the linker-CBP in the inner compartment is about 0.1 to about 1.0 micromoles of the CBP per g of CBP-alginate (e.g., a MMW-alginate covalently modified with GRGD or GRGDSP in solution as determined by a quantitative peptide conjugation assay, e.g., an assay described in WO 2020198695. In an embodiment, the linker-CBP is GRGDSP and the alginate has a molecular weight of 75 kDa to 150 kDa and a G:M ratio of greater than or equal to 1.5. In an embodiment, the inner compartment also comprises an unmodified alginate with a molecular weight of 75 kDa to 150 kDa and a G:M ratio of greater than or equal to 1.5.

In addition to an alginate hydrogel, either or both of the inner and outer compartments may further comprise a non-alginate polymer, which may be a linear, branched, or cross-linked polymer, or a polymer of selected molecular weight ranges, degree of polymerization, viscosity or melt flow rate. Branched polymers can include one or more of the following types: star polymers, comb polymers, brush polymers, dendronized polymers, ladders, and dendrimers. A non-alginate polymer may be a thermoresponsive polymer, e.g., gel (e.g., becomes a solid or liquid upon exposure to heat or a certain temperature) or a photocrosslinkable polymer. Exemplary polymers include polystyrene, polyethylene, polypropylene, polyacetylene, poly(vinyl chloride) (PVC), polyolefin copolymers, poly(urethane)s, polyacrylates and polymethacrylates, polyacrylamides and polymethacrylamides, poly(methyl methacrylate), poly(2-hydroxyethyl methacrylate), polyesters, polysiloxanes, polydimethylsiloxane (PDMS), polyethers such as polyether ketone (PEEK), poly(orthoester), poly(carbonates), poly(hydroxyalkanoate)s, polyfluorocarbons, polytetrafluoroethylene (PTFE), silicones, epoxy resins, polyethylene glycol, nylon, polyalkenes, phenolic resins, natural and synthetic elastomers, adhesives and sealants, polyolefins, polysulfones, polyacrylonitrile, biopolymers such as polysaccharides and natural latex, collagen, cellulosic polymers (e.g., alkyl celluloses, etc.), polyethylene glycol and 2-hydroxyethyl methacrylate (HEMA), polysaccharides, poly(glycolic acid), poly(L-lactic acid) (PLLA), poly(lactic glycolic acid) (PLGA), a polydioxanone (PDA), or racemic poly(lactic acid), polycarbonates, (e.g., polyamides (e.g., nylon)), fluoroplastics, carbon fiber, agarose, chitosan, and blends or copolymers thereof.

In some embodiments, each capsule in the population of hydrogel capsules comprises a plurality of cells (e.g., live cells) that are capable of expressing and secreting at least one therapeutic substance (e.g., a peptide or protein) when the hydrogel capsule is placed into a subject, e.g., as part of the entire volume of the capsule composition or an aliquot thereof. In some embodiments, the cells express two or more therapeutic substances, e.g., proteins with complementary activities useful for treating a particular disease of interest.

In an embodiment, each hydrogel capsule of the population comprises cells (e.g., in the inner compartment) that are derived from a single parental cell-type or a mixture of at least two different parental cell types. In an embodiment, all of the cells are derived from the same parental cell type, but a first plurality of the derived cells are genetically modified to express a first therapeutic substance, and a second plurality of the derived cells are genetically modified to express a second therapeutic substance. In hydrogel capsule compositions which comprise two or more populations of hydrogel capsules, the cells and the therapeutic substance(s) produced thereby may be the same or different for each capsule population.

In an embodiment, cells to be incorporated into a hydrogel capsule described herein are prepared in the form of a cell suspension prior to being encapsulated. The cells in the suspension may take the form of single cells (e.g., from a monolayer cell culture), or provided in another form, e.g., disposed on a microcarrier (e.g., a bead or matrix) or as a three-dimensional aggregate of cells (e.g., a cell cluster or spheroid). The cell suspension can comprise multiple cell clusters (e.g., as spheroids) or microcarriers.

In addition to therapeutic substance(s) expressed by the encapsulated cells, each hydrogel capsule of a capsule population may comprise one or more exogenous agents that are not expressed by the cells, and may include, e.g., a nucleic acid (e.g., an RNA or DNA molecule), a protein (e.g., a hormone, an enzyme (e.g., glucose oxidase, kinase, phosphatase, oxygenase, hydrogenase, reductase) antibody, antibody fragment, antigen, or epitope)), an active or inactive fragment of a protein or polypeptide, a small molecule, or drug. In an embodiment, the capsule is configured to release such an exogenous agent.

### Afibrotic Compounds

In some embodiments, the hydrogel capsules in a hydrogel capsule population described herein comprise a compound of Formula (I):

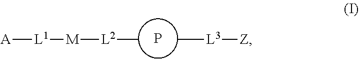

or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₂-C₆-alkenylene)-, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹;
each of L¹ and L³ is independently a bond, alkyl, or heteroalkyl, wherein each alkyl and heteroalkyl is optionally substituted by one or more R²
L² is a bond;
M is absent, alkyl, heteroalkyl, cycloalkyl, heterocyclyl, aryl, or heteroaryl, each of which is optionally substituted by one or more R³;
P is absent, cycloalkyl, heterocyclyl, or heteroaryl, each of which is optionally substituted by one or more R⁴;
Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

In some embodiments, the compound of Formula (I) is a compound of Formula (I-a):

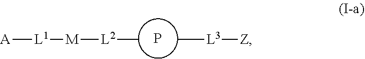

or a salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D})—, —NCN—, —N(R^{C})C(O)(C₁-C₆— alkylene)-, —N(R^{C})C(O)(C₂-C₆-alkenylene)-, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is heteroaryl optionally substituted by one or more R⁴;
Z is alkyl, alkenyl, alkynyl, heteroalkyl, cycloalkyl, heterocyclyl, aryl, or heteroaryl, each of which is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

In some embodiments, for Formulas (I) or (I-a), A is alkyl, alkenyl, alkynyl, heteroalkyl, cycloalkyl, heterocyclyl, aryl, heteroaryl, —O—, —C(O)O—, —C(O)—, —OC(O)—, —N(R^{C})C(O)—, —N(R^{C})C(O)(C₁-C₆-alkylene)-, —N(R^{C})C(O)(C₂-C₆-alkenylene)-, or —N(R^{C})—. In some embodiments, A is alkyl, alkenyl, alkynyl, heteroalkyl, cycloalkyl, heterocyclyl, aryl, heteroaryl, —O—, —C(O)O—, —C(O)—, —OC(O)—, or —N(R^{C})—. In some embodiments, A is alkyl, alkenyl, alkynyl, heteroalkyl, —O—, —C(O)O—, —C(O)—, —OC(O)—, or —N(R^{C})—. In some embodiments, A is alkyl, —O—, —C(O)O—, —C(O)—, —OC(O), or —N(R^{C})—. In some embodiments, A is —N(R^{C})C(O)—, —N(R^{C})C(O)(C₁-C₆-alkylene)-, or —N(R^{C})C(O)(C₁-C₆-alkenylene)-. In some embodiments, A is —N(R^{C})—. In some embodiments, A is —N(R^{C})—, and R^{C} an R^{D} is independently hydrogen or alkyl. In some embodiments, A is —NH—. In some embodiments, A is —N(R^{C})C(O)(C₁-C₆-alkylene)-, wherein alkylene is substituted with R¹. In some embodiments, A is —N(R^{C})C(O)(C₁-C₆-alkylene)-, and R¹ is alkyl (e.g., methyl). In some embodiments, A is —NHC(O)C(CH₃)₂—. In some embodiments, A is —N(R^{C})C(O)(methylene)-, and R¹ is alkyl (e.g., methyl). In some embodiments, A is —NHC(O)CH(CH₃)—. In some embodiments, A is —NHC(O)C(CH₃)—.

In some embodiments, for Formulas (I) or (I-a), L¹ is a bond, alkyl, or heteroalkyl. In some embodiments, L¹ is a bond or alkyl. In some embodiments, L¹ is a bond. In some embodiments, L¹ is alkyl. In some embodiments, L¹ is C₁-C₆ alkyl. In some embodiments, L¹ is —CH₂—, —CH(CH₃)—, —CH₂CH₂CH₂, or —CH₂CH₂—. In some embodiments, L¹ is —CH₂— or —CH₂CH₂—.

In some embodiments, for Formulas (I) or (I-a), L³ is a bond, alkyl, or heteroalkyl. In some embodiments, L³ is a bond. In some embodiments, L³ is alkyl. In some embodiments, L³ is C₁-C₁₂ alkyl. In some embodiments, L³ is C₁-C₆ alkyl. In some embodiments, L³ is —CH₂—. In some embodiments, L³ is heteroalkyl. In some embodiments, L³ is C₁-C₁₂ heteroalkyl, optionally substituted with one or more R² (e.g., oxo). In some embodiments, L³ is C₁-C₆ heteroalkyl, optionally substituted with one or more R² (e.g., oxo). In some embodiments, L³ is —C(O)OCH₂—, —CH₂(OCH₂CH₂)₂—, —CH₂(OCH₂CH₂)₃—, CH₂CH₂O—, or —CH₂O—. In some embodiments, L³ is —CH₂O—.

In some embodiments, for Formulas (I) or (I-a), M is absent, alkyl, heteroalkyl, aryl, or heteroaryl. In some embodiments, M is heteroalkyl, aryl, or heteroaryl. In some embodiments, M is absent. In some embodiments, M is alkyl (e.g., C₁-C₆ alkyl). In some embodiments, M is —CH₂—. In some embodiments, M is heteroalkyl (e.g., C₁-C₆ heteroalkyl). In some embodiments, M is (—OCH₂CH₂-)z, wherein z is an integer selected from 1 to 10. In some embodiments, z is an integer selected from 1 to 5. In some embodiments, M is —OCH₂CH₂—, (—OCH₂CH₂—)₂, (—OCH₂CH₂—)₃, (—OCH₂CH₂—)₄, or (—OCH₂CH₂—)₅. In some embodiments, M is —OCH₂CH₂—, (—OCH₂CH₂—)₂, (—OCH₂CH₂—)₃, or (—OCH₂CH₂—)₄. In some embodiments, M is (—OCH₂CH₂—)₃. In some embodiments, M is aryl. In some embodiments, M is phenyl. In some embodiments, M is unsubstituted phenyl. In some embodiments, M is

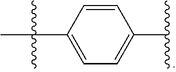 [SMILES: *c1ccc(*)cc1]

In some embodiments, M is phenyl substituted with R⁷ (e.g., 1 R⁷). In some embodiments, M is

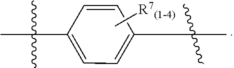

In some embodiments, R⁷ is CF₃.

In some embodiments, for Formulas (I) or (I-a), P is absent, heterocyclyl, or heteroaryl. In some embodiments, P is absent. In some embodiments, for Formulas (I) and (I-a), P is a tricyclic, bicyclic, or monocyclic heteroaryl. In some embodiments, P is a monocyclic heteroaryl. In some embodiments, P is a nitrogen-containing heteroaryl. In some embodiments, P is a monocyclic, nitrogen-containing heteroaryl. In some embodiments, P is a 5-membered heteroaryl. In some embodiments, P is a 5-membered nitrogen-containing heteroaryl. In some embodiments, P is tetrazolyl, imidazolyl, pyrazolyl, or triazolyl, pyrrolyl, oxazolyl, or thiazolyl. In some embodiments, P is tetrazolyl, imidazolyl, pyrazolyl, or triazolyl, or pyrrolyl. In some embodiments, P is imidazolyl. In some embodiments, P is

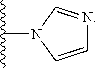 [SMILES: *n1ccnc1]

In some embodiments, P is triazolyl. In some embodiments, P is 1,2,3-triazolyl. In some embodiments, P is

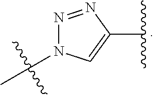 [SMILES: *c1cn(*)nn1]

In some embodiments, P is heterocyclyl. In some embodiments, P is a 5-membered heterocyclyl or a 6-membered heterocyclyl. In some embodiments, P is imidazolidinonyl. In some embodiments, P is

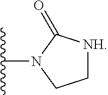 [SMILES: *N1CCNC1=O]

In some embodiments, P is thiomorpholinyl-1,1-dioxidyl.

In some embodiments, P is

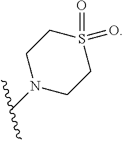 [SMILES: *N1CCS(=O)(=O)CC1]

In some embodiments, P is triazolyl substituted by one or more R⁴. In some embodiments, R⁴ is deuterium, alkyl, alkenyl, alkynyl, heteroalkyl, halogen, cyano, azido, —N(R^{C1})(R^{D1}), —N(R^{C1})C(O)R^{B1}, —C(O)N(R^{C1}), —S(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷. In some embodiments, R⁴ is deuterium, alkyl, heteroalkyl, halogen, cyano, or azido, wherein each alkyl and heteroalkyl is optionally substituted by one or more R⁷ (e.g., halogen). In some embodiments, P is

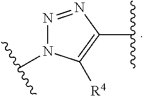

In some embodiments, P is triazolyl substituted by R⁴ (e.g., halogen). In some embodiments, R⁴ is deuterium, alkyl or halogen. In some embodiments, R⁴ is halogen (e.g., fluoro, chloro, bromo). In some embodiments, R⁴ is alkyl (e.g., —CH₃, —CH₂CH₃, —CF₃, —CH₂F, —CHF₂). In some embodiments, R⁴ is chloro. In some embodiments, P is

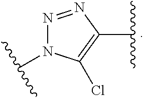 [SMILES: *c1nnn(*)c1Cl]

In some embodiments, P is

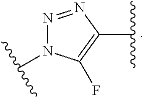 [SMILES: *c1nnn(*)c1F]

In some embodiments, P is

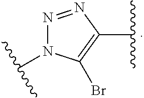 [SMILES: *c1nnn(*)c1Br]

In some embodiments, P is

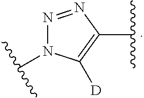 [SMILES: *c1nnn(*)c1[2H]]

In some embodiments, P is

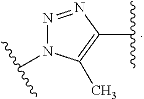 [SMILES: *c1nnn(*)c1C]

In some embodiments, P is

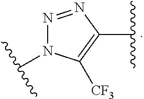 [SMILES: *c1nnn(*)c1C(F)(F)F]

In some embodiments, P is

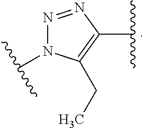 [SMILES: *c1nnn(*)c1CC]

In some embodiments, P is

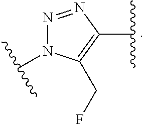 [SMILES: *c1nnn(*)c1CF]

In some embodiments, P is

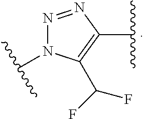 [SMILES: *c1nnn(*)c1C(F)F]

In some embodiments, for Formulas (I) or (I-a), Z is alkyl, heteroalkyl, cycloalkyl, heterocyclyl, aryl, or heteroaryl. In some embodiments, Z is heterocyclyl. In some embodiments, Z is monocyclic or bicyclic heterocyclyl. In some embodiments, Z is an oxygen-containing heterocyclyl. In some embodiments, Z is a 4-membered heterocyclyl, 5-membered heterocyclyl, or 6-membered heterocyclyl. In some embodiments, Z is a 6-membered heterocyclyl. In some embodiments, Z is a 6-membered oxygen-containing heterocyclyl. In some embodiments, Z is tetrahydropyranyl. In some embodiments, Z is

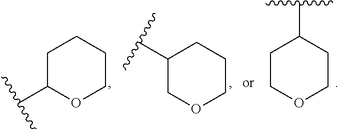

In some embodiments, Z is a 4-membered oxygen-containing heterocyclyl. In some embodiments, Z is

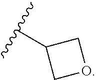 [SMILES: *C1COC1]

In some embodiments, Z is a bicyclic oxygen-containing heterocyclyl. In some embodiments, Z is phthalic anhydridyl. In some embodiments, Z is a sulfur-containing heterocyclyl. In some embodiments, Z is a 6-membered sulfur-containing heterocyclyl. In some embodiments, Z is a 6-membered heterocyclyl containing a nitrogen atom and a sulfur atom. In some embodiments, Z is thiomorpholinyl-1,1-dioxidyl. In some embodiments, Z is

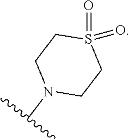 [SMILES: *N1CCS(=O)(=O)CC1]

In some embodiments, Z is a nitrogen-containing heterocyclyl. In some embodiments, Z is a 6-membered nitrogen-containing heterocyclyl. In some embodiments, Z is

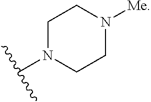 [SMILES: *N1CCN(C)CC1]

In some embodiments, Z is a bicyclic heterocyclyl. In some embodiments, Z is a bicyclic nitrogen-containing heterocyclyl, optionally substituted with one or more R⁵. In some embodiments, Z is 2-oxa-7-azaspiro[3.5]nonanyl. In some embodiments, Z is

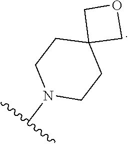 [SMILES: *N1CCC2(CC1)COC2]

In some embodiments, Z is 1-oxa-3,8-diazaspiro[4.5]decan-2-one. In some embodiments, Z is

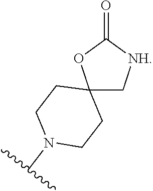 [SMILES: *N1CCC2(CC1)CNC(=O)O2]

In some embodiments, for Formulas (I) or (I-a), Z is aryl. In some embodiments, Z is monocyclic aryl. In some embodiments, Z is phenyl. In some embodiments, Z is monosubstituted phenyl (e.g., with 1 R⁵). In some embodiments, Z is monosubstituted phenyl, wherein the 1 R⁵ is a nitrogen-containing group. In some embodiments, Z is monosubstituted phenyl, wherein the 1 R⁵ is NH₂. In some embodiments, Z is monosubstituted phenyl, wherein the 1 R⁵ is an oxygen-containing group. In some embodiments, Z is monosubstituted phenyl, wherein the 1 R⁵ is an oxygen-containing heteroalkyl. In some embodiments, Z is monosubstituted phenyl, wherein the 1 R⁵ is OCH₃. In some embodiments, Z is monosubstituted phenyl, wherein the 1 R⁵ is in the ortho position. In some embodiments, Z is monosubstituted phenyl, wherein the 1 R⁵ is in the meta position. In some embodiments, Z is monosubstituted phenyl, wherein the 1 R⁵ is in the para position.

In some embodiments, for Formulas (I) or (I-a), Z is alkyl. In some embodiments, Z is C₁-C₁₂ alkyl. In some embodiments, Z is C₁-C₁₀ alkyl. In some embodiments, Z is C₁-C₈ alkyl. In some embodiments, Z is C₁-C₈ alkyl substituted with 1-5 R⁵. In some embodiments, Z is C₁-C₈ alkyl substituted with 1 R⁵. In some embodiments, Z is C₁-C₈ alkyl substituted with 1 R⁵, wherein R⁵ is alkyl, heteroalkyl, halogen, oxo, —OR^{A1}, —C(O)OR^{A1}, —C(O)R^{B1}, —OC(O)R^{B1}, or —N(R^{C1})(R^{D1}). In some embodiments, Z is C₁-C₈ alkyl substituted with 1 R⁵, wherein R⁵ is —OR^{A1} or —C(O)OR^{A1}. In some embodiments, Z is C₁-C₈ alkyl substituted with 1 R⁵, wherein R⁵ is —OR^{A1} or —C(O)OH. In some embodiments, Z is —CH₃.

In some embodiments, for Formulas (I) or (I-a), Z is heteroalkyl. In some embodiments, Z is C₁-C₁₂ heteroalkyl. In some embodiments, Z is C₁-C₁₀ heteroalkyl. In some embodiments, Z is C₁-C₈ heteroalkyl. In some embodiments, Z is C₁-C₆ heteroalkyl. In some embodiments, Z is a nitrogen-containing heteroalkyl optionally substituted with one or more R⁵. In some embodiments, Z is a nitrogen and sulfur-containing heteroalkyl substituted with 1-5 R⁵. In some embodiments, Z is N-methyl-2-(methylsulfonyl)ethan-1-aminyl.

In some embodiments, Z is —OR^{A} or —C(O)OR^{A}. In some embodiments, Z is —OR^{A} (e.g., —OH or —OCH₃). In some embodiments, Z is —OCH₃. In some embodiments, Z is —C(O)OR^{A} (e.g., —C(O)OH).

In some embodiments, Z is hydrogen.

In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound of Formula (I) is a compound of Formula (I-b):

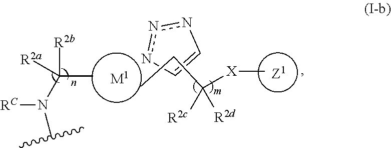

or a salt thereof, wherein Ring M¹ is cycloalkyl, heterocyclyl, aryl, or heteroaryl, each of which is optionally substituted with 1-5 R³; Ring Z¹ is cycloalkyl, heterocyclyl, aryl or heteroaryl, optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl, or each of R^{2a} and R^{2b} or R^{2c} and R^{2d} is taken together to form an oxo group; X is absent, N(R¹⁰), O, or S; R^{C} is hydrogen, alkyl, alkenyl, alkynyl, heteroalkyl, cycloalkyl, heterocyclyl, aryl, or heteroaryl, wherein each of alkyl, alkenyl, alkynyl, heteroalkyl, cycloalkyl, heterocyclyl, aryl, or heteroaryl is optionally substituted with 1-6 R⁶; each R³, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; R¹⁰ is hydrogen, alkyl, alkenyl, alkynyl, heteroalkyl, —C(O)OR^{A1}, —C(O)R^{B1}, —OC(O)R^{B1}, —C(O)N(R^{C1}), cycloalkyl, heterocyclyl, aryl, or heteroaryl; each R^{A1}, R^{B1}, R^{C1}, R^{D1} and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; each m and n is independently 1, 2, 3, 4, 5, or 6; and “ [symbol] ” refers to a connection to an attachment group or a polymer described herein. In some embodiments, for each R³ and R⁵, each alkyl, alkenyl, alkynyl, heteroalkyl, cycloalkyl, heterocyclyl, aryl, or heteroaryl is optionally and independently substituted with halogen, oxo, cyano, cycloalkyl, or heterocyclyl.

In some embodiments, the compound of Formula (I-b) is a compound of Formula (I-b-i):

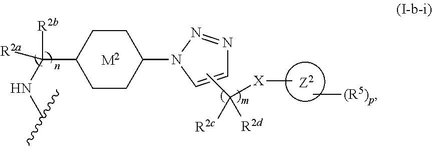

or a pharmaceutically acceptable sat thereof, wherein Ring M² is aryl or heteroaryl optionally substituted with one or more R³; Ring Z² is cycloalkyl, heterocyclyl, aryl, or heteroaryl; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; X is absent, O, or S; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; or two R⁵ are taken together to form a 5-6 membered ring fused to Ring Z²; each R^{A1} and R^{B1} is independently hydrogen, alkyl, or heteroalkyl; m and n are each independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I-b-i) is a compound of Formula (I-b-ii):

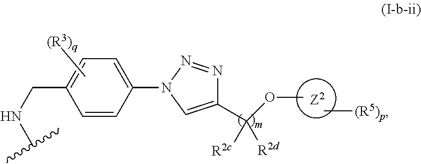

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R^{2d} and taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of p and q is independently 0, 1, 2, 3, 4, 5, or 6; m is 1, 2, 3, 4, 5, or 6; and “ [symbol] ” refers to a connection to an attachment group or a polymer described herein.

In some embodiments, the compound of Formula (I) is a compound of Formula (I-c):

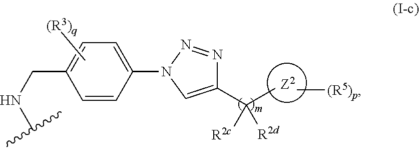

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R^{2d} is taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; m is 1, 2, 3, 4, 5, or 6; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-d):

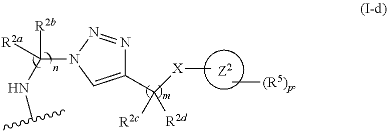

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-e):

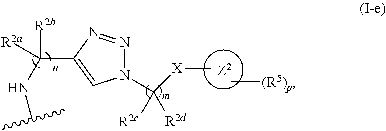

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-f):

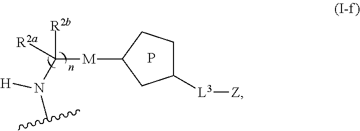

or a pharmaceutically acceptable salt thereof, wherein M is alkyl optionally substituted with one or more R³; Ring P is heteroaryl optionally substituted with one or more R⁴; L³ is alkyl or heteroalkyl optionally substituted with one or more R²; Z is alkyl, heteroalkyl, cycloalkyl, heterocyclyl, aryl, or heteroaryl, each of which is optionally substituted with one or more R⁵; each of R^{2a} and R^{2b} is independently hydrogen, alkyl, or heteroalkyl, or R^{2a} and R^{2b} is taken together to form an oxo group; each R², R³, R⁴, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (II):

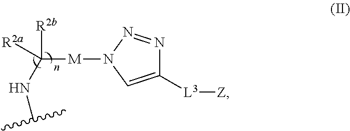

or a pharmaceutically acceptable salt thereof, wherein M is a bond, alkyl or aryl, wherein alkyl and aryl is optionally substituted with one or more R³; L³ is alkyl or heteroalkyl optionally substituted with one or more R²; Z is hydrogen, alkyl, heteroalkyl, cycloalkyl, heterocyclyl, aryl, heteroaryl or —OR^{A}, wherein alkyl, heteroalkyl, cycloalkyl, heterocyclyl, aryl, and heteroaryl is optionally substituted with one or more R⁵; R^{A} is hydrogen; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (II) is a compound of Formula (II-a):

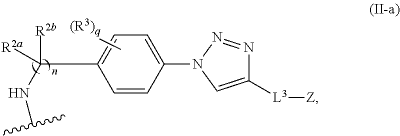

or a pharmaceutically acceptable salt thereof, wherein L³ is alkyl or heteroalkyl, each of which is optionally substituted with one or more R²; Z is hydrogen, alkyl, heteroalkyl, or —OR^{A}, wherein alkyl and heteroalkyl are optionally substituted with one or more R⁵; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, —C(O)OR^{A1}, or —C(O)R^{B1}; R^{A} is hydrogen; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (III):

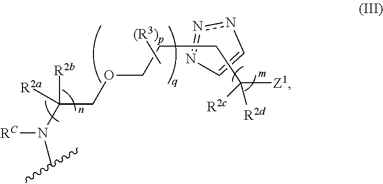

or a pharmaceutically acceptable salt thereof, wherein Z¹ is alkyl, alkenyl, alkynyl, heteroalkyl, cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl; or R^{2a} and R^{2b} or R^{2c} and R^{2d} are taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; q is an integer from 0 to 25; and “ [symbol] ” refers to a connection to an attachment group or a polymer described herein.

In some embodiments, the compound of Formula (III) is a compound of Formula (III-a):

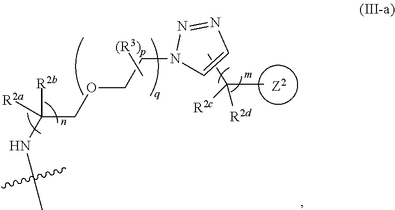

or a pharmaceutically acceptable salt thereof, wherein Ring Z² is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; and “ [symbol] ” refers to a connection to an attachment group or a polymer described herein.

In some embodiments, the compound of Formula (III-a) is a compound of Formula (III-b):

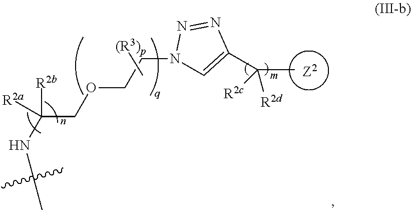

or a pharmaceutically acceptable salt thereof, wherein Ring Z² is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; and “ [symbol] ” refers to a connection to an attachment group or a polymer described herein.

In some embodiments, the compound of Formula (III-a) is a compound of Formula (III-c):

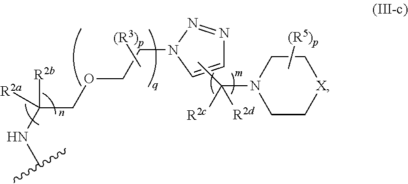

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4, or 5; q is an integer from 0 to 25; x is 0, 1, or 2; and “ [symbol] ” refers to a connection to an attachment group or a polymer described herein.

In some embodiments, the compound of Formula (III-c) is a compound of Formula (III-d):

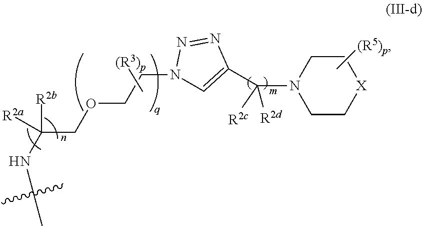

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4, or 5; q is an integer from 0 to 25; x is 0, 1, or 2; and “ [symbol] ” refers to a connection to an attachment group or a polymer described herein.

In some embodiments, the compound of Formula (I) is a compound of Formula (IV-a):

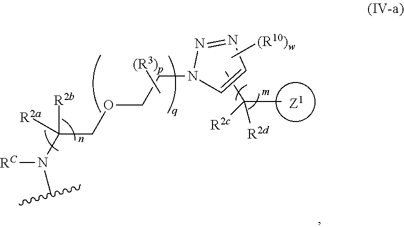

or a pharmaceutically acceptable salt thereof, wherein Ring Z¹ is heterocyclyl optionally substituted with 1-5 R⁵; R^{C} is hydrogen, alkyl, alkenyl, —C(O)(C₁-C₆-alkyl), or —C(O)(C₁-C₆-alkenyl), wherein each alkyl and alkenyl is optionally substituted with 1-6 R⁶; each of R^{2a}, R^{2b}, R^{2c}, and R^{2d} is independently hydrogen, alkyl, heteroalkyl, halo, or amino; or R^{2a} and R^{2b} or R^{2c} and R^{2d} are taken together to form an oxo group; each of R³, R⁵ and R⁶ is independently alkyl, heteroalkyl, halogen, oxo, —OR^{A1}, —C(O)OR^{A1}, —C(O)R^{B1}, —SR^{E1}, —S(O)ₓR^{E1}, or —OS(O)ₓR^{E1}; each R¹⁰ is independently deuterium, alkyl, haloalkyl, heteroalkyl, halo, cyano, nitro, or amino; each R^{A1}, R^{B1}, and R^{E1} is independently hydrogen, alkyl, or heteroalkyl; m and n are each independently 1, 2, 3, 4, 5, or 6; w is 0, 1, or 2; each of q and p is independently an integer from 0 to 25; and x is 0, 1, or 2.

In some embodiments, Ring Z¹ is heterocyclyl. In some embodiments, Ring Z¹ is nitrogen-containing heterocyclyl. In some embodiments, Ring Z¹ is 4-membered heterocyclyl or 6-membered heterocyclyl. In some embodiments, Ring Z¹ is heterocyclyl substituted with 1 R⁵. In some embodiments, R⁵ is —S(O)ₓR^{E1}. In some embodiments, R^{E1} is alkyl (e.g., —CH₃). In some embodiments, x is 2. In some embodiments, R⁵ is —S(O)₂(CH₃).

In some embodiments, each of R^{2a}, R^{2b}, R^{2c}, and R^{2d} is independently hydrogen.

In some embodiments, R^{C} is hydrogen, —C(O)(C₁-C₆-alkyl), or —C(O)(C₁-C₆-alkenyl). In some embodiments, R^{C} is hydrogen. In some embodiments, n is 1. In some embodiments, q is 2, 3, 4, or 5. In some embodiments, q is 3. In some embodiments, m is 1. In some embodiments, p is 0. In some embodiments, w is 0. In some embodiments, w is 1. In some embodiments, R¹⁰ is halo (e.g., Cl).

In some embodiments, the compound of Formula (I) is a compound of Formula (IV-b):

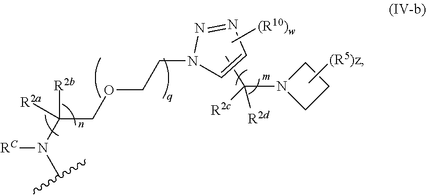

or a pharmaceutically acceptable salt thereof, wherein R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R⁵ and R⁶ is independently alkyl, heteroalkyl, halogen, oxo, —S(O)ₓR^{E1}, or —OS(O)ₓR^{E1}; each R¹⁰ is independently deuterium, alkyl, haloalkyl, heteroalkyl, halo, cyano, nitro, or amino; R^{E1} is independently hydrogen, alkyl, or heteroalkyl; m and n are each independently 1, 2, 3, 4, 5, or 6; w is 0, 1, or 2; q is an integer from 0 to 25; x is 0, 1, or 2; and z is 0, 1, 2, 3, 4, 5, or 6.

In some embodiments, R⁵ is —S(O)ₓR^{E1}. In some embodiments, R^{E1} is alkyl (e.g., —CH₃). In some embodiments, x is 2. In some embodiments, R⁵ is —S(O)₂(CH₃). In some embodiments, z is 1. In some embodiments, each of R^{2a}, R^{2b}, R^{2c}, and R^{2d} is independently hydrogen.

In some embodiments, R^{C} is hydrogen, —C(O)(C₁-C₆-alkyl), or —C(O)(C₁-C₆-alkenyl). In some embodiments, R^{C} is hydrogen.

In some embodiments, n is 1. In some embodiments, q is 2, 3, 4, or 5. In some embodiments, q is 3. In some embodiments, m is 1. In some embodiments, w is 0.

In some embodiments, the compound of Formula (I) is a compound of Formula (IV-c):

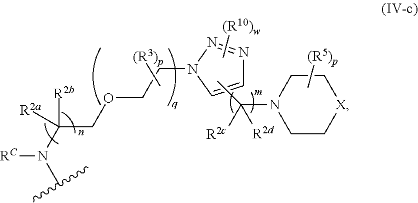

or a pharmaceutically acceptable salt thereof, wherein X is C(R′)(R″), N(R′), or S(O)ₓ; each of R′ and R″ is independently hydrogen, alkyl, or halogen; each of R^{2a}, R^{2b}, R^{2c}, and R^{2d} is independently hydrogen, alkyl, heteroalkyl, or halogen; or R^{2a} and R^{2b} or R^{2c} and R^{2d} are taken together to form an oxo group; R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R³, R⁵, and R⁶ is independently alkyl, heteroalkyl, halogen, oxo, —OR^{A1}, —C(O)OR^{A1}, —C(O)R^{B}, —SR^{E1}, —S(O)ₓR^{E1}, or —OS(O)ₓR^{E1}; each R¹⁰ is independently deuterium, alkyl, haloalkyl, heteroalkyl, halo, cyano, nitro, or amino; each R^{A1}, R^{B1}, and R^{E1} is independently hydrogen, alkyl, or heteroalkyl; m and n are each independently 1, 2, 3, 4, 5, or 6; w is 1; each of q and p is independently an integer from 0 to 25; and x is 0, 1, or 2.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, R^{C} is independently, —C(O)(C₁-C₆-alkyl), or —C(O)(C₁-C₆-alkenyl).

In some embodiments, R^{C} is hydrogen.

In some embodiments, n is 1. In some embodiments, q is 2, 3, 4, or 5. In some embodiments, q is 3. In some embodiments, m is 1. In some embodiments, p is 0. In some embodiments, R¹⁰ is halo (e.g., Cl).

In some embodiments, the compound of Formula (I) is a compound of Formula (IV-d):

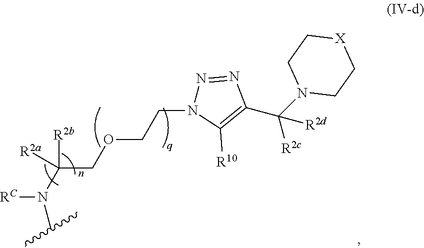

or a pharmaceutically acceptable salt thereof, wherein X is C(R′)(R″), N(R′), or S(O)ₓ; each of R′ and R″ is independently hydrogen, alkyl, or halogen; each of R^{2a}, R^{2b}, R^{2c}, and R^{2d} is independently hydrogen, alkyl, heteroalkyl, or halogen; or R^{2a} and R^{2b} or R^{2c} and R^{2d} are taken together to form an oxo group; R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R⁶ is independently alkyl, heteroalkyl, halogen, oxo, —OR^{A1}, —C(O)OR^{A1}, —C(O)R^{B1}; each R¹⁰ is independently deuterium, alkyl, haloalkyl, heteroalkyl, halo, cyano, nitro, or amino; each R^{A1} and R^{B1} is independently hydrogen, alkyl, or heteroalkyl; n is 1, 2, 3, 4, 5, or 6; q is an integer from 0 to 25; and x is 0, 1, or 2.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, R^{C} is hydrogen, —C(O)(C₁-C₆-alkyl), or —C(O)(C₁-C₆-alkenyl). In some embodiments, R^{C} is hydrogen.

In some embodiments, n is 1. In some embodiments, q is 2, 3, 4, or 5. In some embodiments, q is 3. In some embodiments, m is 1. In some embodiments, p is 0. In some embodiments, R¹⁰ is halo (e.g., Cl).

In some embodiments, the compound of Formula (I) is a compound of Formula (IV-e):

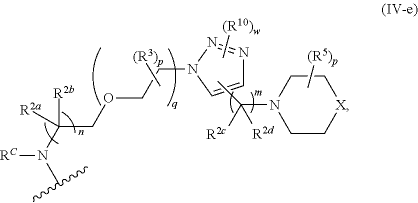

or a pharmaceutically acceptable salt thereof, wherein X is C(R′)(R″), N(R′), or S(O)ₓ; each of R′ and R″ is independently hydrogen, alkyl, or halogen; each of R^{2a}, R^{2b}, R^{2c}, and R^{2d} is independently hydrogen, alkyl, heteroalkyl, or halogen; or R^{2a} and R^{2b} or R^{2c} and R^{2d} are taken together to form an oxo group; R^{C} is independently hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R³, R⁵, and R⁶ is independently alkyl, heteroalkyl, halogen, oxo, —OR^{A1}, —C(O)OR^{A1}, —C(O)R^{B1}, —SR^{E1}, —S(O)ₓR^{E1}, or —OS(O)ₓR^{E1}; each R¹⁰ is independently deuterium, alkyl, haloalkyl, heteroalkyl, halo, cyano, nitro, or amino, wherein each alkyl or heteroalkyl is optionally substituted by one or more R¹¹; each R¹¹ is independently alkyl, alkenyl, alkynyl, heteroalkyl, halogen, cyano, oxo, hydroxyl, cycloalkyl, or heterocyclyl; each R^{A1}, R^{B1}, and R^{E1} is independently hydrogen, alkyl, or heteroalkyl; m and n are each independently 1, 2, 3, 4, 5, or 6; w is 1; each of q and p is independently an integer from 0 to 25; and x is 0, 1, or 2.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, R¹⁰ is deuterium, alkyl, heteroalkyl, halogen, cyano, or azido, wherein each alkyl and heteroalkyl is optionally substituted by one or more R¹¹ (e.g., halogen). In some embodiments, R¹⁰ is deuterium, alkyl, or halogen. In some embodiments, R¹⁰ is halogen (e.g., fluoro, chloro, bromo). In some embodiments, R¹⁰ is alkyl (e.g., —CH₃, —CH₂CH₃, —CF₃, —CH₂F, —CHF₂).

In some embodiments, R^{C} is hydrogen, —C(O)(C₁-C₆-alkyl), or —C(O)(C₁-C₆-alkenyl). In some embodiments, R^{C} is hydrogen.

In some embodiments, n is 1. In some embodiments, q is 2, 3, 4, or 5. In some embodiments, q is 3. In some embodiments, m is 1. In some embodiments, p is 0.

In some embodiments, the compound is a compound of Formula (I). In some embodiments, L² is a bond and P and L³ are independently absent.

In some embodiments, the compound is a compound of Formula (I-a). In some embodiments of Formula (II-a), L² is a bond, P is heteroaryl, L³ is a bond, and Z is hydrogen. In some embodiments, P is heteroaryl, L³ is heteroalkyl, and Z is alkyl. In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound is a compound of Formula (I-b). In some embodiments, P is absent, L¹ is —NHCH₂, L² is a bond, M is aryl (e.g., phenyl), L³ is —CH₂O, and Z is heterocyclyl (e.g., a nitrogen-containing heterocyclyl, e.g., thiomorpholinyl-1,1-dioxide). In some embodiments, the compound of Formula (I-b) is Compound 116.

In some embodiments of Formula (I-b), P is absent, L¹ is —NHCH₂, L² is a bond, M is absent, L³ is a bond, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-b) is Compound 105.

In some embodiments, the compound is a compound of Formula (I-b-i). In some embodiments of Formula (I-b-i), each of R^{2a} and R^{2b} is independently hydrogen or CH₃, each of R^{2c} and R^{2d} is independently hydrogen, m is 1 or 2, n is 1, X is 0, p is 0, M² is phenyl optionally substituted with one or more R³, R³ is —CF₃, and Z² is heterocyclyl (e.g., an oxygen-containing heterocyclyl, e.g., tetrahydropyranyl, tetrahydrofuranyl, oxetanyl, or oxiranyl). In some embodiments, the compound of Formula (I-b-i) is Compound 100, Compound 106, Compound 107, Compound 108, Compound 109, or Compound 111.

In some embodiments, the compound is a compound of Formula (I-b-ii). In some embodiments of Formula (I-b-ii), each of R^{2a}, R^{2b}, R^{2c}, and R^{2d} is independently hydrogen, q is 0, p is 0, m is 1, and Z² is heterocyclyl (e.g., an oxygen-containing heterocyclyl, e.g., tetrahydropyranyl). In some embodiments, the compound of Formula (I-b-ii) is Compound 100.

In some embodiments, the compound is a compound of Formula (I-c). In some embodiments of Formula (I-c), each of R^{2c} and R^{2d} is independently hydrogen, m is 1, p is 1, q is 0, R⁵ is —CH₃, and Z is heterocyclyl (e.g., a nitrogen-containing heterocyclyl, e.g., piperazinyl). In some embodiments, the compound of Formula (I-c) is Compound 113.

In some embodiments, the compound is a compound of Formula (I-d). In some embodiments of Formula (I-d), each of R^{2a}, R^{2b}, R^{2c}, and R^{2d} is independently hydrogen, m is 1, n is 3, X is 0, p is 0, and Z is heterocyclyl (e.g., an oxygen-containing heterocyclyl, e.g., tetrahydropyranyl, tetrahydrofuranyl, oxetanyl, or oxiranyl). n some embodiments, the compound of Formula (I-d) is Compound 110 or Compound 114.

In some embodiments, the compound is a compound of Formula (I-f). In some embodiments of Formula (I-f), each of R^{2a} and R^{2b} is independently hydrogen, n is 1, M is —CH₂—, P is a nitrogen-containing heteroaryl (e.g., imidazolyl), L³ is —C(O)OCH₂—, and Z is CH₃. In some embodiments, the compound of Formula (I-f) is Compound 115.

In some embodiments, the compound is a compound of Formula (II-a). In some embodiments of Formula (II-a), each of R^{2a} and R^{2b} is independently hydrogen, n is 1, q is 0, L³ is —CH₂(OCH₂CH₂)₂, and Z is —OCH₃. In some embodiments, the compound of Formula (II-a) is Compound 112.

In some embodiments of Formula (II-a), each of R^{2a} and R^{2b} is independently hydrogen, n is 1, L³ is a bond or —CH₂, and Z is hydrogen or —OH. In some embodiments, the compound of Formula (II-a) is Compound 103 or Compound 104.

In some embodiments, the compound is a compound of Formula (III). In some embodiments of Formula (III), each of R^{2a}, R^{2b}, R^{2c}, and R^{2d} is independently hydrogen, m is 1, n is 2, q is 3, p is 0, R^{C} is hydrogen, and Z¹ is heteroalkyl optionally substituted with R⁵ (e.g., —N(CH₃)(CH₂CH₂)S(O)₂CH₃). In some embodiments, the compound of Formula (III) is Compound 120.

In some embodiments, the compound is a compound of Formula (III-b). In some embodiments of Formula (III-b), each of R^{2a}, R^{2b}, R^{2c}, and R^{2d} is independently hydrogen, m is 0, n is 2, q is 3, p is 0, and Z² is aryl (e.g., phenyl) substituted with 1 R⁵ (e.g., —NH₂). In some embodiments, the compound of Formula (III-b) is Compound 102.

In some embodiments, the compound is a compound of Formula (III-b). In some embodiments of Formula (III-b), each of R^{2a}, R^{2b}, R^{2c}, and R^{2d} is independently hydrogen, m is 1, n is 2, q is 3, p is 0, R^{C} is hydrogen, and Z² is heterocyclyl (e.g., a nitrogen-containing heterocyclyl, e.g., a nitrogen-containing spiro heterocyclyl, e.g., 2-oxa-7-azaspiro[3.5]nonanyl). In some embodiments, the compound of Formula (III-a) is Compound 121.

In some embodiments, the compound is a compound of Formula (III-d). In some embodiments of Formula (III-d), each of R^{2a}, R^{2b}, R^{2c}, and R^{2d} is independently hydrogen, m is 1, n is 2, q is 1, 2, 3, or 4, p is 0, and X is S(O)₂. In some embodiments of Formula (III-d), each of R^{2a} and R^{2b} is independently hydrogen, m is 1, n is 2, q is 1, 2, 3, or 4, p is 0, and X is S(O)₂. In some embodiments, the compound of Formula (III-d) is Compound 101, Compound 117, Compound 118, or Compound 119.

In some embodiments, the compound is a compound of Formula (I-b), (I-d), or (I-e). In some embodiments, the compound is a compound of Formula (I-b), (I-d), or (II). In some embodiments, the compound is a compound of Formula (I-b), (I-d), or (I-f). In some embodiments, the compound is a compound of Formula (I-b), (I-d), or (III).

In some embodiments, the compound is a compound of Formula (IV-a) or (IV-b). In some embodiments, the compound is a compound of Formula (IV-a), (IV-c), (IV-d), or (IV-e). In some embodiments, each of R^{2a}, R^{2b}, R^{2c}, and R^{2d} is independently hydrogen, m is 1, n is 1, q is 1, 2, 3, or 4, w is 1, and X is S(O)₂. In some embodiments, the compound is any one of Compounds 122-154. In some embodiments, the compound of Formula (I) comprises a deuterium (e.g., R⁴ or R¹⁰ is a deuterium). In some embodiments, the compound of Formula (I) does not comprise a deuterium (e.g., R⁴ or R¹⁰ is not a deuterium).

Exemplary compounds of Formula (I) may be prepared as described in WO 2019/169333, WO 2021/119522 or any other method known to those skilled in the art.

In some embodiments, the compound of Formula (I) is not a compound disclosed in WO 2012/112982, WO 2012/167223, WO 2014/153126, WO 2016/019391, WO 2017/075630, US 2012/0213708, US 2016/0030359 or US 2016/0030360.

In some embodiments, the compound of Formula (I) comprises a compound shown in Table 3, or a pharmaceutically acceptable salt thereof. In some embodiments, a device described herein comprises a compound shown in Table 4, or a pharmaceutically acceptable salt thereof.

TABLE 4
Exemplary compounds of Formula (I)

| Compound | |
|---|---|
| No. | Structure |
| 100 | 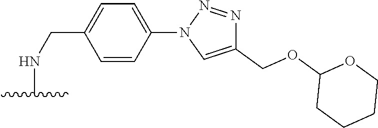 [SMILES: *NCc1ccc(-n2cc(COC3CCCCO3)nn2)cc1] |
| 101 | 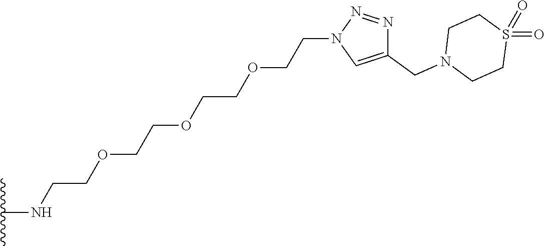 [SMILES: *NCCOCCOCCOCCn1cc(CN2CCS(=O)(=O)CC2)nn1] |
| 102 | 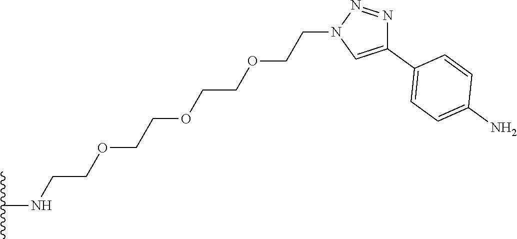 [SMILES: *NCCOCCOCCOCCn1cc(-c2ccc(N)cc2)nn1] |
| 103 | 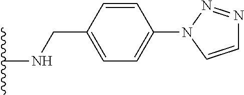 [SMILES: *NCc1ccc(-n2ccnn2)cc1] |
| 104 | 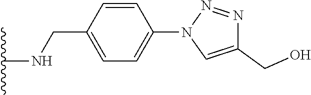 [SMILES: *NCc1ccc(-n2cc(CO)nn2)cc1] |
| 105 | 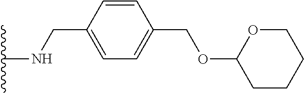 [SMILES: *NCc1ccc(COC2CCCCO2)cc1] |
| 106 | 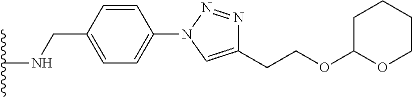 [SMILES: *NCc1ccc(-n2cc(CCOC3CCCCO3)nn2)cc1] |
| 107 | 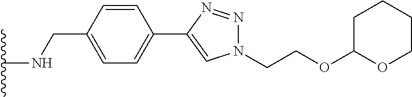 [SMILES: *NCc1ccc(-c2cn(CCOC3CCCCO3)nn2)cc1] |
| 108 | 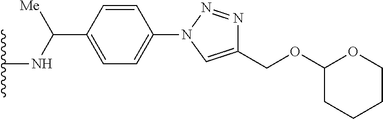 [SMILES: *NC(C)c1ccc(-n2cc(COC3CCCCO3)nn2)cc1] |
| 109 | 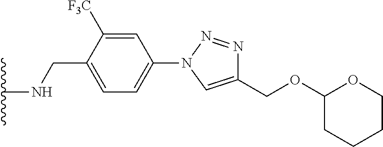 [SMILES: *NCc1ccc(-n2cc(COC3CCCCO3)nn2)cc1C(F)(F)F] |
| 110 | 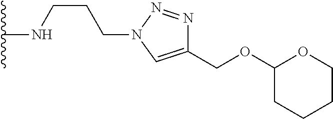 [SMILES: *NCCCn1cc(COC2CCCCO2)nn1] |
| 111 | 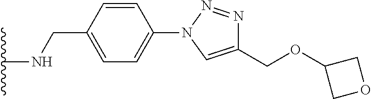 [SMILES: *NCc1ccc(-n2cc(COC3COC3)nn2)cc1] |
| 112 | 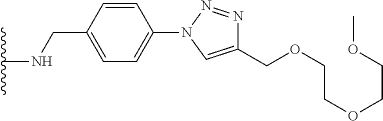 [SMILES: *NCc1ccc(-n2cc(COCCOCCOC)nn2)cc1] |
| 113 | 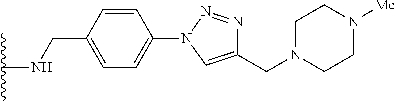 [SMILES: *NCc1ccc(-n2cc(CN3CCN(C)CC3)nn2)cc1] |
| 114 | 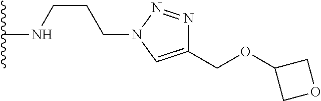 [SMILES: *NCCCn1cc(COC2COC2)nn1] |
| 115 | 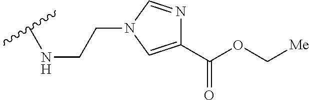 [SMILES: *NCCn1cnc(C(=O)OCC)c1] |
| 116 | 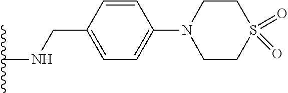 [SMILES: *NCc1ccc(N2CCS(=O)(=O)CC2)cc1] |
| 117 | 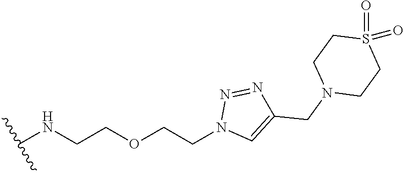 [SMILES: *NCCOCCn1cc(CN2CCS(=O)(=O)CC2)nn1] |
| 118 | 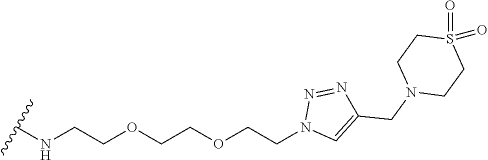 [SMILES: *NCCOCCOCCn1cc(CN2CCS(=O)(=O)CC2)nn1] |
| 119 | 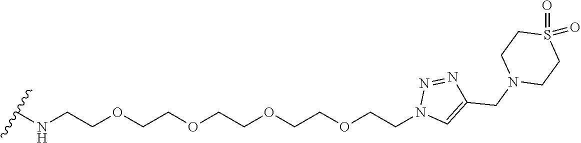 [SMILES: *NCCOCCOCCOCCOCCn1cc(CN2CCS(=O)(=O)CC2)nn1] |
| 120 | 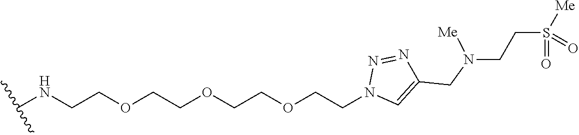 [SMILES: *NCCOCCOCCOCCn1cc(CN(C)CCS(C)(=O)=O)nn1] |
| 121 | 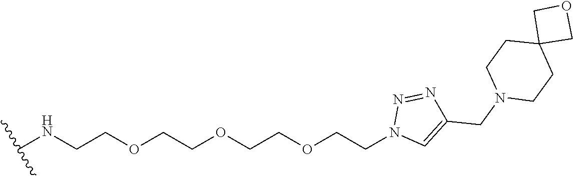 [SMILES: *NCCOCCOCCOCCn1cc(CN2CCC3(CC2)COC3)nn1] |
| 122 | 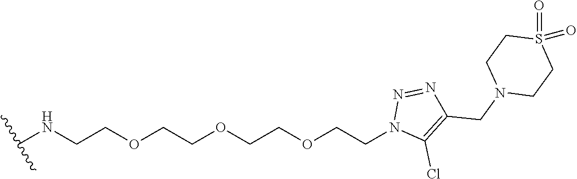 [SMILES: *NCCOCCOCCOCCn1nnc(CN2CCS(=O)(=O)CC2)c1Cl] |
| 123 | 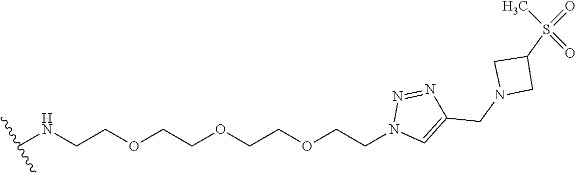 [SMILES: *NCCOCCOCCOCCn1cc(CN2CC(S(C)(=O)=O)C2)nn1] |
| 124 | 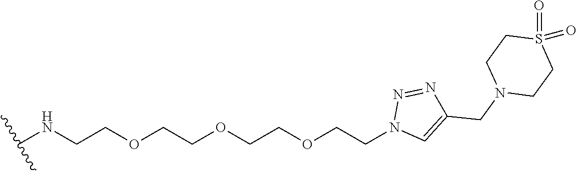 [SMILES: *NCCOCCOCCOCCn1cc(CN2CCS(=O)(=O)CC2)nn1] |
| 125 | 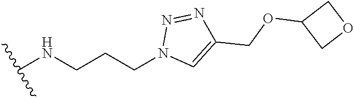 [SMILES: *NCCCn1cc(COC2COC2)nn1] |
| 126 | 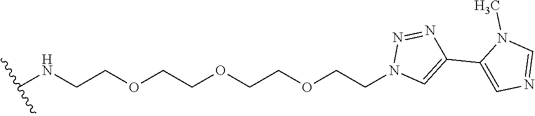 [SMILES: *NCCOCCOCCOCCn1cc(-c2cncn2C)nn1] |
| 127 | 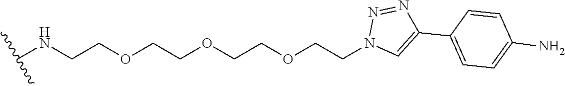 [SMILES: *NCCOCCOCCOCCn1cc(-c2ccc(N)cc2)nn1] |
| 128 | 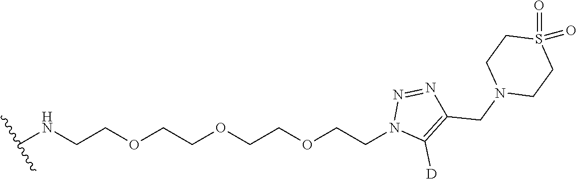 [SMILES: *NCCOCCOCCOCCn1nnc(CN2CCS(=O)(=O)CC2)c1[2H]] |
| 129 | 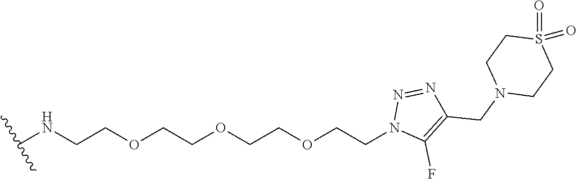 [SMILES: *NCCOCCOCCOCCn1nnc(CN2CCS(=O)(=O)CC2)c1F] |
| 130 | 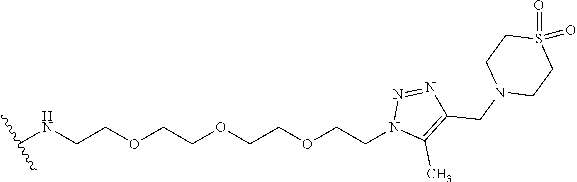 [SMILES: *NCCOCCOCCOCCn1nnc(CN2CCS(=O)(=O)CC2)c1C] |
| 131 | 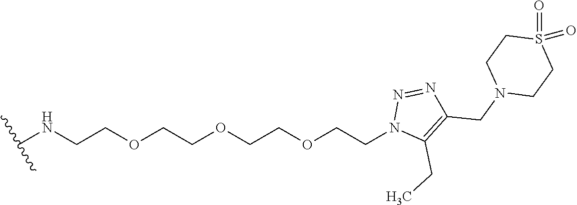 [SMILES: *NCCOCCOCCOCCn1nnc(CN2CCS(=O)(=O)CC2)c1CC] |
| 132 | 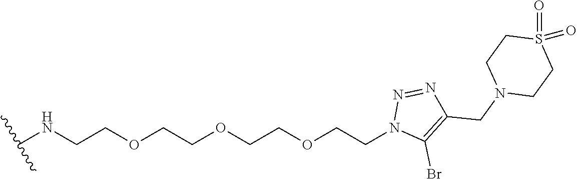 [SMILES: *NCCOCCOCCOCCn1nnc(CN2CCS(=O)(=O)CC2)c1Br] |
| 133 | 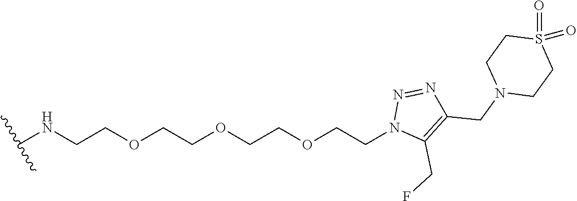 [SMILES: *NCCOCCOCCOCCn1nnc(CN2CCS(=O)(=O)CC2)c1CF] |
| 134 | 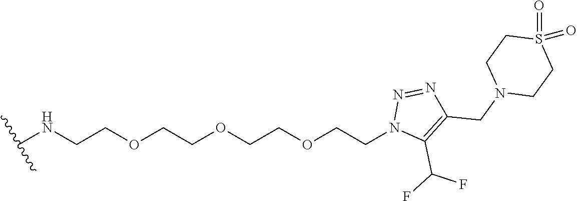 [SMILES: *NCCOCCOCCOCCn1nnc(CN2CCS(=O)(=O)CC2)c1C(F)F] |
| 135 | 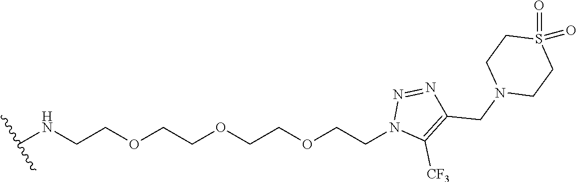 [SMILES: *NCCOCCOCCOCCn1nnc(CN2CCS(=O)(=O)CC2)c1C(F)(F)F] |
| 136 | 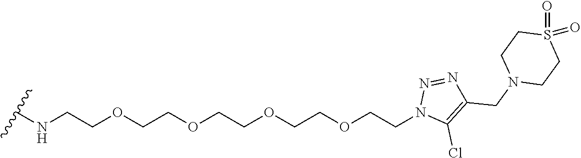 [SMILES: *NCCOCCOCCOCCOCCn1nnc(CN2CCS(=O)(=O)CC2)c1Cl] |
| 137 | 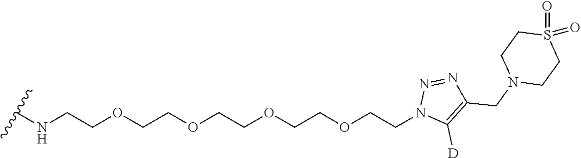 [SMILES: *NCCOCCOCCOCCOCCn1nnc(CN2CCS(=O)(=O)CC2)c1[2H]] |
| 138 | 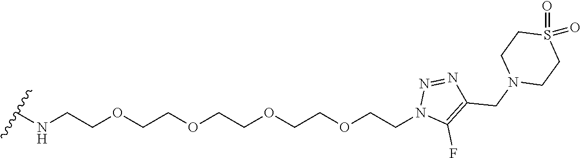 [SMILES: *NCCOCCOCCOCCOCCn1nnc(CN2CCS(=O)(=O)CC2)c1F] |
| 139 | 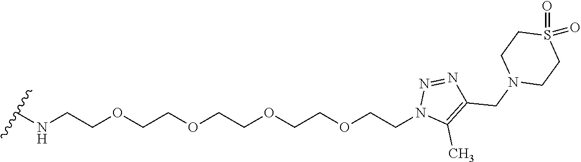 [SMILES: *NCCOCCOCCOCCOCCn1nnc(CN2CCS(=O)(=O)CC2)c1C] |
| 140 | 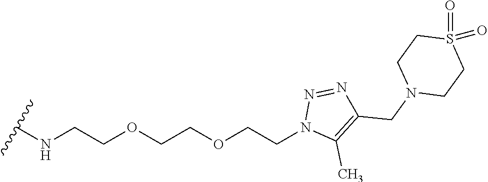 [SMILES: *NCCOCCOCCn1nnc(CN2CCS(=O)(=O)CC2)c1C] |
| 141 | 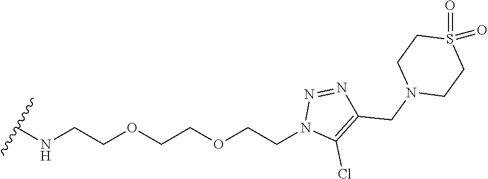 [SMILES: *NCCOCCOCCn1nnc(CN2CCS(=O)(=O)CC2)c1Cl] |
| 142 | 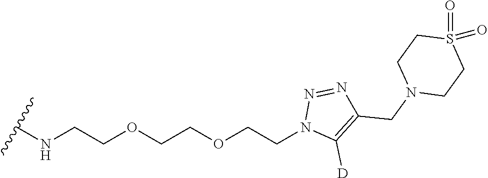 [SMILES: *NCCOCCOCCn1nnc(CN2CCS(=O)(=O)CC2)c1[2H]] |
| 143 | 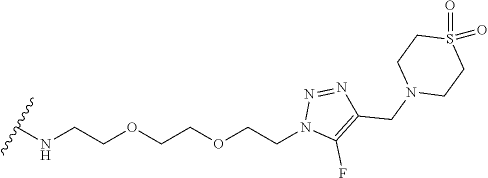 [SMILES: *NCCOCCOCCn1nnc(CN2CCS(=O)(=O)CC2)c1F] |
| 144 | 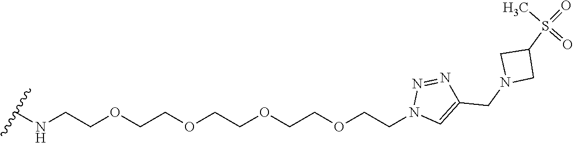 [SMILES: *NCCOCCOCCOCCOCCn1cc(CN2CC(S(C)(=O)=O)C2)nn1] |
| 145 | 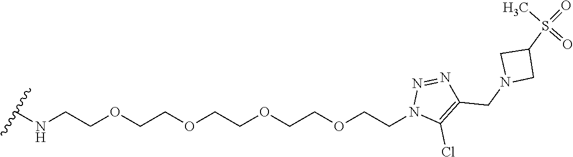 [SMILES: *NCCOCCOCCOCCOCCn1nnc(CN2CC(S(C)(=O)=O)C2)c1Cl] |
| 146 | 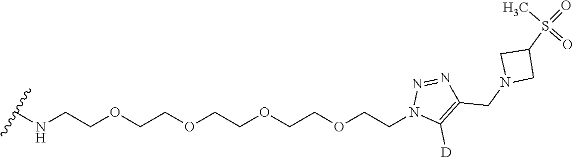 [SMILES: *NCCOCCOCCOCCOCCn1nnc(CN2CC(S(C)(=O)=O)C2)c1[2H]] |
| 147 | 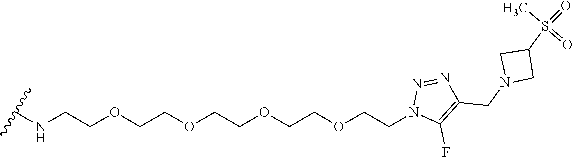 [SMILES: *NCCOCCOCCOCCOCCn1nnc(CN2CC(S(C)(=O)=O)C2)c1F] |
| 148 | 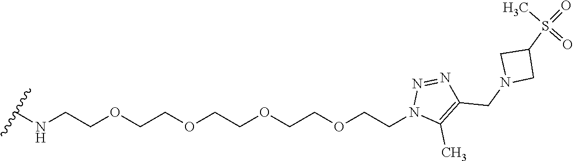 [SMILES: *NCCOCCOCCOCCOCCn1nnc(CN2CC(S(C)(=O)=O)C2)c1C] |
| 149 | 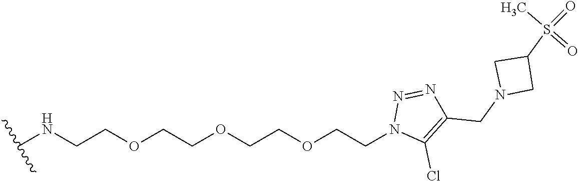 [SMILES: *NCCOCCOCCOCCn1nnc(CN2CC(S(C)(=O)=O)C2)c1Cl] |
| 150 | 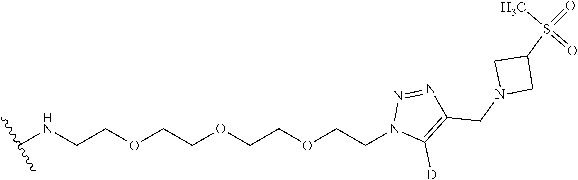 [SMILES: *NCCOCCOCCOCCn1nnc(CN2CC(S(C)(=O)=O)C2)c1[2H]] |
| 151 | 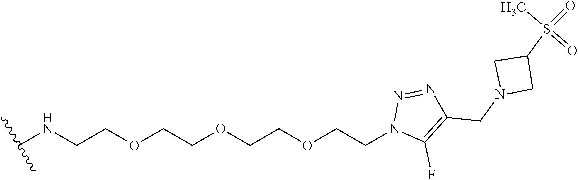 [SMILES: *NCCOCCOCCOCCn1nnc(CN2CC(S(C)(=O)=O)C2)c1F] |
| 152 | 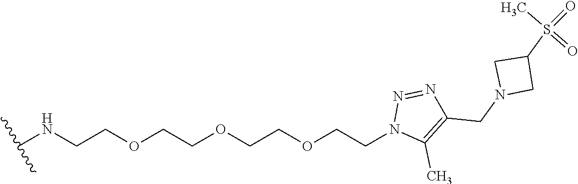 [SMILES: *NCCOCCOCCOCCn1nnc(CN2CC(S(C)(=O)=O)C2)c1C] |
| 153 | 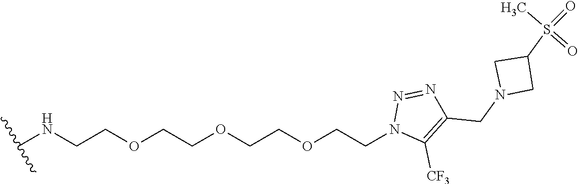 [SMILES: *NCCOCCOCCOCCn1nnc(CN2CC(S(C)(=O)=O)C2)c1C(F)(F)F] |
| 154 | 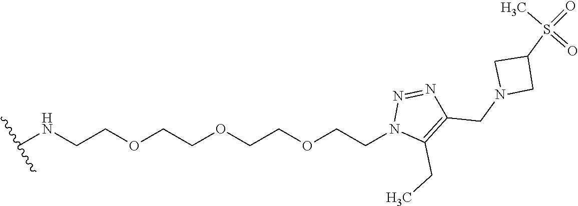 [SMILES: *NCCOCCOCCOCCn1nnc(CN2CC(S(C)(=O)=O)C2)c1CC] |

In some embodiments, the compound is a compound of Formula (I) (e.g., Formulas (I-a), (I-b), (I-b-i), (I-b-ii), (I-c), (I-d), (I-e), (I-f), (II), (II-a), (III), (III-a), (III-b), (III-c), (III-d), (IV-a), (IV-b), (IV-c), (IV-d), or (IV-e)), or a pharmaceutically acceptable salt thereof, and is selected from:

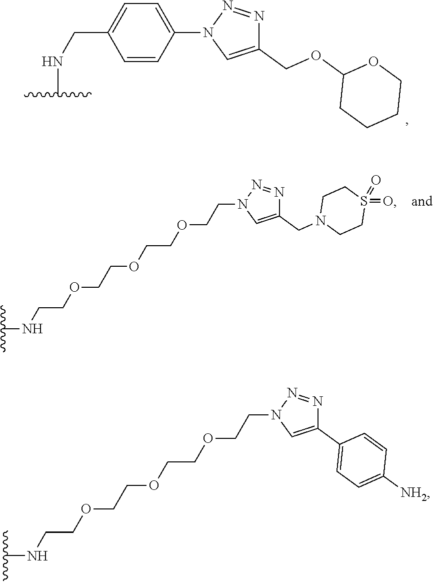

or a pharmaceutically acceptable salt thereof.

In some embodiments, the hydrogel capsules in a hydrogel capsule population described herein comprises the compound of

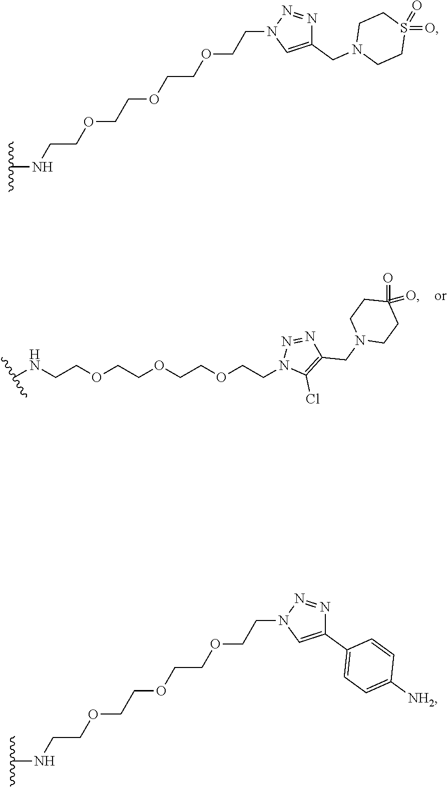

or a pharmaceutically acceptable salt of any one of these compounds.

In some embodiments, the hydrogel capsules in a hydrogel capsule population described herein comprises the compound of

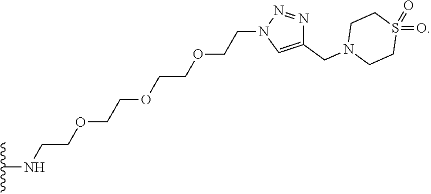 [SMILES: *NCCOCCOCCOCCn1cc(CN2CCS(=O)(=O)CC2)nn1]

In some embodiments, a compound of Formula (I) (e.g., Compound 101 in Table 4) is covalently attached to an alginate (e.g., an alginate with approximate MW<75 kDa, G:M ratio≥1.5) at a conjugation density of at least 2.0% and less than 9.0%, or 3.0% to 8.0%, 4.0-7.0, 5.0 to 7.0, or 6.0 to 7.0 or about 6.8 as determined by combustion analysis for percent nitrogen as described in WO 2020/069429. In an embodiment, the conjugation density of Compound 101 in the modified alginate is determined by quantitative free amine analysis, e.g., as described in WO2020198695, wherein the determined conjugation density is 1.0% w/w to 3.0% w/w, 1.3% w/w to 2.8% w/w, 1.3% w/w to 2.6% w/w, 1.5% w/w to 2.4% w/w, 1.5% w/w to 2.2% w/w, or 1.7% w/w to 2.2% w/w.

In an embodiment, the hydrogel capsules in a hydrogel capsule population described herein comprise a compound of Formula (I) (e.g., a compound shown in Table 4) covalently bound to an alginate polymer. The alginate polymer can be chemically modified with a compound of Formula (I) using any suitable method known in the art, e.g., as described in WO 2019/195055.

### Cells and Therapeutic Substances

The cells contained in hydrogel capsules described herein, e.g., in a population of hydrogel capsules, may be derived from a variety of different cell types (e.g., human cells), including epithelial cells, endothelial cells, fibroblast cells, islet cells (as defined herein), mesenchymal stem cells, induced pluripotent stem cells (iPSCs) and keratinocyte cells. Exemplary cell types include the cell types recited in WO 2017/075631. In an embodiment, the cells are not islet cells (as defined herein). In an embodiment, the cells are islet cells. In some embodiments, the cells are derived from a cell-line shown in Table 5 below.

TABLE 5
Exemplary cell lines

| | | Germ | |
|---|---|---|---|
| Cell Line | Cell Type | Layer | Commercial Source |
| ARPE-19 | Epithelial (Retinal) | Ectoderm | ATCC (CRL-2302) |
| BJ | Fibroblast (Foreskin) | Ectoderm | ATCC (CRL-2522) |
| CCD-841- | Epithelial (Colon) | Endoderm | ATCC (CRL-1790) |
| CoN | | | |
| HaCat | Keratinocyte | Ectoderm | Addexbio (T0020001) |
| HHSEC | Endothelial | Endoderm | Sciencellonline.com |
| | (Hepatic Sinusoidal) | | (#5000) |
| Huv-EC-C | Endothelial | Mesoderm | ATCC (CRL-1730) |
| | (Embryonic umbilical) | | |
| MCF-10A | Epithelial | Ectoderm | ATCC (CRL-10317) |
| | (Mammary Gland) | | |
| MRC-5 | Fibroblast (Lung) | Mesoderm | ATCC (CCL-171) |
| MSC, | Mesenchyme | Mesoderm | ATCC |
| human | (Bone Marrow) | | (PCS-500-012) |
| MSC, | Mesenchyme | Mesoderm | Cyagen |
| mouse | (Bone Marrow) | | (MU BMX-01001) |
| WS-1 | Fibroblast (Skin) | Ectoderm | ATCC (CRL-1502) |
| 293F | Epithelial | Mesoderm | Thermo Fisher |
| | (Embryonic Kidney) | | (R790007) |

Cells may be genetically modified to express and secrete a therapeutic substance of interest using any of a variety of genetic engineering techniques known in the art. For example, a cell may be transfected with an expression vector comprising a nucleotide sequence encoding a desired polypeptide operably linked to control elements necessary or useful for gene expression, e.g., promoter, ribosomal binding site, enhancer, polyA signal and the like.

In some embodiments, the expression vector encodes a polypeptide intended to provide a therapeutic effect when the hydrogel capsules are placed into a subject in need thereof, such as a clotting factor, growth factor, hormone, enzyme, cytokine (e.g., a pro-inflammatory cytokine or an anti-inflammatory cytokine), cytokine receptor, chimeric protein, fusion protein or lipoprotein. The polypeptide encoded by the expression vector may have a naturally occurring amino acid sequence or may contain a variant of the naturally occurring sequence. The variant can be a non-naturally occurring or naturally occurring amino acid substitution, mutation, deletion or addition relative to the reference (e.g., naturally occurring) sequence. The naturally occurring amino acid sequence may be a polymorphic variant. The naturally occurring amino acid sequence can be a human or a non-human amino acid sequence. In some embodiments, the naturally occurring amino acid sequence is a human sequence. In some embodiments, the therapeutic polypeptide has about 2, 3, 4, 5, 6, 7, 8, 9, 10, 12, 14, 16, 18, 20, 25, 30, 35, 40, 45, or less than 50 amino acids. In some embodiments, the polypeptide has an average molecular weight of 5 kD, 10 kD, 25 kD, 50 kD, 100 kD, 150 kD, 200 kD, 250 kD, 500 kD, or more.

In some embodiments, the polypeptide is a hormone. Exemplary hormones include anti-diuretic hormone (ADH), oxytocin, growth hormone (GH), prolactin, growth hormone-releasing hormone (GHRH), thyroid stimulating hormone (TSH), thyrotropin-release hormone (TRH), adrenocorticotropic hormone (ACTH), follicle-stimulating hormone (FSH), luteinizing hormone (LH), luteinizing hormone-releasing hormone (LHRH), thyroxine, calcitonin, parathyroid hormone (PTH), aldosterone, cortisol, epinephrine, glucagon, insulin (INS), estrogen, progesterone, and testosterone. In some embodiments, the polypeptide is INS (e.g., insulin A-chain, insulin B-chain, or proinsulin). In some embodiments, the polypeptide is a growth hormone, such as human growth hormone (hGH), recombinant human growth hormone (rhGH), bovine growth hormone, methionine-human growth hormone, des-phenylalanine human growth hormone, and porcine growth hormone.

In some embodiments, the polypeptide is a growth factor, e.g., vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and -II (IGF-I and IGF-II).

In some embodiments, the polypeptide is a clotting factor or a coagulation factor, e.g., a blood clotting factor or a blood coagulation factor. In some embodiments, the polypeptide is involved in coagulation, i.e., the process by which blood is converted from a liquid to solid or gel. Exemplary clotting factors and coagulation factors include Factor I (e.g., fibrinogen), Factor II (e.g., prothrombin), Factor III (e.g., tissue factor), Factor V (e.g., proaccelerin, labile factor), Factor VI, Factor VII (e.g., stable factor, proconvertin), Factor VIII (e.g., antihemophilic factor A), Factor VIIIC, Factor IX (e.g., antihemophilic factor B), Factor X (e.g., Stuart-Prower factor), Factor XI (e.g., plasma thromboplastin antecedent), Factor XII (e.g., Hagerman factor), Factor XIII (e.g., fibrin-stabilizing factor), von Willebrand factor (vWF), prekallikrein, heparin cofactor II, high molecular weight kininogen (e.g., Fitzgerald factor), antithrombin III, and fibronectin. In some embodiments, the polypeptide is an anti-clotting factor, such as Protein C.

In some embodiments, the polypeptide is an immunoglobulin chain (heavy or light chain) or fragment thereof, comprising at least one immunoglobulin variable domain sequence, and optionally comprising an immunoglobulin Fc region. In an embodiment, the polypeptide a full-length immunoglobulin chain.

In some embodiments, the polypeptide is a cytokine or a cytokine receptor, or a chimeric protein including cytokines or their receptors, including, for example tumor necrosis factor alpha and beta, their receptors and their derivatives, renin; lipoproteins; colchicine; corticotrophin; vasopressin; somatostatin; lypressin; pancreozymin; leuprolide; alpha-1-antitrypsin; atrial natriuretic factor; lung surfactant; a plasminogen activator other than a tissue-type plasminogen activator (t-PA), for example a urokinase; bombesin; thrombin; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; chorionic gonadotropin; a microbial protein, such as beta-lactamase; DNase; inhibin; activin; receptors for hormones or growth factors; integrin; protein A or D; rheumatoid factors; platelet-derived growth factor (PDGF); epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; an interferon such as interferon-alpha (e.g., interferon.alpha.2A), -beta, -gamma, -lambda and consensus interferon; colony stimulating factors (CSFs), e.g., M-CSF, GM-CSF, and G-CSF; interleukins (TLs), e.g., IL-1, IL-2 to IL-10; superoxide dismutase; T-cell receptors; surface membrane proteins; decay accelerating factor; transport proteins; homing receptors; addressins; fertility inhibitors such as the prostaglandins; fertility promoters; regulatory proteins; antibodies (including fragments thereof) and chimeric proteins, such as immunoadhesins. Suitable polypeptides may be native or recombinant and include, e.g., fusion proteins.

Examples of a polypeptide that may be encoded by the exogenous transcription unit also include CCL1, CCL2 (MCP-1), CCL3 (MIP-1a), CCL4 (MIP-10), CCL5 (RANTES), CCL6, CCL7, CCL8, CCL9 (CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1 (KC), CXCL2 (SDF1a), CXCL3, CXCL4, CXCL5, CXCL6, CXCL7, CXCL8 (IL8), CXCL9, CXCL10, CXCL11, CXCL12, CXCL13, CXCL14, CXCL15, CXCL16, CXCL17, CX3CL1, XCL1, XCL2, TNFA, TNFB (LTA), TNFC (LTB), TNFSF4, TNFSF5 (CD40LG), TNFSF6, TNFSF7, TNFSF8, TNFSF9, TNFSF10, TNFSF11, TNFSF13B, EDA, IL2, IL15, IL4, IL13, IL7, IL9, IL21, IL3, IL5, IL6, IL11, IL27, IL30, IL31, OSM, LIF, CNTF, CTF1, IL12a, IL12b, IL23, IL27, IL35, IL14, IL16, IL32, IL34, IL10, IL22, IL19, IL20, IL24, IL26, IL29, IFNL1, IFNL2, IFNL3, IL28, IFNA1, IFNA2, IFNA4, IFNA5, IFNA6, IFNA7, IFNA8, IFNA10, IFNA13, IFNA14, IFNA16, IFNA17, IFNA21, IFNB1, IFNK, IFNW1, IFNG, IL1A (IL1F1), IL1B (IL1F2), IL1Ra (IL1F3), IL1F5 (IL36RN), IL1F6 (IL36A), IL1F7 (IL37), IL1F8 (IL36B), IL1F9 (IL36G), IL1F10 (IL38), IL33 (IL1F11), 1118 (IL1G), IL17, KITLG, IL25 (IL17E), CSF1 (M-CSF), CSF2 (GM-CSF), CSF3 (G-CSF), SPP1, TGFB1, TGFB2, TGFB3, CCL3L¹, CCL3L2, CCL3L3, CCL4L1, CCL4L2, IL17B, IL17C, IL7D, IL7F, AIMP1 (SCYE1), MIF, Areg, BC096441, Bmp1, Bmp10, Bmp15, Bmp2, Bmp3, Bmp4, Bmp5, Bmp6, Bmp7, Bmp8a, Bmp8b, C1qtnf4, Ccl21a, Ccl27a, Cd70, Cer1, Cklf, Clcf1, Cmtm2a, Cmtm2b, Cmtm3, Cmtm4, Cmtm5, Cmtm6, Cmtm7, Cmtm8, Crlf1, Ctf2, Ebi3, Edn1, Fam3b, Fasl, Fgf2, Flt31, Gdf10, Gdf11, Gdf15, Gdf2, Gdf3, Gdf5, Gdf6, Gdf7, Gdf9, Gm12597, Gm13271, Gm13275, Gm13276, Gm13280, Gm13283, Gm2564, Gpi1, Grem1, Grem2, Grn, Hmgb1, Ifna11, Ifna12, Ifna9, Ifnab, Ifne, Il17a, Il23a, Il25, Il31, Iltifb, Inhba, Lefty1, Lefty2, Mstn, Nampt, Ndp, Nodal, Pf4, Pglyrpl, Prl7d1, Scg2, Scgb3a1, Slurp1, Spp1, Thpo, Tnfsf10, Tnfsf11, Tnfsf12, Tnfsf13, Tnfsf13b, Tnfsf14, Tnfsf15, Tnfsf18, Tnfsf4, Tnfsf8, Tnfsf9, Tslp, Vegfa, Wnt1, Wnt2, Wnt5a, Wnt7a, Xcl1, epinephrine, melatonin, triiodothyronine, a prostaglandin, a leukotriene, prostacyclin, thromboxane, islet amyloid polypeptide, mullerian inhibiting factor or hormone, adiponectin, corticotropin, angiotensin, vasopressin, arginine vasopressin, atriopeptin, brain natriuretic peptide, calcitonin, cholecystokinin, cortistatin, enkephalin, endothelin, erythropoietin, follicle-stimulating hormone, galanin, gastric inhibitory polypeptide, gastrin, ghrelin, glucagon, glucagon-like peptide-1, gonadotropin-releasing hormone, hepcidin, human chorionic gonadotropin, human placental lactogen, inhibin, somatomedin, leptin, lipotropin, melanocyte stimulating hormone, motilin, orexin, oxytocin, pancreatic polypeptide, pituitary adenylate cyclase-activating peptide, relaxin, renin, secretin, somatostatin, thrombopoietin, thyrotropin, thyrotropin-releasing hormone, vasoactive intestinal peptide, androgen, alpha-glucosidase (also known as acid maltase), glycogen phosphorylase, glycogen debrancher enzyme, phosphofructokinase, phosphoglycerate kinase, phosphoglycerate mutase, lactate dehydrogenase, carnitine palymityl transferase, carnitine, and myoadenylate deaminase.

In some embodiments, the polypeptide is a replacement therapy or a replacement protein.

In some embodiments, the replacement therapy or replacement protein is an enzyme, e.g., alpha-galactosidase A (GLA), alpha-L-iduronidase (IDUA), arylsulfatase B (ARSB), glucocerebrosidase, or N-sulfoglucosamine sulfohydrolase (SGSH).

In some embodiments, the replacement therapy or replacement protein is a clotting factor or a coagulation factor, e.g., Factor VII, Factor VIII (e.g., comprises a naturally occurring human Factor VIII amino acid sequence or a variant thereof) or Factor IX (e.g., comprises a naturally occurring human Factor IX amino acid sequence or a variant thereof).

In an embodiment, the genetically modified cells have one or more of the following characteristics: (i) are not capable of producing INS (e.g., insulin A-chain, insulin B-chain, or proinsulin) in an amount effective to treat diabetes or another disease or condition that may be treated with INS; (ii) not capable of producing INS in a glucose-responsive manner; or (iii) not derived from an induced pluripotent stem cell that was engineered or differentiated into INS-producing pancreatic beta cells.

In an embodiment, the genetically modified cells have one or more of the following characteristics: (i) are capable of producing INS (e.g., insulin A-chain, insulin B-chain, or proinsulin) in an amount effective to treat diabetes or another disease or condition that may be treated with INS; (ii) are capable of producing INS in a glucose-responsive manner; or (iii) derived from an induced pluripotent stem cell that was engineered or differentiated into INS-producing pancreatic beta cells.

### Storage and Shipping

The hydrogel capsule composition may be provided in a sealed container. In some embodiments, all interior container surfaces in contact with the composition consist essentially of, or consist of, a chemically and biologically inert material. Suitable materials include a pharmaceutical-grade or medical-grade plastic, e.g., polycarbonate, polyetherimide (PEI), polyethylene, polypropylene, polyvinyl chloride, PEEK, polysulfone, polyurethane, fluorinated ethylene propylene (FEP) or polyethylene terephthalate glycol (PETG).

In an embodiment, the container may be of any size and shape that allows the container to be stored in a manner that allows a substantially uniform capsule layer containing substantially all of the hydrogel capsules in the composition to form across the bottom interior surface of the stored container and a layer of the aqueous solution that is substantially free of hydrogel capsules forms on top of the capsule layer. In some embodiments, the capsule layer has a depth equal to about 1.00 to about 1.25 times the mean diameter of the capsules in the composition. In some embodiments, the total volume of the aqueous solution (VS) in the container (e.g., amount of residual solution within the capsule layer plus the amount in the solution layer) is equal to or greater than the volume of hydrogel capsules in the container. In some embodiments, the ratio of the total volume of the aqueous solution to the total volume of hydrogel capsules in the container is between about any of 1, 1.1, 1.2, 1.3, 1.4 or 1.5 and about 100, between about 2 and about 75, between about 3 and about 50, between about 4 and about 40, between about 5 and about 30, and between about 10 and about 20. In an embodiment, the mean capsule diameter of the hydrogel capsules in the container is about 1500 μm and the ratio of VHC to VS is at least about 1:1, about 1:2, or about 1:3 and less than about 1:40, e.g., about any of 1:1, 1:2, 1:5, 1:10, 1:15, 1:20, 1:25, 1:30, and 1:35.

In some embodiments, the configuration (size/shape) of the container and the volume of the composition placed in the container are selected to allow an air space between the top of the solution layer and the upper interior surface of the stored container. In some embodiments, the air space is at least about 0.5 inches to 2.5 inches, about 1.0 inches to 2.0 inches, or about 1.5 inches to 2.0 inches.

In an embodiment, the storage container is a flexible, rectangular bag made of FEP, with one or more ports configured to allow addition of the composition or other material(s) to the bag (e.g., via a syringe) and one or more ports configured to remove solution with or without hydrogel capsules from the bag (e.g., via a syringe. An exemplary FEP bag has an interior surface area (2×the bag face) of about 400 cm² to about 600 cm² with a maximum fill volume of about 400 milliliter (mL) to about 700 mL. In an embodiment, the amount of the hydrogel capsule composition in the FEP bag is between about 200 ml to 500 ml, e.g., between about any of 300 to 500 ml, 400 to 500 ml, 200 to 400 ml, 200 to 300 ml, 300 to 400 ml. Other exemplary containers for storing a hydrogel capsule composition described herein include a round-bottom bottle formed from PETG sealed with a cap and a rectangular tray formed from PETG and sealed with a foil lid.

The selection of the amount of hydrogel capsules and aqueous solution to add to a container of a particular size will depend upon the size of the capsules, the number of capsules per mL of the capsule composition, the desired VHC to VS ratio, as well as any desired air space above the solution layer. These amounts can be determined by the skilled person using mathematical formulas known in the art. An exemplary approach to determine the volume of spherical shaped capsules (“spheres”) to add to a container is shown below.
a. Calculate the surface area covered by the spheres by multiplying the surface area of the container base times an expected sphere packing target, e.g., 0.64 for random packing to 0.74 for perfect packing;
b. Calculate the surface area of a sphere with this formula: (Mean sphere diameter/2)²*Pi
c. Calculate number of spheres to add to the container by dividing the result of step a (surface area covered by spheres) by the result of step b (surface area of a sphere); and
d. Divide the result of step c by the number of spheres per mL of the composition.
   The table immediately below sets forth exemplary volumes of sphere preparations that would be added to an exemplary FEP bag with interior surface area of about 500 cm² and a random packing target (0.64) for three different mean sphere diameters (1000 micrometers (μm), 1400 micrometers (μm) and 2000 μm) and two different sphere concentrations for each diameter.

| Sphere | | mL of Sphere Preparation |
|---|---|---|
| Diameter (μm) | Spheres/mL | in Container |
| 1000 | 200 | 204 |
| 1000 | 400 | 102 |
| 1400 | 200 | 104 |
| 1400 | 400 | 52 |
| 2000 | 200 | 51 |
| 2000 | 400 | 25 |

In some embodiments of a capsule composition in which the population of hydrogel capsules consists essentially of spherical shaped capsules (“spheres”), the mean sphere diameter in the population represents the mean of the diameters of at least 10 spheres. In an embodiment, the diameters of the at least ten spheres are obtained by analysis of a microscopic image of an aliquot of the hydrogel capsule composition.

In an embodiment, after the capsule composition has been added to the container, the container is sealed, and then stored between about 2° C. and room temperature (15° C. to 25° C.; 59° F. to 77° F.) for a desired time period, which time period may include the time required to ship the container to a patient implantation site, e.g., the operating room in a hospital. In an embodiment, the desired time period is any of 12 hours, 24 hours, 36 hours, 2 days, 3 days, 4 days or 5 days. In an embodiment, at the end of the desired time period, the encapsulated cells in the sealed container retain at least 80%, 85%, 90%, 95% or up to 100% of their starting viability and/or productivity, which may be assessed using any method known in the art or described herein.

### Methods of Treatment

A hydrogel capsule composition described herein may be administered to a patient in need of treatment with a therapeutic substance secreted by the encapsulated cells. In an embodiment, the capsule composition, or a therapeutically effective amount thereof, is administered to, implanted in, or otherwise disposed into the peritoneal cavity (e.g., the omentum), which site of administration may include one or more of the lesser sac, also known as the omental bursa or bursalis omentum. The lesser sac refers to a cavity located in the abdomen formed by the omentum, and is in close proximity to, for example, the greater omentum, lesser omentum, stomach, small intestine, large intestine, liver, spleen, gastrosplenic ligament, adrenal glands, and pancreas. Typically, the lesser sac is connected to the greater sac via the omental foramen (i.e., the Foramen of Winslow). A therapeutically effective amount of a hydrogel capsule composition may be implanted in the peritoneal cavity (e.g., the omentum, e.g., the lesser sac) or disposed on a surface within the peritoneal cavity (e.g., omentum, e.g., lesser sac) via injection or catheter. Additional considerations for implantation into the omentum (e.g., the lesser sac) are provided in M.

Pellicciaro et al. (2017) CellR4 5(3):e2410, which is incorporated herein by reference in its entirety.

### ENUMERATED EMBODIMENTS

1. A composition comprising a population of hydrogel capsules disposed in a pharmaceutically acceptable aqueous solution, wherein each hydrogel capsule in the population encapsulates a plurality of live mammalian cells, wherein the solution has a pH of between 6.0 and 9.0 at 12° C. to 30° C. (e.g., about 15-25° C.), and comprises a calcium salt at an elemental calcium concentration of between about 1.0 millimolar (mM) and about 10 mM.
2. The composition of embodiment 1, wherein the solution has an osmolality of about 250 milliosmole/kg solution to about 350 milliosmole/kg solution.
3. The composition of any one of the preceding embodiments, wherein each hydrogel capsule in the population comprises an ionically cross-linked alginate.
4. The composition of any one of the preceding embodiments, wherein the pH of the solution is between 6.5 and 9.0 at 15° C. to 25° C.
5. The composition of any one of the preceding embodiments, wherein the elemental calcium concentration is between an x value and a y value, wherein the x and y values are selected from the group consisting of:
   (i) x=about 1.1 mM and y=about 8.0 mM, 6.0 mM, 4.0 mM, or 2.0 mM;
   (ii) x=about 1.2 mM and y=about 5 mM, 4 mM, 3 mM, or 2.0 mM;
   (iii) x=about 1.2 mM and y=about 2.0 mM or 1.5 mM;
   (iv) x=about 1.3 mM and y=about 1.4 mM;
   (v) x=about 1.4 mM and y=about 4.0. mM, 3.0 mM, or 2.0 mM; and
   (vi) x=about 1.5 mM and y=about 2.5 mM.
6. The composition of any one of the preceding embodiments, wherein the solution further comprises at least one carbon source (e.g., a sugar (e.g., dextrose, glucose, galactose, hexose, fructose, maltose), glycerol, glutamine, pyruvate or salt thereof).
7. The composition of any one of the preceding embodiments, wherein the solution further comprises a buffering agent which comprises one or more of an acetate salt (e.g., sodium acetate), a gluconate salt (e.g., sodium gluconate), a phosphate salt (e.g., sodium phosphate monobasic), a bicarbonate salt (e.g., sodium bicarbonate), and a lactate salt (e.g., sodium lactate).
8. The composition of embodiment 7, wherein the buffering agent comprises sodium acetate and sodium gluconate.
9. The composition of embodiment 8, wherein the buffering agent consists essentially of about 0.5-5 g/L sodium acetate (e.g., 2 g/L, e.g., 2.29 g/L sodium acetate) and about 0.5-10 g/L sodium gluconate (e.g., 5 g/L, e.g., 5.18 g/L) sodium gluconate.
10. The composition of embodiment 6, wherein the carbon source is glucose, and the solution does not contain any added glutamine or phenol red.
11. The composition of embodiment 7, wherein the buffering agent comprises sodium bicarbonate and sodium phosphate and the solution does not contain any added HEPES or sodium pyruvate.
12. The composition of any one of the preceding embodiments, wherein the calcium salt is calcium chloride.
13. The composition of any one of the preceding embodiments, wherein the solution comprises about 1.5 mM to 2.5 mM calcium chloride, about 5 mM to about 25 mM D-glucose, and about 40 mM to about 50 mM sodium bicarbonate.
14. The composition of any one of the preceding embodiments, wherein the solution further comprises:
   (i) a magnesium compound (e.g., magnesium chloride or magnesium sulfate);
   (ii) a potassium compound (e g potassium chloride);
   (iii) sodium chloride; and
   (iv) a set of amino acids, which comprises histidine, isoleucine, leucine, lysine, methionine, phenylalanine, threonine, tryptophan, and valine; and
   (v) a set of vitamins, which comprises a vitamin B1 compound (e.g., thiamine or a thiamine salt, e.g., thiamine hydrochloride), a vitamin B3 compound (e.g., nicotinic acid or niacinamide) and a vitamin B6 compound (e.g., pyroxidine or a pyroxidine salt, e.g., pyroxidine hydrochloride).
15. The composition of claim 14, wherein the set of amino acids also comprises arginine, glycine, cystine, serine, and tyrosine and the set of vitamins also comprises choline or a choline salt (e.g., choline chloride), a vitamin B5 compound (e.g., pantotheneic acid or calcium pantothenate), a folate compound (e.g., folic acid), riboflavin and i-inositol.
16. The composition of any one of the preceding embodiments, where some or all of the amino acids in the set are L-amino acids.
17. The composition of any one of the preceding embodiments, wherein the solution comprises or consists essentially of the components and concentrations set forth immediately below:

| Component | Concentration (g/L) |
|---|---|
| Calcium chloride dihydrate | About 0.298 |
| Sodium chloride | About 5.252 |
| Sodium acetate | About 2.291 |
| Sodium gluconate | About 5.175 |
| Potassium chloride | About 0.3703 |
| Magnesium chloride hexahydrate | About 0.302 |
| Water | Not applicable |

18. The composition ofany one of the preceding embodiments, wherein the solution comprises or consists essentially of the components and concentrations set forth immediately below:

| Component | Concentration (mM) |
|---|---|
| Glycine | About 0.400 |
| L-Arginine | About 0.398 |
| hydrochloride | |
| L-Cystine | About 0.201 |
| dihydrochloride | |
| L-Histidine | About 0.200 |
| monohydrochloride | |
| monohydrate | |
| L-Isoleucine | About 0.802 |
| L-Leucine | About 0.802 |
| L-Lysine | About 0.798 |
| hydrochloride | |
| L-Methionine | About 0.201 |
| L-Phenylalanine | About 0.400 |
| L-Serine | About 0.400 |
| L-Tryptophan | About 0.078 |
| L-Tyrosine | About 0.398 |
| disodium salt | |
| dihydrate | |
| L-Valine | About 0.803 |
| Choline chloride | About 0.029 |
| D-Calcium | About 0.008 |
| pantothenate | |
| Folic acid | About 0.009 |
| Niacinamide | About 0.038 |
| Pyridoxine | About 0.018 |
| hydrochloride | |
| Riboflavin | About 0.001 |
| Thiamine | About 0.012 |
| hydrochloride | |
| i-Inositol | About 0.040 |
| Calcium chloride | About 1.802 |
| (anhyd.) | |
| Ferric nitrate | About 2.475 × 10⁻⁴ |
| nonahydrate | |
| Magnesium sulfate | About 0.814 |
| (anhyd.) | |
| Potassium chloride | About 5.333 |
| Sodium | About 44.048 |
| bicarbonate | |
| Sodium chloride | About 110.345 |
| Sodium phosphate | About 0.906 |
| monobasic | |
| Water | Not applicable |

optionally wherein the solution further comprises D-glucose in an amount selected from the groups consisting of:
about 1 mM to about 50 mM;
about 2 mM to about 40 mM;
about 3 mM to about 30 mM;
about 4 mM to about 20 mM;
about 5 mM to about 10 mM;
about 10 mM to about 40 mM;
about 15 mM to about 35 mM;
about 20 mM to about 30 mM; and
about 25 mM.
19. The composition of any one of the preceding embodiments, wherein each hydrogel capsule in the population has a sphere-like or spherical shape and comprises:
(a) a cell-containing compartment which comprises the plurality of live cells encapsulated in a first polymer composition; and
(b) a barrier compartment surrounding the cell-containing compartment and comprising a second polymer composition which comprises the ionically cross-linked alginate;
wherein the mean diameter of the hydrogel capsules in the population is about 500 micrometers (μm) to about 5000 μm, about 1000 (μm) to about 3000 μm, about 1100 μm to about 2500 μm, about 1200 μm to about 2300 μm, about 1300 μm to about 2100 μm, about 1400 μm to about 2000 μm, about 1400 μm to about 1900 μm, about 1400 μm to about 1800 μm.
20. The composition of embodiment 19, wherein the mean capsule diameter of the hydrogel capsules in the population is 1400 to 2000 μm.
21. The composition of any one of the preceding embodiments, wherein the average thickness of the barrier compartment is about 10 to about 300 microns, about 20 to about 150 microns, or about 40 to about 75 microns.
22. The composition of any one of the preceding embodiments, wherein the first polymer composition comprises an alginate covalently modified with a cell-contacting peptide via a linker and wherein the cross-linked alginate in the barrier compartment comprises an alginate covalently modified with at least one afibrotic compound, optionally a compound selected from the compounds shown in the table below:

| Compound | |
|---|---|
| No. | Structure |
| 100 | 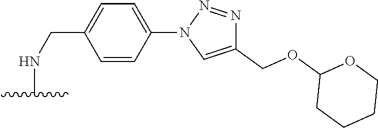 [SMILES: *NCc1ccc(-n2cc(COC3CCCCO3)nn2)cc1] |
| 101 | 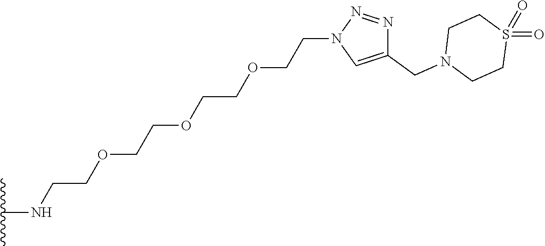 [SMILES: *NCCOCCOCCOCCn1cc(CN2CCS(=O)(=O)CC2)nn1] |
| 102 | 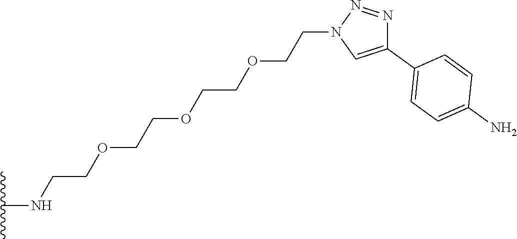 [SMILES: *NCCOCCOCCOCCn1cc(-c2ccc(N)cc2)nn1] |
| 103 | 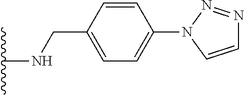 [SMILES: *NCc1ccc(-n2ccnn2)cc1] |
| 104 | 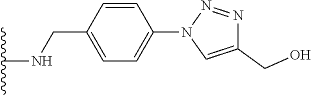 [SMILES: *NCc1ccc(-n2cc(CO)nn2)cc1] |
| 105 | 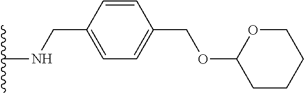 [SMILES: *NCc1ccc(COC2CCCCO2)cc1] |
| 106 | 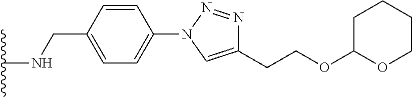 [SMILES: *NCc1ccc(-n2cc(CCOC3CCCCO3)nn2)cc1] |
| 107 | 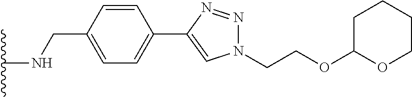 [SMILES: *NCc1ccc(-c2cn(CCOC3CCCCO3)nn2)cc1] |
| 108 | 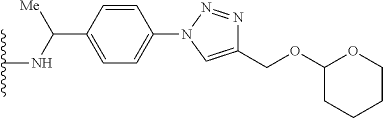 [SMILES: *NC(C)c1ccc(-n2cc(COC3CCCCO3)nn2)cc1] |
| 109 | 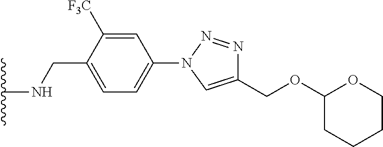 [SMILES: *NCc1ccc(-n2cc(COC3CCCCO3)nn2)cc1C(F)(F)F] |
| 110 | 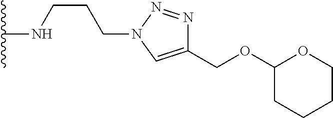 [SMILES: *NCCCn1cc(COC2CCCCO2)nn1] |
| 111 | 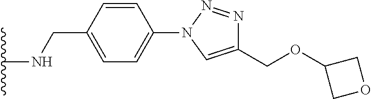 [SMILES: *NCc1ccc(-n2cc(COC3COC3)nn2)cc1] |
| 112 | 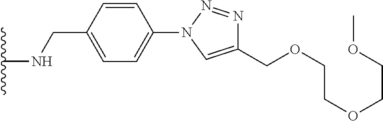 [SMILES: *NCc1ccc(-n2cc(COCCOCCOC)nn2)cc1] |
| 113 | 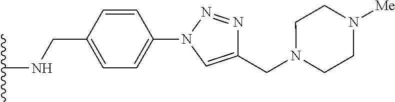 [SMILES: *NCc1ccc(-n2cc(CN3CCN(C)CC3)nn2)cc1] |
| 114 | 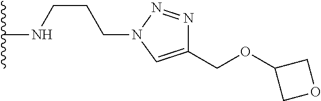 [SMILES: *NCCCn1cc(COC2COC2)nn1] |
| 115 | 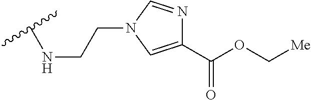 [SMILES: *NCCn1cnc(C(=O)OCC)c1] |
| 116 | 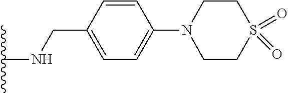 [SMILES: *NCc1ccc(N2CCS(=O)(=O)CC2)cc1] |
| 117 | 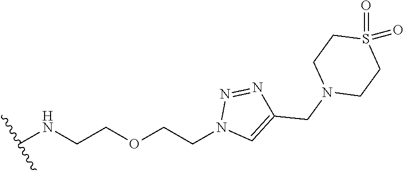 [SMILES: *NCCOCCn1cc(CN2CCS(=O)(=O)CC2)nn1] |
| 118 | 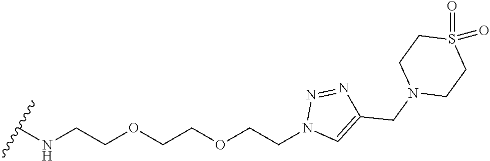 [SMILES: *NCCOCCOCCn1cc(CN2CCS(=O)(=O)CC2)nn1] |
| 119 | 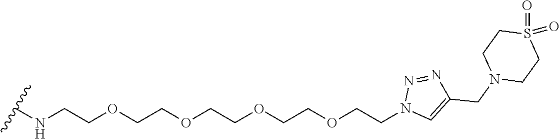 [SMILES: *NCCOCCOCCOCCOCCn1cc(CN2CCS(=O)(=O)CC2)nn1] |
| 120 | 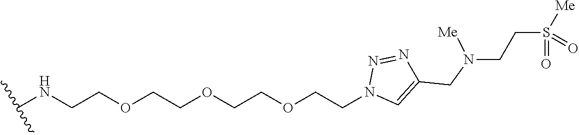 [SMILES: *NCCOCCOCCOCCn1cc(CN(C)CCS(C)(=O)=O)nn1] |
| 121 | 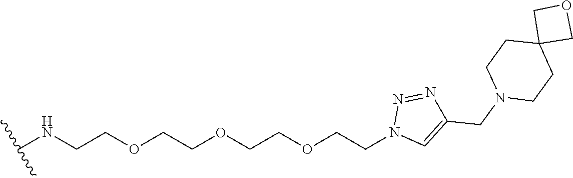 [SMILES: *NCCOCCOCCOCCn1cc(CN2CCC3(CC2)COC3)nn1] |
| 122 | 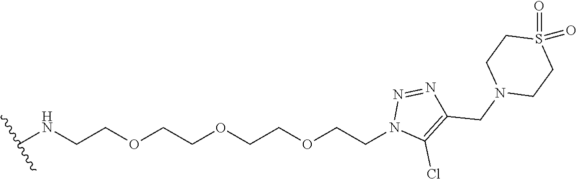 [SMILES: *NCCOCCOCCOCCn1nnc(CN2CCS(=O)(=O)CC2)c1Cl] |
| 123 | 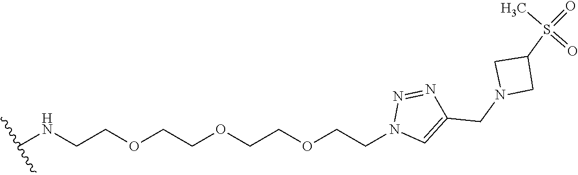 [SMILES: *NCCOCCOCCOCCn1cc(CN2CC(S(C)(=O)=O)C2)nn1] |
| 124 | 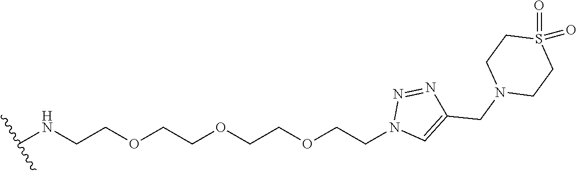 [SMILES: *NCCOCCOCCOCCn1cc(CN2CCS(=O)(=O)CC2)nn1] |
| 125 | 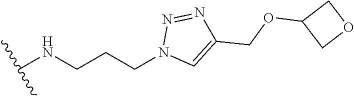 [SMILES: *NCCCn1cc(COC2COC2)nn1] |
| 126 | 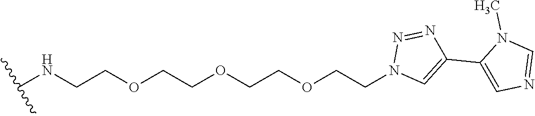 [SMILES: *NCCOCCOCCOCCn1cc(-c2cncn2C)nn1] |
| 127 | 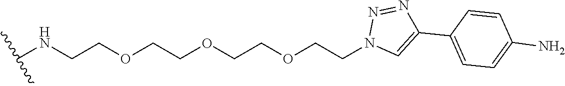 [SMILES: *NCCOCCOCCOCCn1cc(-c2ccc(N)cc2)nn1] |
| 128 | 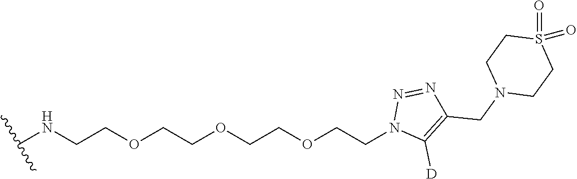 [SMILES: *NCCOCCOCCOCCn1nnc(CN2CCS(=O)(=O)CC2)c1[2H]] |
| 129 | 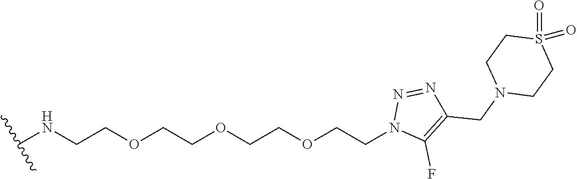 [SMILES: *NCCOCCOCCOCCn1nnc(CN2CCS(=O)(=O)CC2)c1F] |
| 130 | 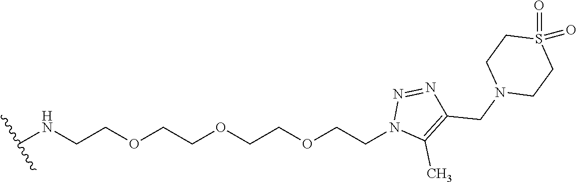 [SMILES: *NCCOCCOCCOCCn1nnc(CN2CCS(=O)(=O)CC2)c1C] |
| 131 | 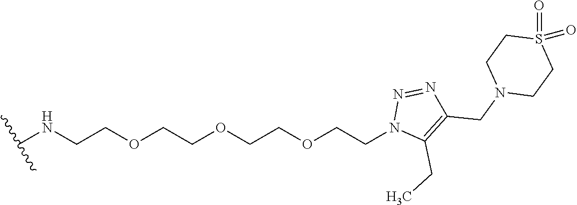 [SMILES: *NCCOCCOCCOCCn1nnc(CN2CCS(=O)(=O)CC2)c1CC] |
| 132 | 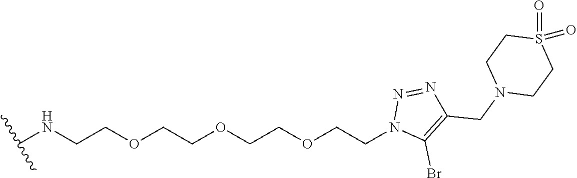 [SMILES: *NCCOCCOCCOCCn1nnc(CN2CCS(=O)(=O)CC2)c1Br] |
| 133 | 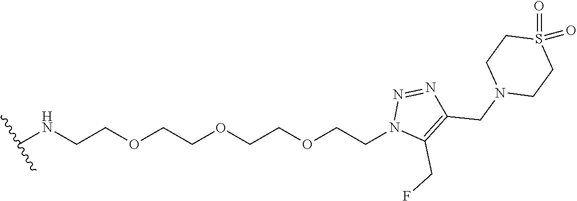 [SMILES: *NCCOCCOCCOCCn1nnc(CN2CCS(=O)(=O)CC2)c1CF] |
| 134 | 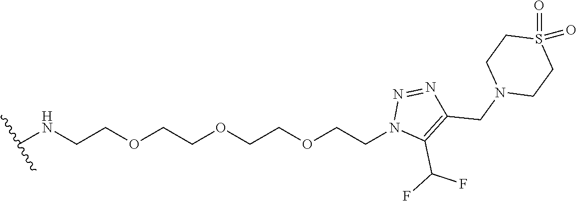 [SMILES: *NCCOCCOCCOCCn1nnc(CN2CCS(=O)(=O)CC2)c1C(F)F] |
| 135 | 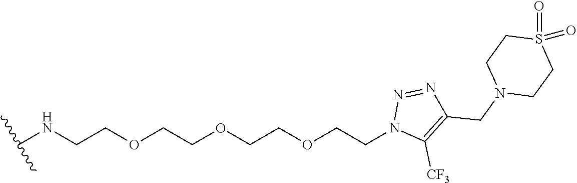 [SMILES: *NCCOCCOCCOCCn1nnc(CN2CCS(=O)(=O)CC2)c1C(F)(F)F] |
| 136 | 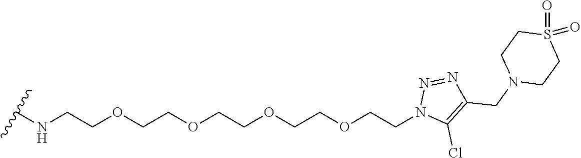 [SMILES: *NCCOCCOCCOCCOCCn1nnc(CN2CCS(=O)(=O)CC2)c1Cl] |
| 137 | 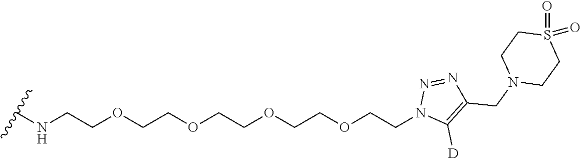 [SMILES: *NCCOCCOCCOCCOCCn1nnc(CN2CCS(=O)(=O)CC2)c1[2H]] |
| 138 | 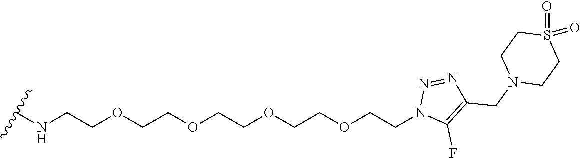 [SMILES: *NCCOCCOCCOCCOCCn1nnc(CN2CCS(=O)(=O)CC2)c1F] |
| 139 | 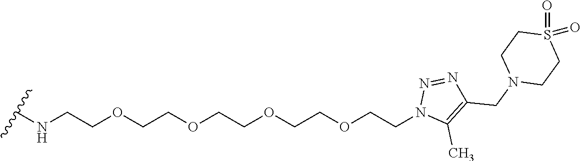 [SMILES: *NCCOCCOCCOCCOCCn1nnc(CN2CCS(=O)(=O)CC2)c1C] |
| 140 | 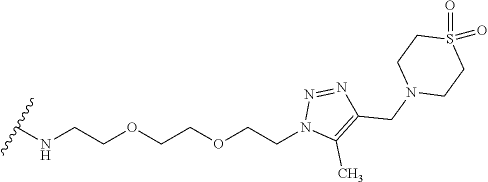 [SMILES: *NCCOCCOCCn1nnc(CN2CCS(=O)(=O)CC2)c1C] |
| 141 | 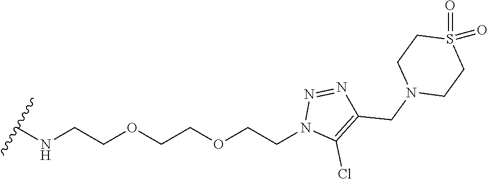 [SMILES: *NCCOCCOCCn1nnc(CN2CCS(=O)(=O)CC2)c1Cl] |
| 142 | 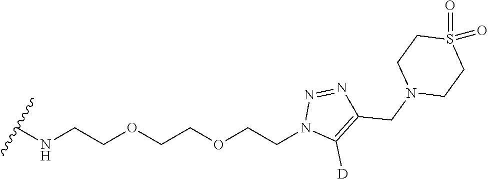 [SMILES: *NCCOCCOCCn1nnc(CN2CCS(=O)(=O)CC2)c1[2H]] |
| 143 | 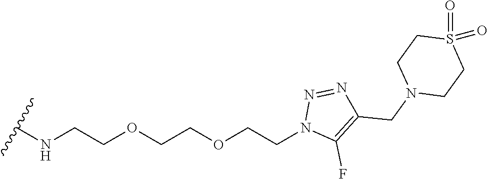 [SMILES: *NCCOCCOCCn1nnc(CN2CCS(=O)(=O)CC2)c1F] |
| 144 | 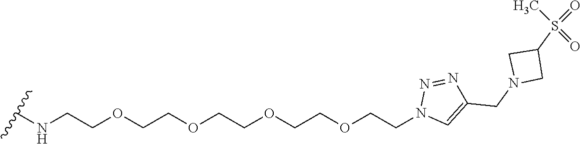 [SMILES: *NCCOCCOCCOCCOCCn1cc(CN2CC(S(C)(=O)=O)C2)nn1] |
| 145 | 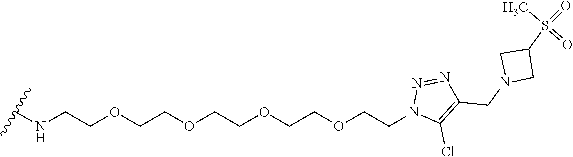 [SMILES: *NCCOCCOCCOCCOCCn1nnc(CN2CC(S(C)(=O)=O)C2)c1Cl] |
| 146 | 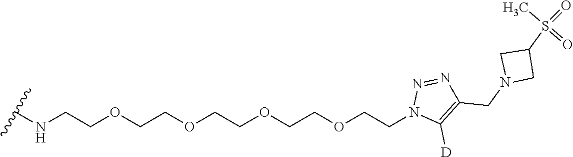 [SMILES: *NCCOCCOCCOCCOCCn1nnc(CN2CC(S(C)(=O)=O)C2)c1[2H]] |
| 147 | 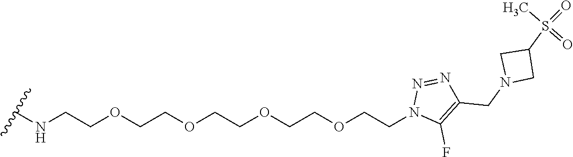 [SMILES: *NCCOCCOCCOCCOCCn1nnc(CN2CC(S(C)(=O)=O)C2)c1F] |
| 148 | 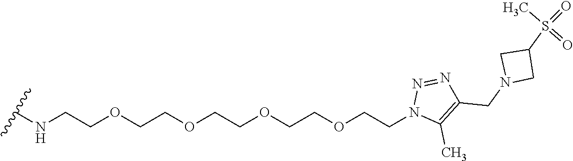 [SMILES: *NCCOCCOCCOCCOCCn1nnc(CN2CC(S(C)(=O)=O)C2)c1C] |
| 149 | 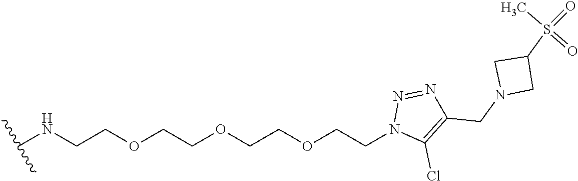 [SMILES: *NCCOCCOCCOCCn1nnc(CN2CC(S(C)(=O)=O)C2)c1Cl] |
| 150 | 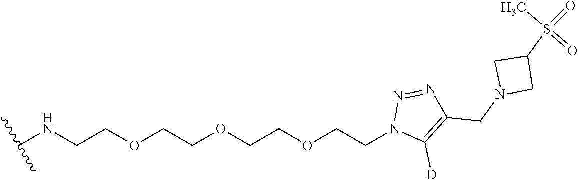 [SMILES: *NCCOCCOCCOCCn1nnc(CN2CC(S(C)(=O)=O)C2)c1[2H]] |
| 151 | 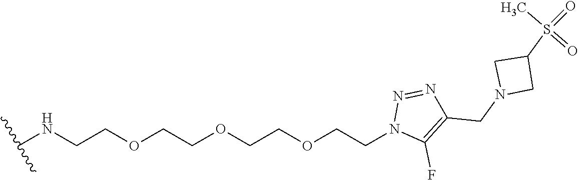 [SMILES: *NCCOCCOCCOCCn1nnc(CN2CC(S(C)(=O)=O)C2)c1F] |
| 152 | 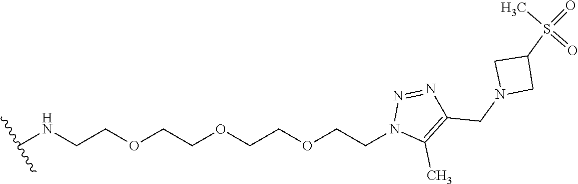 [SMILES: *NCCOCCOCCOCCn1nnc(CN2CC(S(C)(=O)=O)C2)c1C] |
| 153 | 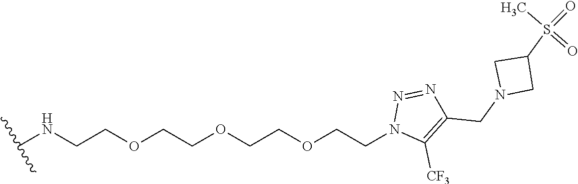 [SMILES: *NCCOCCOCCOCCn1nnc(CN2CC(S(C)(=O)=O)C2)c1C(F)(F)F] |
| 154 | 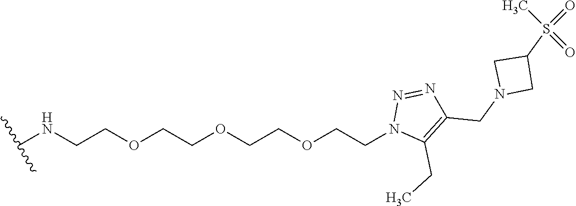 [SMILES: *NCCOCCOCCOCCn1nnc(CN2CC(S(C)(=O)=O)C2)c1CC] |

23. The composition of embodiment 21, wherein the ionically cross-linked alginate in the barrier compartment comprises barium ions as at least one cross-linking agent.
24. The composition of any one of the preceding embodiments, wherein the ionically cross-linked alginate in the barrier compartment comprises a mixture of the covalently modified alginate and an unmodified alginate.
25. The composition of any one of the preceding embodiments, wherein the covalently modified alginate in the cell-containing compartment is ionically cross-linked with barium ions as at least one cross-linking agent.
26. The composition of any one of the preceding embodiments, wherein:
   (a) the mean diameter of the hydrogel capsules in the population is 1400 m to 2000 μm, or 1400 m to 1600 μm, or 1000 m to 1200 μm;
   (b) the alginate in the first polymer composition has a molecular weight of 150 to 250 kDa and a G:M ratio of greater than or equal to 1.5;
   (c) the cell-contacting peptide consists of RGDSP and the linker is a single glycine residue attached to the N-terminus of the cell-contacting peptide;
   (d) the alginate in the covalently-modified alginate in the barrier compartment has a molecular weight of <75 kDa and a G:M ratio of greater than or equal to 1.5;
   (e) the afibrotic compound is

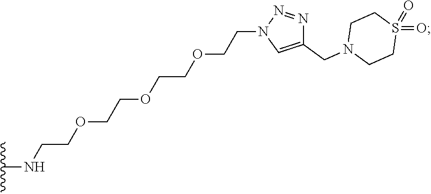 [SMILES: *NCCOCCOCCOCCn1cc(CN2CCS(=O)(=O)CC2)nn1]

and
   (f) the unmodified alginate in the barrier compartment has a molecular weight of 150 kDa to 250 kDa and a G:M ratio of greater than or equal to 1.5.
27. The composition of any one of the preceding embodiments, wherein the live mammalian cells are human cells.
28. The composition of any one of the preceding embodiments, wherein the cells are derived from an induced pluripotent stem cell.
29. The composition of any one of the preceding embodiments, wherein the cells are derived from an RPE cell, optionally derived from an ARPE-19 cell.
30. The composition of any one of the preceding embodiments, wherein the encapsulated cells comprise single cells.
31. The composition of any one of the preceding embodiments, wherein the encapsulated cells comprise one or more cell clusters.
32. The composition of any one of the preceding embodiments, wherein the encapsulated cells comprise cells disposed on a microbead.
33. The composition of any one of the preceding embodiments, wherein the live mammalian cells are genetically modified to express and secrete a therapeutic substance, e.g., a therapeutic polypeptide.
34. The composition of any one of the preceding embodiments, wherein the mammalian cells comprise an exogenous nucleotide sequence which encodes a therapeutic polypeptide, optionally wherein the therapeutic polypeptide is a growth factor, a blood coagulation factor, an enzyme, a cytokine, a cytokine receptor, an antibody or antigen-binding fragment thereof.
35. The composition of embodiment 34, wherein the therapeutic polypeptide is an FVIII protein (e.g., an FVIII BDD protein), a FIX protein, or a FVII protein.
36. The composition of embodiment 34, wherein the therapeutic polypeptide is a GLA protein, an IDUA protein, an IDS protein, an ARSB protein, or a GBA protein.
37. The composition of any one of embodiments 19 to 36, wherein the plurality of live mammalian cells is between about 5,000 to about 250,000 cells, about 10,000 to about 125,000 cells, about 20,000 to about 75,000 cells, about 12,500 to about 40,000 cells, or about 15,000 to about 30,000 cells.
38. The composition of any one of the preceding embodiments, which comprises about 200 to about 400 of the hydrogel capsules per milliliter of the pharmaceutically acceptable solution.
39. A sealed container comprising the composition of any one of the above claims.
40. The sealed container of claim 39, wherein the volume of the aqueous solution (VS) in the container is about equal to or greater than the volume of the hydrogel capsules (VHC) in the container.
41. The sealed container of embodiment 39, wherein the ratio of VS to VHC is selected from the group consisting of:
   (i) between about 1.5 and about 100,
   (ii) between about 2 and about 75,
   (iii) between about 3 and about 50,
   (iv) between about 4 and about 40,
   (v) between about 5 and about 30, and
   (vi) between about 10 and about 20.
42. The sealed container of any one of embodiments 39 to 41, which is configured to be stored in a manner to allow substantially all of the hydrogel capsules in the composition to be distributed substantially uniformly across the bottom interior surface of the stored container in a capsule layer with a depth equal to about 1.00 to about 1.25 times the mean diameter of the capsules in the composition.
43. The sealed container of any one of embodiments 39 to 42, wherein all interior surfaces of the container consist essentially of fluorinated ethylene propylene (FEP) or polyethylene terephthalate glycol (PETG).
44. The sealed container of any one of embodiments 39 to 43, which is a flexible, rectangular bag which comprises a first port configured to allow addition of the composition to the bag and a second port configured to allow removal of a desired volume of the composition from the bag.
45. The sealed container of embodiment 44, wherein the mean capsule diameter of the hydrogel capsules in the container is about 1500 μm, the interior surface area is about 500 cm², and the total volume of the composition in the container is about 200 mL to about 500 mL.
46. The sealed container of any one of embodiments 39 to 45, wherein the mean diameter of the hydrogel capsules in the container is about 1500 μm and the ratio of VHC to VS is at least about 1:1, 1:2, 1:3 and less than about 1:40, e.g., about any of 1:5, 1:10, 1:15, 1:20, 1:25, 1:30, and 1:35.
47. A method of treating a subject in need of a therapeutic substance comprising providing a composition of any one of embodiments 33 to 38 and administering a therapeutically effective amount of the composition to the subject.
48. The method of embodiment 47, wherein the administering comprises disposing the effective amount into the intraperitoneal cavity of the subject.
49. The method of embodiments 47 or 48, wherein the subject is a human.
50. A method of making a sealed container comprising a hydrogel capsule composition, wherein the method comprises:
   (i) providing a population of hydrogel capsules encapsulating live mammalian cells, optionally wherein the mammalian cells are genetically modified to express and secrete a therapeutic substance, e.g., a therapeutic polypeptide;
   (ii) combining the population of hydrogel capsules with a pharmaceutically acceptable aqueous solution;
   (iii) placing a desired volume of the capsule composition into a biocompatible, sealable container in a manner that produces a capsule layer in which substantially all of the capsules in the composition volume are distributed substantially uniformly across the bottom of the container at a depth equal to about 1.00 to about 1.25 times the mean diameter of the capsules in the composition; and
   (iv) sealing the container.
51. The method of embodiment 50, wherein the aqueous solution has a pH of between 6.0 and 9.0 at 12° C. to 30° C. (e.g., about 15-25° C.), and comprises a calcium salt at an elemental calcium concentration of between about 1.0 millimolar (mM) and about 10 mM.
52. The method of embodiments 50 or 51, which prior to the sealing step comprises adding to the container a desired volume of the pharmaceutically acceptable aqueous solution to form a solution layer on top of the capsule layer.
53. The method of any one of embodiments 50 to 52, which further comprises storing the sealed container for a desired time period at a temperature of 2° C. to 30° C. or about 12° C. to 30° C. (e.g., about 15-25° C.). for a desired time period and assessing the viability of the encapsulated cells in the composition at one or more time points during the desired time period.

### EXAMPLES

In order that the disclosure described herein may be more fully understood, the following examples are set forth. The examples described in this application are offered to illustrate the genetically modified cells, compositions and implantable devices and methods provided herein, and are not to be construed in any way as limiting their scope.

### Example 1: Culturing of Exemplary Genetically-Modified ARPE-19 Cells for Encapsulation

Genetically modified ARPE-19 cells expressing one or more therapeutic substances described herein may be cultured to produce a composition of cells suitable for encapsulation in two compartment hydrogel capsules. The genetically-modified cells are grown in complete growth medium (DMEM:F12 with 10% FBS) in 150 cm² cell culture flasks or CellSTACK® Culture Chambers (Corning Inc., Corning, NY). To passage cells, the medium in the culture flask is aspirated, and the cell layer is briefly rinsed with phosphate buffered saline (pH 7.4, 137 mM NaCl, 2.7 mM KCl, 8 mM Na₂HPO₄, and 2 mM KH₂PO₄, Gibco). 5-10 mL of 0.05% (w/v) trypsin/0.53 mM EDTA solution (“TrypsinEDTA”) is added to the flask, and the cells are observed under an inverted microscope until the cell layer is dispersed, usually between 3-5 minutes. To avoid clumping, cells are handled with care and hitting or shaking the flask during the dispersion period is minimized. If the cells do not detach, the flasks are placed at 37° C. to facilitate dispersal. Once the cells disperse, 10 mL complete growth medium is added and the cells are aspirated by gentle pipetting. The cell suspension is transferred to a centrifuge tube and spun down at approximately 125×g for 5-10 minutes to remove TrypsinEDTA. The supernatant is discarded, and the cells are resuspended in fresh growth medium. Appropriate aliquots of cell suspension are added to new culture vessels, which are incubated at 37° C. The medium is renewed weekly.

### Example 2: Preparation of Exemplary Modified Polymers

Chemically-modified Polymer. A polymeric material may be chemically modified with a compound of Formula (I) (or pharmaceutically acceptable salt thereof) prior to formation of a device described herein (e.g., a hydrogel capsule). For example, in the case of alginate, the alginate carboxylic acid is activated for coupling to one or more amine-functionalized compounds to achieve an alginate modified with an afibrotic compound, e.g., a compound of Formula (I). The alginate polymer is dissolved in water (30 mL/gram polymer) and treated with 2-chloro-4,6-dimethoxy-1,3,5-triazine (0.5 eq) and N-methylmorpholine (1 eq). To this mixture is added a solution of the compound of interest (e.g., Compound 101 shown in Table 4) in acetonitrile (0.3M).

The amounts of the compound and coupling reagent added depends on the desired concentration of the compound bound to the alginate, e.g., conjugation density. A medium conjugation density of Compound 101 typically ranges from 2% to 5% N, while a high conjugation density of Compound 101 typically ranges from 5.1% to 8% N. To prepare a solution of low molecular weight alginate, chemically modified with a medium conjugation density of Compound 101 (CM-LMW-Alg-101-Medium polymer), the dissolved unmodified low molecular weight alginate (approximate MW<75 kDa, G:M ratio≥1.5) is treated with 2-chloro-4,6-dimethoxy-1,3,5-triazine (5.1 mmol/g alginate) and N-methylmorpholine (10.2 mmol/g alginate) and Compound 101 (5.4 mmol/g alginate). To prepare a solution of low molecular weight alginate, chemically modified with a high conjugation density of Compound 101 (CM-LMW-Alg-101-High polymer), the dissolved unmodified low-molecular weight alginate (approximate MW<75 kDa, G:M ratio≥1.5) is treated with 2-chloro-4,6-dimethoxy-1,3,5-triazine (5.1 mmol/g alginate) and N-methylmorpholine (10.2 mmol/g alginate) and Compound 101 (10.5 mmol/g alginate).

The reaction is warmed to 55° C. for 16 h, then cooled to room temperature and gently concentrated via rotary evaporation, then the residue is dissolved in water. The mixture is filtered through a bed of cyano-modified silica gel (Silicycle) and the filter cake is washed with water. The resulting solution is then extensively dialyzed (10,000 MWCO membrane) and the alginate solution is concentrated via lyophilization to provide the desired chemically-modified alginate as a solid or is concentrated using any technique suitable to produce a chemically modified alginate solution with a viscosity of 25 cP to 35 cP.

The conjugation density of a chemically modified alginate is measured by combustion analysis for percent nitrogen. The sample is prepared by dialyzing a solution of the chemically modified alginate against water (10,000 MWCO membrane) for 24 hours, replacing the water twice followed by lyophilization to a constant weight.

CBP-Alginates. A polymeric material may be covalently modified with a cell-binding peptide prior to formation of a device described herein (e.g., a hydrogel capsule described herein) using methods known in the art, see, e.g., Jeon O, et al., Tissue Eng Part A. 16:2915-2925 (2010) and Rowley, J. A. et al., Biomaterials 20:45-53 (1999).

For example, in the case of alginate, an alginate solution (1%, w/v) is prepared with 50 mM of 2-(N-morpholino)-ethanesulfonic acid hydrate buffer solution containing 0.5M NaCl at pH 6.5, and sequentially mixed with N-hydroxysuccinimide and 1-ethyl-3-[3-(dimethylamino)propyl]carbodiimide (EDC). The molar ratio of N-hydroxysuccinimide to EDC is 0.5:1.0. The peptide of interest is added to the alginate solution. The amounts of peptide and coupling reagent added depends on the desired concentration of the peptide bound to the alginate, e.g., peptide conjugation density. By increasing the amount of peptide and coupling reagent, higher conjugation density can be obtained. After reacting for 24 h, the reaction is purified by dialysis against ultrapure deionized water (diH2O) (MWCO 3500) for 3 days, treated with activated charcoal for 30 min, filtered (0.22 mm filter), and concentrated to the desired viscosity.

The conjugation density of a peptide-modified alginate is measured by combustion analysis for percent nitrogen. The sample is prepared by dialyzing a solution of the chemically modified alginate against water (10,000 MWCO membrane) for 24 hours, replacing the water twice followed by lyophilization to a constant weight.

### Example 3: Preparation of Exemplary Alginate Solutions for Making Hydrogel Capsules

70:30 mixture of chemically-modified and unmodified alginate. A low molecular weight alginate (PRONOVA™ VLVG alginate, NovaMatrix, Sandvika, Norway, cat. #4200506, approximate molecular weight<75 kDa; G:M ratio≥1.5) is chemically modified with Compound 101 to produce chemically modified low molecular weight alginate (CM-LMW-Alg-101) solution with a viscosity of 25 cp to 35 cP and a conjugation density of 5.1% to 8% N, as determined by combustion analysis for percent nitrogen. A solution of high molecular weight unmodified alginate (U-HMW-Alg) is prepared by dissolving unmodified alginate (PRONOVA™ SLG100, NovaMatrix, Sandvika, Norway, cat. #4202106, approximate molecular weight of 150 kDa-250 kDa) at 3% weight to volume in 0.9% saline. The CM-LMW-Alg solution is blended with the U-HMW-Alg solution at a volume ratio of 70% CM-LMW-Alg to 30% U-HMW-Alg (referred to herein as a 70:30 CM-Alg:UM-Alg solution).

Unmodified alginate solution. An unmodified medium molecular weight alginate (SLG20, NovaMatrix, Sandvika, Norway, cat. #4202006, approximate molecular weight of 75-150 kDa), is dissolved at 1.4% weight to volume in 0.9% saline to prepare a U-MMW-Alg solution.

Unmodified alginate solution. An unmodified medium molecular weight alginate (SLG20, NovaMatrix, Sandvika, Norway, cat. #4202006, approximate molecular weight of 75-150 kDa), is dissolved at 1.4% weight to volume in 0.9% saline to prepare a U-MMW-Alg solution.

Alginate Solution Comprising Cell Binding Sites. A solution of SLG20 alginate is modified with a peptide consisting of GRGDSP as described above and concentrated to a viscosity of about 100 cP. The amount of the peptide and coupling reagent used are selected to achieve a target peptide conjugation density of about 0.2 to 0.3, as measured by combustion analysis.

### Example 4: Formation of Exemplary Two-Compartment Hydrogel Capsules

Genetically modified cells may be encapsulated in two-compartment hydrogel capsules according to processes known in the art, e.g., as described in WO2021/113751. An exemplary protocol is described below.

Prior to fabricating hydrogel capsules, buffers and alginate solutions are sterilized by filtration through a 0.2-μm filter using aseptic processes.

Immediately before encapsulation, a desired volume of a composition comprising the cells (e.g., from a culture of the cells as described in Example 1) is centrifuged at 1,400 r.p.m. for 1 min and washed with calcium-free Krebs-Henseleit (KH) Buffer (4.7 mM KCl, 25 mM HEPES, 1.2 mM KH₂PO₄, 1.2 mM MgSO₄×7H₂O, 135 mM NaCl, pH≈7.4, ≈290 mOsm). After washing, the cells ae centrifuged again and all of the supernatant is aspirated. The cell pellet is resuspended in the GRGDSP-modified alginate solution described in Example 3 at a desired cell density (e.g., about 50 to 150 million suspended single cells per ml alginate solution).

To prepare two-compartment hydrogel millicapsules of about 1.5 mm diameter an electrostatic droplet generator is set up as follows: an ES series 0-100-kV, 20-watt high-voltage power generator (EQ series, Matsusada, NC, USA) is connected to the top and bottom of a coaxial needle. For capsules without an immunosuppressant, a suitable needle has an inner lumen of 22G, outer lumen of 18G, Rame-Hart Instrument Co., Succasunna, NJ, USA. To prepare capsules that co-encapsulate immunosuppressant particles in the inner compartment, the inner lumen of the coaxial needle may need to have a larger diameter to avoid needle clogging by the immunosuppressant particles, e.g., a useful coaxial needle has an inner lumen of 21G and an outer lumen of 17G, Ramé-Hart Instrument Co., Succasunna, NJ, USA).

The inner lumen is attached to a first 5-ml Luer-lock syringe (BD, NJ, USA), which is connected to a syringe pump (Pump 11 Pico Plus, Harvard Apparatus, Holliston, MA, USA) that is oriented vertically. The outer lumen is connected via a luer coupling to a second 5-ml Luer-lock syringe which is connected to a second syringe pump (Pump 11 Pico Plus) that is oriented horizontally. A first alginate solution containing the genetically modified cells (as single cells) suspended in a GRGDSP-modified alginate solution is placed in the first syringe and a cell-free alginate solution comprising a mixture of a chemically-modified alginate and unmodified alginate (e.g., 70:30 mixture described in Example 3) is placed in the second syringe. The two syringe pumps move the first and second alginate solutions from the syringes through both lumens of the coaxial needle and single droplets containing both alginate solutions are extruded from the needle into a glass dish containing a cross-linking solution. The settings of each Pico Plus syringe pump are 12.06 mm diameter and the flow rates of each pump are adjusted to achieve a flow rate ratio of 1:1 for the two alginate solutions. Thus, with the total flow rate set at 1 ml/h, the flow rate for each alginate solution was about 5 mL/h. Control (empty) capsules are prepared in the same manner except that the alginate solution used for the inner compartment is a cell-free solution.

After extrusion of the desired volumes of alginate solutions, the alginate droplets are crosslinked for five minutes in a cross-linking solution which contained 25 mM HTEPES buffer, 20 mM BaCl₂, 0.2M mannitol and 0.01 of poloxamer 188. Capsules that fall to the bottom of the crosslinking vessel are collected by pipetting into a conical tube. After the capsules settle in the tube, the crosslinking buffer is removed, and capsules are washed multiple times in an aqueous solution and stored at a desired temperature, e.g., room temperature.

### Example 5: Composition of Aqueous Storage Solutions Used in Examples 6 to 10

TABLE A
Inorganic salts and Carbon Sources

| Component | Solution A | Solution B | Solution C | Solution D | Solution E* |
|---|---|---|---|---|---|
| CaCl₂ | None | 0.30 g/L | 0.20 g/L | 0.20 g/L | 0.20 g/L |
| | | (Dihydrate) | (Dihydrate) | (Dihydrate) | (Anhyd) |
| NaCl | 5.25 g/L | 5.25 g/L | 6.00 g/L | 6.00 g/L | 6.40 g/L |
| NaOAc | 2.29 g/L | 2.29 g/L | None | None | None |
| KCl | 0.37 g/L | 0.37 g/L | 0.30 g/L | 0.30 g/L | 0.40 g/L |
| MgCl₂(H₂O)₆ | 0.30 g/L | 0.30 g/L | None | None | None |
| Fe(NO₃)₃(H₂O)₉ | None | None | None | None | 0.001 g/L |
| MgSO₄ (anhyd.) | None | None | None | None | 0.10 g/L |
| KCl | None | None | None | None | 0.40 g/L |
| NaHCO₃ | None | None | None | None | 3.70 g/L |
| NaH₂PO₄—H₂O | None | None | None | None | 0.13 g/L |
| Sodium | 5.18 g/L | 5.18 g/L | None | None | None |
| Gluconate | | | | | |
| Sodium | None | None | 0.31 g/L | 0.31 g/L | None |
| Lactate | | | | | |
| D-glucose | None | None | None | 0.05 g/L | 4.50 g/L |
| Sterile water | ✓ | ✓ | ✓ | ✓ | ✓ |
| Osmolality/ | 255-340 Osm/kg | 255-340 Osm/kg | 273 mOsm/L | 525 mOsm/L | 320-350 |
| Osmolarity | | | (calc) | (calc) | mOsm/kg |
| pH | 6.5-7.6 | 6.5-7.6 | 6.5 (6.0-7.5) | 5.0 (4.0-6.5) | 7.0-9.0 |

*Solution E includes the additional components listed in Table B below.

TABLE B
Amino Acids and Vitamins in Solution E

| | Concentration |
|---|---|
| Component | (g/L) |
| Glycine | 0.030 |
| L-Arginine HCl | 0.084 |
| L-Cystine 2HCl | 0.063 |
| L-Histidine HCl—H₂O | 0.042 |
| L-Isoleucine | 0.105 |
| L-Leucine | 0.105 |
| L-Lysine HCl | 0.146 |
| L-Methionine | 0.030 |
| L-Phenylalanine | 0.660 |
| L-Serine | 0.042 |
| L-Threonine | 0.095 |
| L-Tryptophan | 0.016 |
| L-Tyrosine Na₂(H₂O)₂ | 0.104 |
| L-Valine | 0.094 |
| Choline chloride | 0.004 |
| Ca D-pantothenate | 0.004 |
| Folic acid | 0.004 |
| Niacinamide | 0.004 |
| Pyridoxine HCl | 0.004 |
| Riboflavin | 0.0004 |
| Thiamine HCl | 0.004 |
| i-Inositol | 0.007 |

### Example 6: Viability of Encapsulated Cells is Improved by Presence of Calcium in Capsule Storage Solution

Two-compartment hydrogel capsules encapsulating ARPE-19 cells genetically modified to secrete a FVIII protein (“FVIII-spheres”) were prepared substantially as described in Example 4. Five milliliters (mL) of spheres were suspended in 63 mL of Solution A or Solution B and the resulting suspensions were placed into a 125 mL square, sterile Nalgene™ PETG bottle (ThermoFisher Scientific Cat. No. 342020-0125) laid horizontally and the spheres were allowed to settle as a 1.25× layer across the bottle bottom with the remaining storage solution forming a solution layer on top of the sphere layer. The sphere-containing bottles were stored in horizontal position at room temperature (e.g., about 25° C.) for three days and the viability of cells was assessed at several time points. The viability of cells was assessed by removing an aliquot of the sphere composition from each bottle, combining the aliquot with an alginate lyase solution to digest the alginate hydrogel and release the cells from the spheres, incubating the released cells with trypan blue stain, and counting the number of dead cells (i.e., stained with trypan blue) and live cells using an automated cell counter.

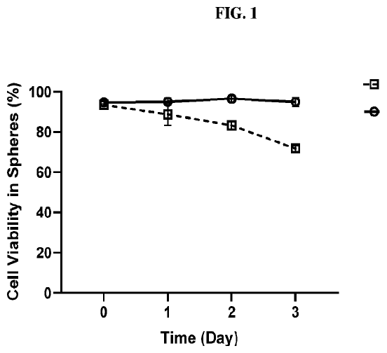

As shown in FIG. 1, cell viability in spheres stored in Solution A continually decreased over the 3 days of storage, while cell viability in capsules stored in Solution B remained essentially constant during the 3 storage days. Since the only difference between these two solutions is the presence of 2 mM calcium chloride in Solution B, the inventors herein hypothesized that calcium is important to maintain the viability of ARPE-19 cells encapsulated in hydrogel capsules.

Based on the results of additional experiments in which FVIII-spheres were stored in aqueous solutions containing varying amounts of calcium chloride (data not shown), the inventors herein believe that increased in vitro viability of encapsulated ARPE-19 cells can be achieved when spheres containing the cells are stored in an aqueous solution containing an elemental calcium concentration of between at least about 1.0 millimolar (mM) and about 10 mM compared to storage of the same spheres in the same solution that lacks calcium.

### Example 7: Osmolality and pH of Storage Solution Affects Productivity of Encapsulated Cells

FVIII-spheres were prepared substantially as described in Example 4. A suspension of 1 milliliter (mL) of spheres in 30 mL of Solution A containing 2-5 mM calcium (Ca-Sol A)) was placed into 30 mL square, sterile Nalgene™ PETG bottles (ThermoFisher Scientific, Cat. No. 342020-030) laid horizontally and a suspension of 5 milliliters (mL) of spheres in 63 mL of an aqueous storage solution (Solution C or Solution D) was placed into 125 mL square, sterile Nalgene™ PETG bottles (ThermoFisher Scientific Cat. No. 342020-0125) laid horizontally. The spheres in both size bottles were allowed to settle as a monolayer across the bottle bottom with the remaining storage solution forming a solution layer on top of the sphere layer. Following storage of the sphere-containing bottles in horizontal position at room temperature (e.g., about 25° C.) for four days, 0.25 mL of spheres were removed from each bottle and implanted into the intraperitoneal (IP) space of NSG™ mice (The Jackson Laboratory, Ellsworth, ME USA). Four mice were implanted per storage solution. As a control, 0.25 ml of FVIII-spheres suspended in Solution A containing 2-5 mM Calcium (Ca-Sol A) were implanted into the IP space of each of 4 NSG mice between 20-30 hours after sphere preparation. At 7 days post implant, the amount of plasma FVIII was determined for each cohort using an enzyme-linked immunoassay (ELISA) and the results are shown in FIG. 2.

Similar levels of FVIII were secreted from implanted spheres that had been stored in the Ca-Sol A or Solution C, while no plasma FVIII was detected in spheres stored in Solution D. Since the osmolality and pH of Solutions A and C are similar, and Solution D has a significantly higher osmolality and significantly lower pH, the inventors herein hypothesized that osmolality and pH of the aqueous storage solution are factors that can impact the productivity of cells encapsulated in hydrogel capsules.

### Example 8: Productivity of Encapsulated Cells can be Improved by Including Amino Acids and Vitamins in the Storage Solution

FVIII-spheres were prepared substantially as described in Example 4. A suspension of 20 milliliters (mL) of spheres in 400 mL of an aqueous storage solution (Solution B or Solution E) was placed into a rectangular PETG tray and the spheres were allowed to settle as a monolayer across the bottom of the tray and the remaining storage solution forming a solution layer on top of the sphere layer. Following storage of the sphere-containing trays at room temperature (e.g., about 25° C.) for up to 120 hours, 0.5 mL of spheres were removed from each tray and implanted into the intraperitoneal (IP) space of NSG mice. Three mice were implanted per storage solution and storage time, with spheres stored in Solution B implanted after 96 hours (t96) and spheres stored in Solution E implanted after 96 or 120 hours. As a control, 0.5 ml of FVIII-spheres suspended in the same storage solution (Solution E or Solution B) were implanted into the IP space of each of 3 NSG mice within 4 hours after sphere preparation. At 6 days post implant, the amount of plasma FVIII was determined for each cohort using an enzyme-linked immunoassay (ELISA) and the results are shown in FIG. 3.

### Example 9: Depth of Sphere Layer in Storage Container can Impact Productivity of Encapsulated Cells

FVIII-spheres were prepared substantially as described in Example 4. Three sphere suspensions containing different volumes of spheres in the same volume of Ca-Sol A were placed into rectangular PETG trays. The sphere volumes in each suspension were selected to provide sphere:solution ratios of 1:20, 1:10 and 1:5 and sphere settling across the bottom of the tray as a monolayer, bilayer or trilayer, respectively. The trays were stored for seven days at room temperature (e.g., about 25° C.). At the end of 1, 5 and 7 days, five spheres were removed from each tray and incubated in 100 microliters of culture media at 37° C. for 4 to 20 hours, and then the amount of FVIII secreted into the culture media determined by ELISA. The results are shown in FIG. 4, with each bar representing the average of duplicate samples for each tray configuration and storage time.

The results shown in FIG. 4 indicate that the thickness of the sphere layer on the bottom of the storage container can impact in vitro productivity of the encapsulated cells during storage, with the productivity maintained for spheres stored as a monolayer for seven days, while productivity decreased for spheres stored as a bi- or tri-layer.

### Example 10: Ratio of Sphere to Storage Solution in Storage Container has Minimal Impact on Productivity of Encapsulated Cells

FVIII-spheres were prepared substantially as described in Example 4. Four sphere suspensions containing different volumes of spheres and volumes of Ca-Sol A were placed into 125 mL square, sterile Nalgene™ PETG bottles (ThermoFisher Scientific Cat. No. 342020-0125). The sphere and storage solution volumes in each suspension were selected to produce four different storage configurations upon sphere settling: (1) sphere monolayer with 1:18 sphere:solution ratio; (2) sphere monolayer with 1:38 sphere:solution ratio; (3) sphere 1.25× layer with 1:6 sphere:solution ratio and (4) sphere 1.25× layer with 1:30 sphere:solution ratio. The bottles were then stored at room temperature (e.g., about 25° C.) for up to 4 days.

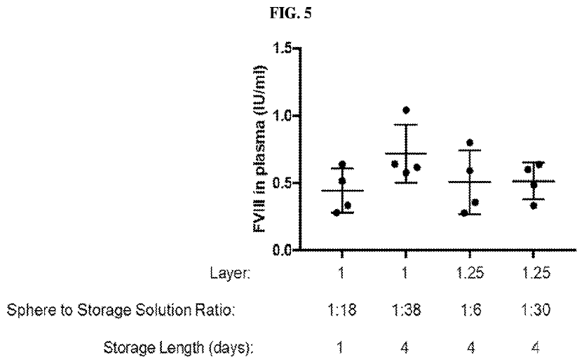

Following a desired storage period (1 day for storage configuration 1; 4 days for storage configurations 2 to 4), 0.5 mL spheres were removed from the bottles and implanted into the IP space of NSG mice, with four mice implanted per storage configuration. At 7 days post implant, the amount of plasma FVIII was determined for each mice cohort using an enzyme-linked immunoassay (ELISA) and the results are shown in FIG. 5. The results show that substantially similar amounts of FVIII were secreted from implanted spheres that had been stored as a monolayer or 1.25 layer in different amounts of storage solution.

### EQUIVALENTS AND SCOPE

This application refers to various issued patents, published patent applications, journal articles, and other publications, all of which are incorporated herein by reference in their entirety. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present disclosure, as defined in the following claims.

1. A composition comprising a population of hydrogel capsules disposed in a pharmaceutically acceptable aqueous solution, wherein each hydrogel capsule in the population encapsulates a plurality of live mammalian cells, wherein the solution has a pH of between 6.0 and 9.0 at 12° C. to 30° C. (e.g., about 15-25° C.), and comprises a calcium salt at an elemental calcium concentration of between about 1.0 millimolar (mM) and about 10 mM.

2. The composition of claim 1, wherein the solution has an osmolality of about 250 milliosmole/kg solution to about 350 milliosmole/kg solution.

3. The composition of claim 1, wherein each hydrogel capsule in the population comprises an ionically cross-linked alginate.

4. The composition of claim 1, wherein the pH of the solution is between 6.5 and 9.0 at 15° C. to 25° C.

5. The composition of claim 1, wherein the elemental calcium concentration is between an x value and a y value, wherein the x and y values are selected from the group consisting of:
(vii) x=about 1.1 mM and y=about 8.0 mM, 6.0 mM, 4.0 mM, or 2.0 mM;
(viii) x=about 1.2 mM and y=about 5 mM, 4 mM, 3 mM, or 2.0 mM;
(ix) x=about 1.2 mM and y=about 2.0 mM or 1.5 mM;
(x) x=about 1.3 mM and y=about 1.4 mM;
(xi) x=about 1.4 mM and y=about 4.0. mM, 3.0 mM, or 2.0 mM; and
(xii) x=about 1.5 mM and y=about 2.5 mM.

6. The composition of claim 1, wherein the solution further comprises at least one carbon source (e.g., a sugar (e.g., dextrose, glucose, galactose, hexose, fructose, maltose), glycerol, glutamine, pyruvate or salt thereof).

7. The composition of claim 1, wherein the solution further comprises a buffering agent which comprises one or more of an acetate salt (e.g., sodium acetate), a gluconate salt (e.g., sodium gluconate), a phosphate salt (e.g., sodium phosphate monobasic), a bicarbonate salt (e.g., sodium bicarbonate), and a lactate salt (e.g., sodium lactate).

8. The composition of claim 7, wherein the buffering agent comprises sodium acetate and sodium gluconate.

9. The composition of claim 8, wherein the buffering agent consists essentially of about 0.5-5 g/L sodium acetate (e.g., 2 g/L, e.g., 2.29 g/L sodium acetate) and about 0.5-10 g/L sodium gluconate (e.g., 5 g/L, e.g., 5.18 g/L) sodium gluconate.

10. The composition of claim 6, wherein the carbon source is glucose, and the solution does not contain any added glutamine or phenol red.

11. The composition of claim 7, wherein the buffering agent comprises sodium bicarbonate and sodium phosphate and the solution does not contain any added HEPES or sodium pyruvate.

12. The composition of claim 1, wherein the calcium salt is calcium chloride.

13. The composition of claim 1, wherein the solution comprises about 1.5 mM to 2.5 mM calcium chloride, about 5 mM to about 25 mM D-glucose, and about 40 mM to about 50 mM sodium bicarbonate.

14. The composition of claim 1, wherein the solution further comprises:
(vi) a magnesium compound (e.g., magnesium chloride or magnesium sulfate):
(vii) a potassium compound (e.g., potassium chloride),
(viii) sodium chloride; and
(ix) a set of amino acids, which comprises histidine, isoleucine, leucine, lysine, methionine, phenylalanine, threonine, tryptophan, and valine; and
(x) a set of vitamins, which comprises a vitamin B1 compound (e.g., thiamine or a thiamine salt, e.g., thiamine hydrochloride), a vitamin B3 compound (e.g., nicotinic acid or niacinamide) and a vitamin B6 compound (e.g., pyroxidine or a pyroxidine salt, e.g., pyroxidine hydrochloride).

15. The composition of claim 14, wherein the set of amino acids also comprises arginine, glycine, cystine, serine, and tyrosine and the set of vitamins also comprises choline or a choline salt (e.g., choline chloride), a vitamin B5 compound (e.g., pantotheneic acid or calcium pantothenate), a folate compound (e.g., folic acid), riboflavin and i-inositol.

16. The composition of claim 1, wherein each hydrogel capsule in the population has a sphere-like or spherical shape and comprises:
(c) a cell-containing compartment which comprises the plurality of live cells encapsulated in a first polymer composition; and
(d) a barrier compartment surrounding the cell-containing compartment and comprising a second polymer composition which comprises the ionically cross-linked alginate;
wherein the mean diameter of the hydrogel capsules in the population is about 500 micrometers (μm) to about 5000 μm, about 1000 (μm) to about 3000 μm, about 1100 μm to about 2500 μm, about 1200 μm to about 2300 μm, about 1300 μm to about 2100 μm, about 1400 μm to about 2000 μm, about 1400 μm to about 1900 μm, about 1400 μm to about 1800 μm.

17. The composition of claim 16, wherein the mean capsule diameter of the hydrogel capsules in the population is 1400 to 2000 μm.

18. The composition of claim 16, wherein the average thickness of the barrier compartment is about 10 to about 300 microns, about 20 to about 150 microns, or about 40 to about 75 microns.

19. The composition of claim 16, wherein the first polymer composition comprises an alginate covalently modified with a cell-contacting peptide via a linker and wherein the cross-linked alginate in the barrier compartment comprises an alginate covalently modified with at least one afibrotic compound, optionally a compound shown in Table 1.

20. The composition of claim 19, wherein the ionically cross-linked alginate in the barrier compartment comprises barium ions as at least one cross-linking agent.

21. The composition of claim 19, wherein the ionically cross-linked alginate in the barrier compartment comprises a mixture of the covalently modified alginate and an unmodified alginate.

22. The composition of claim 20, wherein the covalently modified alginate in the cell-containing compartment is ionically cross-linked with barium ions as at least one cross-linking agent.

23. The composition of claim 21, wherein:
(g) the mean diameter of the hydrogel capsules in the population is 1400 μm to 2000 μm, or 1400 μm to 1600 μm, or 1000 μm to 1200 μm;
(h) the alginate in the first polymer composition has a molecular weight of 150 to 250 kDa and a G:M ratio of greater than or equal to 1.5;
(i) the cell-contacting peptide consists of RGDSP and the linker is a single glycine residue attached to the N-terminus of the cell-contacting peptide;
(j) the alginate in the covalently-modified alginate in the barrier compartment has a molecular weight of <75 kDa and a G:M ratio of greater than or equal to 1.5;
(k) the afibrotic compound is 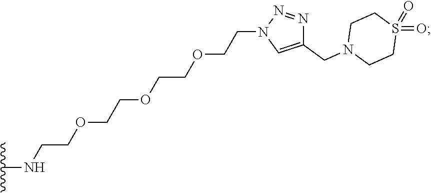 [SMILES: *NCCOCCOCCOCCn1cc(CN2CCS(=O)(=O)CC2)nn1]
and
(l) the unmodified alginate in the barrier compartment has a molecular weight of 150 kDa to 250 kDa and a G:M ratio of greater than or equal to 1.5.

24. The composition of claim 1, wherein the live mammalian cells are human cells.

25. The composition of claim 1, wherein the cells are derived from an induced pluripotent stem cell.

26. The composition of claim 1, wherein the cells are derived from an RPE cell, optionally derived from an ARPE-19 cell.

27. The composition of claim 1, wherein the encapsulated cells comprise single cells.

28. The composition of claim 1, wherein the encapsulated cells comprise one or more cell clusters.

29. The composition of claim 1, wherein the encapsulated cells comprise cells disposed on a microbead.

30. The composition of claim 1, wherein the live mammalian cells are genetically modified to express and secrete a therapeutic substance, e.g., a therapeutic polypeptide.

31. The composition of claim 1, wherein the mammalian cells comprise an exogenous nucleotide sequence which encodes a therapeutic polypeptide, optionally wherein the therapeutic polypeptide is a growth factor, a blood coagulation factor, an enzyme, a cytokine, a cytokine receptor, an antibody or antigen-binding fragment thereof.

32. The composition of claim 31, wherein the therapeutic polypeptide is an FVIII protein (e.g., an FVIII BDD protein), a FIX protein, or a FVII protein.

33. The composition of claim 31, wherein the therapeutic polypeptide is a GLA protein, an IDUA protein, an IDS protein, an ARSB protein, or a GBA protein.

34. The composition of claim 17, wherein the plurality of live mammalian cells is between about 5,000 to about 250,000 cells, about 10,000 to about 125,000 cells, about 20,000 to about 75,000 cells, about 12,500 to about 40,000 cells, or about 15,000 to about 30,000 cells.

35. The composition of claim 1, which comprises about 200 to about 400 of the hydrogel capsules per milliliter of the pharmaceutically acceptable solution.

36. A sealed container comprising the composition of any one of the above claims.

37. The sealed container of claim 36, wherein the volume of the aqueous solution (VS) in the container is about equal to or greater than the volume of the hydrogel capsules (VHC) in the container.

38. The sealed container of claim 37, wherein the ratio of VS to VHC is selected from the group consisting of:
(vii) between about 1.5 and about 100,
(viii) between about 2 and about 75,
(ix) between about 3 and about 50,
(x) between about 4 and about 40,
(xi) between about 5 and about 30, and
(xii) between about 10 and about 20.

39. The sealed container of claim 37, which is configured to be stored in a manner to allow substantially all of the hydrogel capsules in the composition to be distributed substantially uniformly across the bottom interior surface of the stored container in a capsule layer with a depth equal to about 1.00 to about 1.25 times the mean diameter of the capsules in the composition.

40. The sealed container of claim 37, wherein all interior surfaces of the container consist essentially of fluorinated ethylene propylene (FEP) or polyethylene terephthalate glycol (PETG).

41. The sealed container of claim 37, which is a flexible, rectangular bag which comprises a first port configured to allow addition of the composition to the bag and a second port configured to allow removal of a desired volume of the composition from the bag.

42. The sealed container of claim 41, wherein the mean capsule diameter of the hydrogel capsules in the container is about 1500 μm, the interior surface area is about 500 cm², and the total volume of the composition in the container is about 200 mL to about 500 mL.

43. The sealed container of claim 37, wherein the mean diameter of the hydrogel capsules in the container is about 1500 μm and the ratio of VHC to VS is at least about 1:1, 1:2, 1:3 and less than about 1:40, e.g., about any of 1:5, 1:10, 1:15, 1:20, 1:25, 1:30, and 1:35.

44. A method of treating a subject in need of a therapeutic substance comprising providing a composition of claim 1 and administering a therapeutically effective amount of the composition to the subject.

45. The method of claim 44, wherein the administering comprises disposing the effective amount into the intraperitoneal cavity of the subject.

46. The method of claim 44, wherein the subject is a human.

47. A method of making a sealed container comprising a hydrogel capsule composition, wherein the method comprises:
(i) providing a population of hydrogel capsules encapsulating live mammalian cells, optionally wherein the mammalian cells are genetically modified to express and secrete a therapeutic substance, e.g., a therapeutic polypeptide;
(ii) combining the population of hydrogel capsules with a pharmaceutically acceptable aqueous solution;
(iii) placing a desired volume of the capsule composition into a biocompatible, sealable container in a manner that produces a capsule layer in which substantially all of the capsules in the composition volume are distributed substantially uniformly across the bottom of the container at a depth equal to about 1.00 to about 1.25 times the mean diameter of the capsules in the composition; and
(iv) sealing the container.

48. The method of claim 47, wherein the aqueous solution has a pH of between 6.0 and 9.0 at 12° C. to 30° C. (e.g., about 15-25° C.), and comprises a calcium salt at an elemental calcium concentration of between about 1.0 millimolar (mM) and about 10 mM.

49. The method of claim 47, which prior to the sealing step comprises adding to the container a desired volume of the pharmaceutically acceptable aqueous solution to form a solution layer on top of the capsule layer.

50. The method of claim 49, which further comprises storing the sealed container for a desired time period at a temperature of 2° C. to 30° C. or about 12° C. to 30° C. (e.g., about 15-25° C.). for a desired time period and assessing the viability of the encapsulated cells in the composition at one or more time points during the desired time period.
